(19) United States
(12) Patent Application Publication
(10) Pub. No.: US 2011/0077251 A1
(43) Pub. Date: Mar. 31, 2011

(54) NOVEL 17 BETA LUPANE DERIVATIVES

(76) Inventors: Christophe Moinet, Noyal Chatillon (FR); Bingcan Liu, St-Laurent (CA); Liliane Halab, Laval (CA); Nathalie Turcotte, Rosemere (CA); Real Denis, Laval (CA); Carl Poisson, Montreal (CA); Paul Nguyen-Ba, Laprairie (CA); Monica Bubenik, Mascouche (CA); Laval Chan Chun Kong, Kirkland (CA)

(21) Appl. No.: 12/856,848

(22) Filed: Aug. 16, 2010

(30) Foreign Application Priority Data
Feb. 13, 2009 (CA) PCT/CA09/00173

(51) Int. Cl. A61K 31/5375; C07C 271/24; C07C 69/34; C07D 295/195; C07C 69/74; C07D 211/60; C07D 211/32; C07D 271/113; C07D 277/46; C07D 309/06; C07D 213/75; C07D 207/04; C07D 231/14; A61K 31/215; A61K 31/495; A61K 31/4453; A61K 31/4245; A61K 31/426; A61K 31/351; A61K 31/4409; A61K 31/40; A61K 31/415; A61P 31/18

(52) U.S. Cl. 514/237.5; 560/6; 560/194; 544/154; 560/122; 560/127; 544/380; 546/195; 548/143; 548/196; 549/426; 546/285; 548/528; 548/374.1; 514/510; 514/255.01; 514/319; 514/364; 514/371; 514/459; 514/353; 514/423; 514/406

(57) ABSTRACT

The invention relates to 17β lupane derivatives of formula (I):

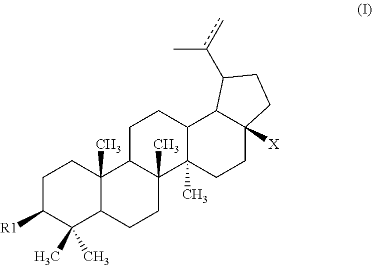

wherein R¹ and X are as defined herein, and pharmaceutically acceptable salts and solvates thereof. These compounds exhibit significant anti-HIV activity. Thus, the invention also relates to methods for prevention or treatment of HIV infection by administering therapeutically effective amounts of a compound of formula (I), or a pharmaceutically acceptable salt or solvate thereof to a subject in need of such treatment.

This application is related to application Ser. No. 61/028,533, filed Feb. 14, 2008, the entire disclosure of which is incorporated by reference.

Infection by the Human immunodeficiency virus (HIV) can lead to the Acquired ImmunoDeficiency Syndrome (AIDS), an incurable and life threatening condition which requires life-long treatment. It is estimated that the HIV/AIDS pandemic has resulted in the deaths of more than 25 million people since it was first recognized in 1981 and according to a UNAIDS report, an estimated 40 million people worldwide are infected with HIV and about 2.5 million lost their lives to AIDS in 2005. There is presently no effective vaccine for HIV. HIV primarily infects T cells, macrophages and other important components of the immune system resulting in the gradual loss of cell-mediated immunity and as result, HIV patients become increasingly more susceptible to numerous opportunistic infections and tumors and if left untreated, death usually results within 10 years following infection.

The viral life cycle initiates with attachment of HIV gp120 surface protein to the CD4 receptors present of the T-cells. This event triggers a conformational change which exposes an additional binding site on gp120 and results with an interaction with the chemokine co-receptors (CCR5 and CXCR4). Another conformational change arising from co-receptor binding results in fusion of the cellular and viral membranes and release of the virion into the cell. After uncoating and release of the viral genome in the cytoplasm, viral reverse transcriptase (RT) then converts RNA into double stranded DNA which is then integrated into the host genome by the action of HIV integrase. The proviral DNA is then transcribed and translated by host cellular system to express HIV RNA and HIV proteins which are then directed to the cell membrane where they assemble and bud as immature virions. During or soon after the budding process, the viral protease cleaves specific sites in Gag and Gag-Pol releasing essential viral proteins and enzymes such as capsid, nucleocapsid, reverse transcriptase, integrase and spacer peptides SP1 and SP2. This last step is crucial for generating functional viral enzymes and also for the formation of the mature conical HIV capsid.

A number of antiviral agents have been developed to interfere with various stages of viral replication. For example, viral entry can be blocked with T-20 or Maraviroc and post entry steps such as reverse transcription can be blocked with nucleoside RT inhibitors (examples: Lamivudine, Tenofovir, Zidovudine, Didanosine, Emtricitabine, Abacavir) or nonnucleoside RT inhibitors (examples: Nevirapine, Efavirenz and Delavirdine). Integration can be blocked by Raltegravir and HIV proteolytic activity can be inhibited by protease inhibitors such as Saquinavir, Indinavir, Amprenavir, Darunavir, Lopinavir, Atazanavir, and Nelfinavir. Other experimental agents such as Vicriviroc (CCR5), Elvitegravir (integrase), Etravirine (RT), Apricitabine (RT), Bevirimat (maturation) are presently under investigation. The use of combinations of antiretroviral agents have been particularly effective in halting replication to undetectable levels and have led to markedly improved health and life span of HIV/AIDS patients. Nevertheless the appearance of drug resistant viruses after long term therapy is a major concern and there is still a major need for additional drugs in order to provide additional options for these patients facing these issues.

Triterpenoid derivatives have been shown to possess anti-viral properties. For example, moronic acid (D. Yu, et al. J. Med. Chem. 2006, 49, 5462-5469), oleanolic acid (H. Assefa, et al. Bioorg. Med. Chem. Lett. 1999, 9, 1889-1894), platanic acid (T. Fujioka, et al. J. Nat. Prod. 1994, 57, 243-247), betulonic acid (O. B. Flekhter, et al. Russ. J. Bioorg. Chem. 2004, 30, 80-88) and betulinic acid (I.-C. Sun, et al. Bioorg. Med. Chem. Lett. 1998, 8, 1267-1272) derivatives were shown to have anti-HIV-1 activities. Certain C-17 modified betulin derivatives are known and some of them have been reported as exhibiting anti herpes simplex type 1 and anti-influenza activity (O. B. Flekhter, et al. Russ. J. Bioorg. Chem. 2003, 29, 655-661) and also anti HIV activity (I.-C. Sun, et al. J. Med. Chem. 1998, 41, 4648-4657 and Feng Li, et al. Virology, 2006, 356, 217-224).

This invention relates to 17β lupane derivatives and the discovery that these novel modified triterpenoid derivatives possess significant anti-HIV activity.

The present invention relates to a compound of formula (I):

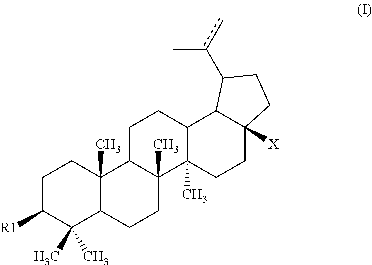

wherein;

R₁ is

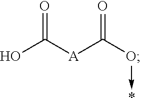

A is C₁₋₈ alkyl, C₂₋₈ alkenyl, or —(CH₂)₁₋₂O(CH₂)₁₋₂—;

X is

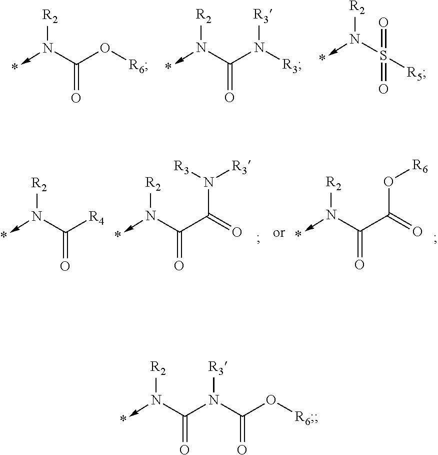

R₂ is H, C₁₋₁₂ alkyl which is unsubstituted or substituted one or more times by R₁₀, C₂₋₁₂ alkenyl which is unsubstituted or substituted one or more times by R₁₀, or C₂₋₁₂ alkynyl which is unsubstituted or substituted one or more times by R₁₀;

R₃ and R₃′ are each independently H, C₁₋₁₂ alkyl which is unsubstituted or substituted one or more times by R₁₀, C₂₋₁₂ alkenyl which is unsubstituted or substituted one or more times by R₁₀, C₂₋₁₂ alkynyl which is unsubstituted or substituted one or more times by R₁₀, C₆₋₁₄ aryl which is unsubstituted or substituted one or more times by R₁₁, C₇₋₁₆ aralkyl which is unsubstituted or substituted one or more times by R₁₁, 5-12 member heteroaryl which is unsubstituted or substituted one or more times by R₁₁, 6-18 member heteroaralkyl which is unsubstituted or substituted one or more times by R₁₁, 3-12 member heterocycle which is unsubstituted or substituted one or more times by R₁₂, or 4-18 member heterocycle-alkyl which is unsubstituted or substituted one or more times by R₁₂;

R₃ and R₃′ can also be taken together to form 5-12 member heteroaryl which is unsubstituted or substituted one or more times by R₁₁, or a 3-12 member heterocycle which is unsubstituted or substituted one or more times by R₁₂;

R₄ is C₁₋₁₂ alkyl which is unsubstituted or substituted one or more times by R₁₀, C₂₋₁₂ alkenyl which is unsubstituted or substituted one or more times by R₁₀, C₂₋₁₂ alkynyl which is unsubstituted or substituted one or more times by R₁₀, C₆₋₁₄ aryl which is unsubstituted or substituted one or more times by R₁₁, C₇₋₁₆ aralkyl which is unsubstituted or substituted one or more times by R₁₁, 5-12 member heteroaryl which is unsubstituted or substituted one or more times by R₁₁, 6-18 member heteroaralkyl which is unsubstituted or substituted one or more times by R₁₁, 3-12 member heterocycle which is unsubstituted or substituted one or more times by R₁₂, or 4-18 member heterocycle-alkyl which is unsubstituted or substituted one or more times by R₁₂;

R₅ and R₆ are each independently C₁₋₁₂ alkyl which is unsubstituted or substituted one or more times by R₁₀, C₂₋₁₂ alkenyl which is unsubstituted or substituted one or more times by R₁₀, C₂₋₁₂ alkynyl which is unsubstituted or substituted one or more times by R₁₀, C₆₋₁₄ aryl which is unsubstituted or substituted one or more times by R₁₁, C₇₋₁₆ aralkyl which is unsubstituted or substituted one or more times by R₁₁, 5-12 member heteroaryl which is unsubstituted or substituted one or more times by R₁₁, 6-18 member heteroaralkyl which is unsubstituted or substituted one or more times by R₁₁, 3-12 member heterocycle which is unsubstituted or substituted one or more times by R₁₂, or 4-18 member heterocycle-alkyl which is unsubstituted or substituted one or more times by R₁₂;
R¹⁰ is halogen, oxo, C₁₋₆ alkoxy, —NH₂, —NH(C₁₋₄ alkyl)₂, —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄ alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino;
R¹¹ is halogen, C₁₋₆ alkyl, halogenated C₁₋₆ alkyl, C₂₋₆ alkenyl, C₂₋₆ alkynyl, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino; and
R¹² is halogen, oxo, C₁₋₆ alkyl, halogenated C₁₋₆ alkyl, C₂₋₆ alkenyl, C₂₋₆ alkynyl, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino;

or a pharmaceutically acceptable salt thereof.

In a further embodiment, the compounds of the invention are represented by formula (Ia)

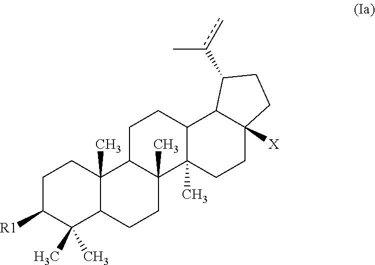

wherein R₁ and X are as defined herein.

In a further embodiment, the compounds of the invention are represented by formula (Ib) or (Ic)

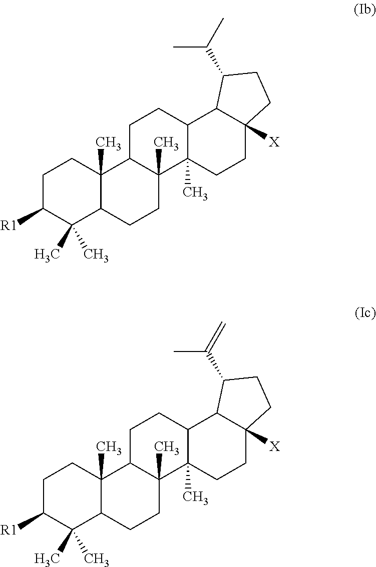

wherein R₁ and X are as defined herein.

In a further embodiment, the compounds of the invention are represented by formula (I), (Ia), (Ib) or (Ic)

wherein X is:

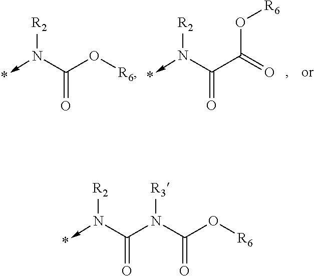

In a further embodiment, the compounds of the invention are represented by formula (I), (Ia), (Ib) or (Ic)

wherein X is:

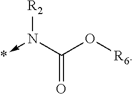

In a further embodiment, the compounds of the invention are represented by formula (I), (Ia), (Ib) or (Ic)

wherein X is:

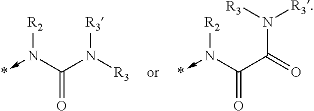

In a further embodiment, the compounds of the invention are represented by formula (I), (Ia), (Ib) or (Ic)

wherein X is:

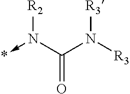

In a further embodiment, the compounds of the invention are represented by formula (I), (Ia), (Ib) or (Ic)

wherein X is:

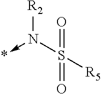

In a further embodiment, the compounds of the invention are represented by formula (I), (Ia), (Ib) or (Ic)

wherein X is:

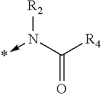

In a further embodiment, the compounds of the invention are represented by formula (I), (Ia), (Ib) or (Ic)

wherein X is:

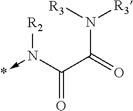

In a further embodiment, the compounds of the invention are represented by formula (I), (Ia), (Ib) or (Ic)

wherein X is:

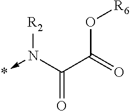

In a further embodiment, the compounds of the invention are represented by formula (I), (Ia), (Ib) or (Ic)

wherein X is:

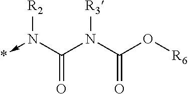

In a further embodiment, the compounds of the invention are represented by formula (I), (Ia), (Ib) or (Ic) wherein the following embodiments are present alone or in combination:

R₁ is

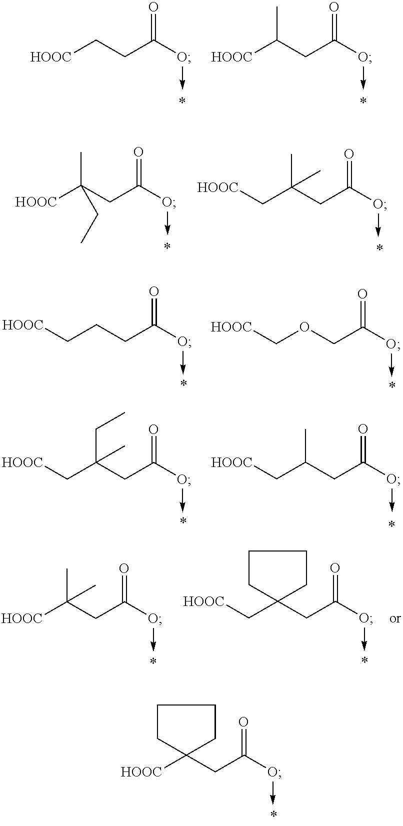

R₁ is O-succinyl, O-glutaryl, 0-3′-methylglutaryl, O-3′-methylsuccinyl, O-3′,3′-dimethylsuccinyl, O-3′,3′-dimethylglutaryl, O-2′,2′-dimethylmalonyl, O-2′,3′-dihydroxysuccinyl, O-2′,3′-dimethylsuccinyl, O-2′,2′,3′,3′-tetramethylsuccinyl, O-2′-methylsuccinyl, or O-2′,2′-dimethylsuccinyl.

R₁ is O-succinyl, O-glutaryl, O-3′-methylglutaryl, O-3′-methylsuccinyl, O-3′,3′-dimethylsuccinyl, O-3′,3′-dimethylglutaryl, O-2′,2′-dimethylmalonyl, O-2′,3′-dihydroxysuccinyl, O-2′,2′,3′,3′-tetramethylsuccinyl, or O-2′,2′-dimethylsuccinyl.

R₁ is O-3′,3′-dimethylsuccinyl.

R₂ is H or C₁₋₁₂ alkyl which is unsubstituted or substituted one or more times by R₁₀.

R₂ is H or C₁₋₆ alkyl which is unsubstituted or substituted one or more times by R₁₀.

R₂ is H, methyl, ethyl, propyl, isopropyl, butyl, sec-butyl, tert-butyl, cyclopropyl, cyclobutyl, cyclopentyl, or cyclohexyl.

R₂ is methyl, ethyl, propyl, isopropyl, butyl, sec-butyl, or tert-butyl.

R₂ is methyl.

R₂ is H.

R₃, R₄, R₅ and R₆ are each independently C₁₋₁₂ alkyl which is unsubstituted or substituted one or more times by R¹⁰, C₆ aryl which is unsubstituted or substituted one or more times by R¹¹, C₇₋₉ aralkyl which is unsubstituted or substituted one or more times by R¹¹, 5-6 member heteroaryl which is unsubstituted or substituted one or more times by R¹¹, 7-8 member heteroaralkyl which is unsubstituted or substituted one or more times by R¹¹, 5-6 member heterocycle which is unsubstituted or substituted one or more times by R¹², or 7-8 member heterocycle-alkyl which is unsubstituted or substituted one or more times by R¹².

R₃, R₄, R₅ and R₆ are each independently C₁₋₆ alkyl which is unsubstituted or substituted one or more times by R¹⁰, phenyl which is unsubstituted or substituted one or more times by R¹¹, benzyl which is unsubstituted or substituted one or more times by R¹¹, 5-6 member heteroaryl which is unsubstituted or substituted one or more times by R¹¹, 7-8 member heteroaralkyl which is unsubstituted or substituted one or more times by R¹¹, 5-6 member heterocycle which is unsubstituted or substituted one or more times by R¹², or 7-8 member heterocycle-alkyl which is unsubstituted or substituted one or more times by R¹².

R₃, R₄, R₅ and R₆ are each independently C₁₋₁₂ alkyl which is unsubstituted or substituted one or more times by R¹⁰.

R₃, R₄, R₅ and R₆ are each independently C₁₋₆ alkyl which is unsubstituted or substituted one or more times by R¹⁰.

R₃, R₄, R₅ and R₆ are each independently methyl, ethyl, propyl, isopropyl, butyl, sec-butyl, tert-butyl, cyclopropyl, cyclobutyl, cyclopentyl, or cyclohexyl.

R₃, R₄, R₅ and R₆ are each independently is methyl, ethyl, propyl, isopropyl, butyl, sec-butyl, or tert-butyl.

R₃, R₄, R₅ and R₆ are each independently phenyl which is unsubstituted or substituted one or more times by R₁₁.

R₃, R₄, R₅ and R₆ are each independently phenyl.

R₃, R₄, R₅ and R₆ are each independently benzyl which is unsubstituted or substituted one or more times by R₁₁.

R₃, R₄, R₅ and R₆ are each independently benzyl.

R₃, R₄, R₅ and R₆ are each independently 5-6 member heteroaryl which is unsubstituted or substituted one or more times by R¹¹.

R₃, R₄, R₅ and R₆ are each independently pyridyl which is unsubstituted or substituted one or more times by R¹¹.

R₃, R₄, R₅ and R₆ are each independently 7-8 member heteroaralkyl which is unsubstituted or substituted one or more times by R¹¹.

R₃, R₄, R₅ and R₆ are each independently —CH₂-pyridyl which is unsubstituted or substituted one or more times by R¹¹.

R₃, R₄, R₅ and R₆ are each independently —CH₂-cyclopropyl, —CH₂-cyclopentyl, —CH₂CH₂-cyclopentyl, —CH₂-cyclohexyl, —CH₂-pyridinyl, piperidynyl, —CH₂-piperidynyl, piperazinyl, thiophenyl, morpholino, oxadiazole, pyrimidinyl, pyranyl, pyrazinyl, thiazole, and pyrazole, which are unsubstituted or substituted by one or more substituents chosen from a halogen, C₁₋₄ alkyl, C₁₋₄ alkyloxy, CF₃, COC₁₋₄ alkyl, COOH, COOC₁₋₄ alkyl, cyano, NH₂, nitro, NH(C₁₋₆ alkyl), and N(C₁₋₆alkyl)₂.

R₃, R₄, R₅ and R₆ are each independently piperidynyl, piperazinyl, tetrahydropyranyl, and pyrrolidinyl which are unsubstituted or substituted one or more times by R₁₂.

R₃, R₄, R₅ and R₆ are each independently oxadiazolyl, thiazolyl, pyridinyl, oxadiazolyl and pyrazolyl, which are unsubstituted or substituted one or more times by R₁₁.

R₃ and R₃′ are each independently H, C₁₋₁₂ alkyl which is unsubstituted or substituted one or more times by R₁₀, C₆₋₁₄ aryl which is unsubstituted or substituted one or more times by R₁₁, C₇₋₁₆ aralkyl which is unsubstituted or substituted one or more times by R₁₁, or 5-12 member heteroaryl which is unsubstituted or substituted one or more times by R₁₁.

R₃ is C₁₋₁₂ alkyl which is unsubstituted or substituted one or more times by R¹⁰, C₂₋₁₂ alkenyl which is unsubstituted or substituted one or more times by R¹⁰, C₂₋₁₂ alkynyl which is unsubstituted or substituted one or more times by R¹⁰, C₆₋₁₄ aryl which is unsubstituted or substituted one or more times by R¹¹, C₇₋₁₆ aralkyl which is unsubstituted or substituted one or more times by R¹¹, 5-12 member heteroaryl which is unsubstituted or substituted one or more times by R¹¹, 6-18 member heteroaralkyl which is unsubstituted or substituted one or more times by R¹¹, 3-12 member heterocycle which is unsubstituted or substituted one or more times by R¹², or 4-18 member heterocycle-alkyl which is unsubstituted or substituted one or more times by R¹².

R₃ is C₁₋₁₂ alkyl which is unsubstituted or substituted one or more times by R¹⁰, C₆ aryl which is unsubstituted or substituted one or more times by R¹¹, C₇₋₉ aralkyl which is unsubstituted or substituted one or more times by R¹¹, 5-6 member heteroaryl which is unsubstituted or substituted one or more times by R¹¹, 7-8 member heteroaralkyl which is unsubstituted or substituted one or more times by R¹¹, 5-6 member heterocycle which is unsubstituted or substituted one or more times by R¹², or 7-8 member heterocycle-alkyl which is unsubstituted or substituted one or more times by R¹².

R₃ is C₁₋₆ alkyl which is unsubstituted or substituted one or more times by R¹⁰, phenyl which is unsubstituted or substituted one or more times by R¹¹, benzyl which is unsubstituted or substituted one or more times by R¹¹, 5-6 member heteroaryl which is unsubstituted or substituted one or more times by R¹¹, 7-8 member heteroaralkyl which is unsubstituted or substituted one or more times by R¹¹, 5-6 member heterocycle which is unsubstituted or substituted one or more times by R¹², or 7-8 member heterocycle-alkyl which is unsubstituted or substituted one or more times by R¹².

R₃ is C₁₋₁₂ alkyl which is unsubstituted or substituted one or more times by R₁₀, C₆₋₁₄ aryl which is unsubstituted or substituted one or more times by R₁₁, C₇₋₁₆ aralkyl which is unsubstituted or substituted one or more times by R₁₁, or 5-12 member heteroaryl which is unsubstituted or substituted one or more times by R₁₁.

R₃ is C₁₋₆ alkyl which is unsubstituted or substituted one or more times by R₁₀, phenyl which is unsubstituted or substituted one or more times by R₁₁, benzyl which is unsubstituted or substituted one or more times by R₁₁, or 6 member heteroaryl which is unsubstituted or substituted one or more times by R₁₁.

R₃ is C₁₋₆ alkyl which is unsubstituted or substituted one or more times by R₁₀, phenyl which is unsubstituted or substituted one or more times by R₁₁, benzyl which is unsubstituted or substituted one or more times by R₁₁, or pyridyl which is unsubstituted or substituted one or more times by R₁₁.

R₃ is C₁₋₆ alkyl which is unsubstituted or substituted one or more times by R₁₀ (e.g., —CH(isopropyl)COOH or —CH(isopropyl)COOCH₃).

R₃ is piperidynyl, piperazinyl, tetrahydropyranyl, and pyrrolidinyl which are unsubstituted or substituted one or more times by R₁₂.

R₃ is oxadiazolyl, thiazolyl, pyridinyl, oxadiazolyl and pyrazolyl, which are unsubstituted or substituted one or more times by R₁₁.

R₃ is 5-6 member heteroaryl which is unsubstituted or substituted one or more times by R¹¹.

R₃ is oxadiazole which is unsubstituted or substituted one or more times by R¹¹.

R₃ is oxadiazole which is unsubstituted or substituted by one methyl.

R₃ is benzyl which is unsubstituted or substituted one or more times by R¹¹.

R₃ is benzyl.

R₃ is methyl, ethyl, propyl, isopropyl, butyl, sec-butyl, or tert-butyl.

R₃ is methyl.

R₃′ is H or C₁₋₁₂ alkyl which is unsubstituted or substituted one or more times by R₁₀.

R₃′ is H or C₁₋₆ alkyl which is unsubstituted or substituted one or more times by R₁₀.

R₃′ is H, methyl, ethyl, propyl, isopropyl, butyl, sec-butyl, tert-butyl, cyclopropyl, cyclobutyl, cyclopentyl, or cyclohexyl.

R₃′ is methyl, ethyl, propyl, isopropyl, butyl, sec-butyl, or tert-butyl.

R₃′ is methyl.

R₃ and R₃′ are each independently H, methyl, ethyl, propyl, isopropyl, butyl, sec-butyl, tert-butyl, cyclopropyl, cyclobutyl, cyclopentyl, or cyclohexyl.

One of R₃ and R₃′ is H and the other is methyl, ethyl, propyl, isopropyl, butyl, sec-butyl, tert-butyl, cyclopropyl, cyclobutyl, cyclopentyl, or cyclohexyl.

R₃ and R₃′ are each independently methyl, ethyl, propyl, isopropyl, butyl, sec-butyl, or tert-butyl.

R₃ is H.

R₃′ is H.

R₃ and R₃′ are both H.

R₃ and R₃′ can also be taken together to form a 5-6 member heteroaryl which is unsubstituted or substituted one or more times by R₁₁, or a 5-6 member heterocycle which is unsubstituted or substituted one or more times by R₁₂.

R₃ and R₃′ can also be taken together to form a piperidyl, a piperazinyl, or a morpholinyl which is unsubstituted or substituted one or more times by R₁₁.

R₃ and R₃′ can also be taken together to form a piperazinyl which is unsubstituted or substituted one or more times by R₁₁.

R₄ is C₁₋₁₂ alkyl which is unsubstituted or substituted one or more times by R₁₀, C₂₋₁₂ alkenyl which is unsubstituted or substituted one or more times by R₁₀, C₂₋₁₂ alkynyl which is unsubstituted or substituted one or more times by R₁₀, C₆₋₁₄ aryl which is unsubstituted or substituted one or more times by R₁₁, C₇₋₁₆ aralkyl which is unsubstituted or substituted one or more times by R₁₁, 5-12 member heteroaryl which is unsubstituted or substituted one or more times by R₁₁, 6-18 member heteroaralkyl which is unsubstituted or substituted one or more times by R₁₁, 3-12 member heterocycle which is unsubstituted or substituted one or more times by R₁₂, or 4-18 member heterocycle-alkyl which is unsubstituted or substituted one or more times by R₁₂.

R₄ is C₁₋₁₂ alkyl which is unsubstituted or substituted one or more times by R₁₀, C₆ aryl which is unsubstituted or substituted one or more times by R₁₁, C₇₋₉ aralkyl which is unsubstituted or substituted one or more times by R₁₁, 5-6 member heteroaryl which is unsubstituted or substituted one or more times by R₁₁, 7-8 member heteroaralkyl which is unsubstituted or substituted one or more times by R₁₁, 5-6 member heterocycle which is unsubstituted or substituted one or more times by R₁₂, or 7-8 member heterocycle-alkyl which is unsubstituted or substituted one or more times by R₁₂.

R₄ is C₁₋₆ alkyl which is unsubstituted or substituted one or more times by R₁₀, phenyl which is unsubstituted or substituted one or more times by R₁₁, benzyl which is unsubstituted or substituted one or more times by R₁₁, 5-6 member heteroaryl which is unsubstituted or substituted one or more times by R₁₁, 7-8 member heteroaralkyl which is unsubstituted or substituted one or more times by R₁₁, 5-6 member heterocycle which is unsubstituted or substituted one or more times by R₁₂, or 7-8 member heterocycle-alkyl which is unsubstituted or substituted one or more times by R₁₂.

R₄ is C₁₋₆ alkyl which is unsubstituted or substituted one or more times by R₁₀, phenyl which is unsubstituted or substituted one or more times by R₁₁, benzyl which is unsubstituted or substituted one or more times by R₁₁, or 6 member heteroaryl which is unsubstituted or substituted one or more times by R₁₁.

R₄ is C₁₋₆ alkyl which is unsubstituted or substituted one or more times by R₁₀, phenyl which is unsubstituted or substituted one or more times by R₁₁, benzyl which is unsubstituted or substituted one or more times by R₁₁, or pyridyl which is unsubstituted or substituted one or more times by R₁₁.

R₄ is 7-8 member heterocycle-alkyl which is unsubstituted or substituted one or more times by R₁₂.

R₄ is piperidynyl, piperazinyl, tetrahydropyranyl, and pyrrolidinyl which are unsubstituted or substituted one or more times by R₁₂.

R₄ is oxadiazolyl, thiazolyl, pyridinyl, oxadiazolyl and pyrazolyl, which are unsubstituted or substituted one or more times by R₁₁.

R₄ is heterocycle-alkyl which is pyrrolidinyl ethyl.

R₄ is heterocycle-alkyl which is piperidinyl methyl.

R₄ is 5-6 member heteroaryl which is unsubstituted or substituted one or more times by R¹¹.

R₄ is oxadiazole which is unsubstituted or substituted one or more times by R¹¹.

R₄ is oxadiazole which is unsubstituted or substituted by one methyl.

R₄ is methyl, ethyl, propyl, isopropyl, butyl, sec-butyl, tert-butyl, cyclopropyl, cyclobutyl, cyclopentyl, or cyclohexyl.

R₄ is phenyl which is unsubstituted or substituted one or more times by R₁₁.

R₄ is phenyl.

R₄ is benzyl which is unsubstituted or substituted one or more times by R₁₁.

R₄ is benzyl.

R₄ is pyridyl which is unsubstituted or substituted one or more times by R₁₁.

R₄ is pyridyl.

R₅ is piperidynyl, piperazinyl, tetrahydropyranyl, and pyrrolidinyl which are unsubstituted or substituted one or more times by R₁₂.

R₅ is oxadiazolyl, thiazolyl, pyridinyl, oxadiazolyl and pyrazolyl, which are unsubstituted or substituted one or more times by R₁₁.

R₅ is phenyl which is unsubstituted or substituted one or more times by R₁₁.

R₅ is phenyl.

R₅ is benzyl which is unsubstituted or substituted one or more times by R₁₁.

R₅ is benzyl.

R₅ is pyridyl which is unsubstituted or substituted one or more times by R₁₁.

R₅ is pyridyl.

R₅ is C₁₋₁₂ alkyl which is unsubstituted or substituted one or more times by R₁₀.

R₅ is C₁₋₆ alkyl which is unsubstituted or substituted one or more times by R₁₀.

R₅ is methyl, ethyl, propyl, isopropyl, butyl, sec-butyl, tert-butyl, cyclopropyl, cyclobutyl, cyclopentyl, or cyclohexyl.

R₅ is methyl, ethyl, propyl, isopropyl, butyl, sec-butyl, or tert-butyl.

R₆ is C₁₋₁₂ alkyl which is unsubstituted or substituted one or more times by R₁₀, C₂₋₁₂ alkenyl which is unsubstituted or substituted one or more times by R₁₀, C₂₋₁₂ alkynyl which is unsubstituted or substituted one or more times by R₁₀, C₆₋₁₄ aryl which is unsubstituted or substituted one or more times by R₁₁, C₇₋₁₆ aralkyl which is unsubstituted or substituted one or more times by R₁₁, 5-12 member heteroaryl which is unsubstituted or substituted one or more times by R₁₁, 6-18 member heteroaralkyl which is unsubstituted or substituted one or more times by R₁₁, 3-12 member heterocycle which is unsubstituted or substituted one or more times by R₁₂, or 4-18 member heterocycle-alkyl which is unsubstituted or substituted one or more times by R₁₂.

R₆ is C₁₋₁₂ alkyl which is unsubstituted or substituted one or more times by R₁₀, C₆ aryl which is unsubstituted or substituted one or more times by R₁₁, C₇₋₉ aralkyl which is unsubstituted or substituted one or more times by R₁₁, 5-6 member heteroaryl which is unsubstituted or substituted one or more times by R₁₁, 7-8 member heteroaralkyl which is unsubstituted or substituted one or more times by R₁₁, 5-6 member heterocycle which is unsubstituted or substituted one or more times by R₁₂, or 7-8 member heterocycle-alkyl which is unsubstituted or substituted one or more times by R₁₂.

R₆ is C₁₋₆ alkyl which is unsubstituted or substituted one or more times by R₁₀, phenyl which is unsubstituted or substituted one or more times by R₁₁, benzyl which is unsubstituted or substituted one or more times by R₁₁, 5-6 member heteroaryl which is unsubstituted or substituted one or more times by R₁₁, 7-8 member heteroaralkyl which is unsubstituted or substituted one or more times by R₁₁, 5-6 member heterocycle which is unsubstituted or substituted one or more times by R₁₂, or 7-8 member heterocycle-alkyl which is unsubstituted or substituted one or more times by R₁₂.

R₆ is C₁₋₆ alkyl which is unsubstituted or substituted one or more times by R₁₀, phenyl which is unsubstituted or substituted one or more times by R₁₁, benzyl which is unsubstituted or substituted one or more times by R₁₁, or 6 member heteroaryl which is unsubstituted or substituted one or more times by R₁₁.

R₆ is C₁₋₆ alkyl which is unsubstituted or substituted one or more times by R₁₀, phenyl which is unsubstituted or substituted one or more times by R₁₁, benzyl which is unsubstituted or substituted one or more times by R₁₁, or pyridyl which is unsubstituted or substituted one or more times by R₁₁.

R₆ is C₁₋₁₂ alkyl which is unsubstituted or substituted one or more times by R₁₀.

R₆ is C₁₋₆ alkyl which is unsubstituted or substituted one or more times by R₁₀.

R₆ is methyl, ethyl, propyl, isopropyl, butyl, sec.-butyl, tert.-butyl, cyclopropyl, cyclobutyl, cyclopentyl, or cyclohexyl.

R₆ is methyl, ethyl, propyl, isopropyl, butyl, sec.-butyl, or tert.-butyl.

R₆ is methyl.

R₆ is phenyl which is unsubstituted or substituted one or more times by R₁₁.

R₆ is phenyl.

R₆ is benzyl which is unsubstituted or substituted one or more times by R₁₁.

R₆ is benzyl.

R₆ is pyridyl which is unsubstituted or substituted one or more times by R₁₁.

R₆ is pyridyl.

R₆ is piperidynyl, piperazinyl, tetrahydropyranyl, and pyrrolidinyl which are unsubstituted or substituted one or more times by R₁₂.

R₆ is oxadiazolyl, thiazolyl, pyridinyl, oxadiazolyl and pyrazolyl, which are unsubstituted or substituted one or more times by R₁₁.

R₁₀ is halogen, oxo, C₁₋₆ alkoxy, —NH₂, —NH(C₁₋₄ alkyl), —N(C₁₋₄ alkyl)₂, —CONH₂, —CONH(C₁₋₄ alkyl), —CON(C₁₋₄ alkyl)₂, —NHCOH, —N(C₁₋₄ alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —N(C₁₋₄ alkyl)CONHC₁₋₄ alkyl, —N(C₁₋₄ alkyl)CON(C₁₋₄ alkyl)₂, —NHCON(C₁₋₄ alkyl)₂, —C(O)H, —C(O)C₁₋₄ alkyl, carboxy, —C(O)OC₁₋₄ alkyl, —C(NOH)C₁₋₄ alkyl, —C(NOH)H, hydroxyl, nitro, azido, cyano, —S(O)₀₋₂H, —S(O)₀₋₂C₁₋₄ alkyl, —SO₂NH₂, —SO₂NH(C₁₋₄ alkyl), —SO₂N(C₁₋₄ alkyl)₂, —N(C₁₋₄ alkyl)SO₂C₁₋₄ alkyl, —NHSO₂C₁₋₄ alkyl, or —P(O)(OH)₂.

R₁₀ is halogen, oxo, —NH₂, —NH(C₁₋₄ alkyl), —N(C₁₋₄ alkyl)₂, —CONH₂, —CONH(C₁₋₄ alkyl), —CON(C₁₋₄ alkyl)₂, —NHCOH, —N(C₁₋₄ alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —C(O)H, —C(O)C₁₋₄ alkyl, carboxy, —C(O)OC₁₋₄ alkyl, hydroxyl, C₁₋₄ alkoxy, nitro, nitroso, azido, or cyano.

R₁₀ is halogen, oxo, —NH₂, —NH(C₁₋₄ alkyl), —N(C₁₋₄ alkyl)₂, —CONH₂, —CONH(C₁₋₄ alkyl), —CON(C₁₋₄ alkyl)₂, —NHCOH, —N(C₁₋₄ alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —C(O)H, —C(O)C₁₋₄ alkyl, carboxy, —C(O)OC₁₋₄ alkyl, hydroxyl, C₁₋₄ alkoxy, nitro, azido, or cyano.

R₁₀ is halogen, oxo, —NH₂, —NH(C₁₋₄ alkyl), —N(C₁₋₄ alkyl)₂, —CONH₂, —CONH(C₁₋₄ alkyl), —CON(C₁₋₄ alkyl)₂, —NHCOH, —N(C₁₋₄ alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —C(O)H, —C(O)C₁₋₄ alkyl, carboxy, —C(O)OC₁₋₄ alkyl, hydroxyl, or C₁₋₄ alkoxy.

R¹⁰ is halogen, oxo, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄ alkyl, —NHCOC₁₋₄ alkyl, carboxy, —C(O)OC₁₋₄ alkyl, hydroxyl, C₁₋₄ alkoxy, or cyano.

R¹⁰ is halogen, hydroxyl, or C₁₋₃ alkoxy.

R₁₁ is halogen, C₁₋₆ alkyl, halogenated C₁₋₆ alkyl, C₂₋₆ alkenyl, C₂₋₆ alkynyl, C₁₋₆ alkoxy, —NH₂, —NH(C₁₋₄ alkyl), —N(C₁₋₄ alkyl)₂, —CONH₂, —CONH(C₁₋₄ alkyl), —CON(C₁₋₄ alkyl)₂, —NHCOH, —N(C₁₋₄ alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —N(C₁₋₄alkyl)CONHC₁₋₄ alkyl, —N(C₁₋₄ alkyl)CON(C₁₋₄ alkyl)₂, —NHCON(C₁₋₄ alkyl)₂, —C(O)H, —C(O)C₁₋₄ alkyl, carboxy, —C(O)O C₁₋₄ alkyl, —C(NOH)C₁₋₄ alkyl, —C(NOH)H, hydroxyl, nitro, azido, cyano, —S(O)₀₋₂H, —S(O)₀₋₂C₁₋₄ alkyl, —SO₂NH₂, —SO₂NH(C₁₋₄ alkyl), —SO₂N(C₁₋₄ alkyl)₂, —N(C₁₋₄ alkyl)SO₂C₁₋₄ alkyl, —NHSO₂C₁₋₄ alkyl, or —P(O)(OH)₂.

R₁₁ is halogen, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —NHCOH, —N(C₁₋₄ alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —C(O)H, —C(O)C₁₋₄ alkyl, carboxy, —C(O)OC₁₋₄ alkyl, hydroxyl, C₁₋₆ alkoxy, nitro, nitroso, azido, or cyano.

R₁₁ is halogen, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —NHCOH, —N(C₁₋₄ alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —C(O)H, —C(O)C₁₋₄ alkyl, carboxy, —C(O)OC₁₋₄ alkyl, hydroxyl, C₁₋₆ alkoxy, nitro, azido, or cyano.

R₁₁ is halogen, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —NHCOH, —N(C₁₋₄alkyl)COH, —N(C₁₋₄alkyl)COC₁₋₄alkyl, —NHCOC₁₋₄alkyl, —NHCOOC₁₋₄ alkyl, —NHCONHC₁₋₄ alkyl, —C(O)H, —C(O)C₁₋₄ alkyl, carboxy, —C(O)OC₁₋₄ alkyl, hydroxyl, or C₁₋₆ alkoxy.

R¹¹ is halogen, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄ alkyl, —NHCOC₁₋₄ alkyl, carboxy, —C(O)OC₁₋₄ alkyl, hydroxyl, or C₁₋₆ alkoxy.

R¹¹ is halogen, C₁₋₃ alkyl, halogenated C₁₋₃ alkyl, hydroxyl, or C₁₋₃ alkoxy.

R₁₂ is halogen, oxo, C₁₋₆ alkyl, halogenated C₁₋₆ alkyl, C₂₋₆ alkenyl, C₂₋₆ alkynyl, C₁₋₆ alkoxy, —NH₂, —NH(C₁₋₄ alkyl), —N(C₁₋₄ alkyl)₂, —CONH₂, —CONH(C₁₋₄ alkyl), —CON(C₁₋₄ alkyl)₂, —NHCOH, —N(C₁₋₄ alkyl)COH, —N(C₁₋₄alkyl)COC₁₋₄, alkyl, —NHCOC₁₋₄ alkyl, —NHCOOC₁₋₄ alkyl, —NHCONHC₁₋₄ alkyl, —N(C₁₋₄alkyl)CONHC₁₋₄ alkyl, —N(C₁₋₄ alkyl)CON(C₁₋₄alkyl)₂, —NHCON(C₁₋₄ alkyl)₂, —C(O)H, —C(O)C₁₋₄alkyl, carboxy, —C(O)OC₁₋₄ alkyl, —C(NOH)C₁₋₄ alkyl, —C(NOH)H, hydroxyl, nitro, azido, cyano, —S(O)₀₋₂H, —S(O)₀₋₂C₁₋₄ alkyl, —SO₂NH₂, —SO₂NH(C₁₋₄ alkyl), —SO₂N(C₁₋₄ alkyl)₂, —N(C₁₋₄ alkyl)SO₂C₁₋₄ alkyl, —NHSO₂C₁₋₄ alkyl, or —P(O)(OH)₂.

R₁₂ is halogen, oxo, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄alkyl)₂, —CONH₂, —CONH(C₁₋₄ alkyl), —CON(C₁₋₄ alkyl)₂, —NHCOH, —N(C₁₋₄alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —C(O)H, —C(O)C₁₋₄ alkyl, carboxy, —C(O)OC₁₋₄ alkyl, hydroxyl, C₁₋₆ alkoxy, nitro, nitroso, azido, or cyano.

R₁₂ is halogen, oxo, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄alkyl)₂, —CONH₂, —CONH(C₁₋₄ alkyl), —CON(C₁₋₄ alkyl)₂, —NHCOH, —N(C₁₋₄alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —C(O)H, —C(O)C₁₋₄ alkyl, carboxy, —C(O)OC₁₋₄ alkyl, hydroxyl, C₁₋₆ alkoxy, nitro, azido, or cyano.

R₁₂ is halogen, oxo, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —NHCOH, —N(C₁₋₄alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —C(O)H, —C(O)C₁₋₄ alkyl, carboxy, —C(O)OC₁₋₄ alkyl, hydroxyl, or C₁₋₆ alkoxy.

R¹² is halogen, oxo, C₁₋₆ alkyl, halogenated C₁₋₆ alkyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄ alkyl, —NHCOC₁₋₄ alkyl, carboxy, —C(O)OC₁₋₄ alkyl, hydroxyl, or C₁₋₆ alkoxy.

R¹² is halogen, oxo, C₁₋₃ alkyl, halogenated C₁₋₃ alkyl, hydroxyl, or C₁₋₃ alkoxy.

In a further embodiment, the present invention relates to a compound of formula (II) and (IIa):

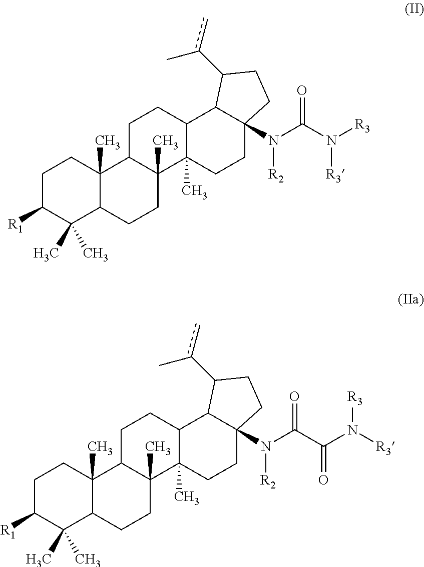

or a pharmaceutically acceptable salt thereof, wherein R₁, R₂, R₃, and R₃′ are defined above.

In a further embodiment, the present invention relates to a compound of formula (II):

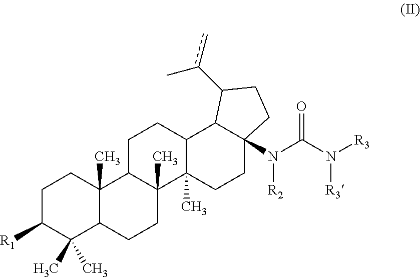

or a pharmaceutically acceptable salt thereof, wherein R₁, R₂, R₃, and R₃′ are defined above.

In a further embodiment, the present invention relates to a compound of formula (III):

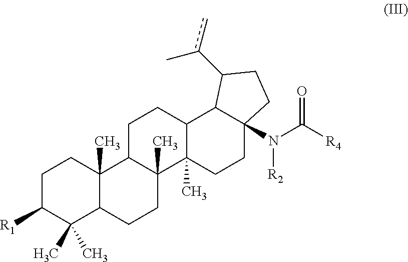

or a pharmaceutically acceptable salt thereof, wherein R₁, R₂, and R₄ are defined above.

In a further embodiment, the present invention relates to a compound of formula (IV):

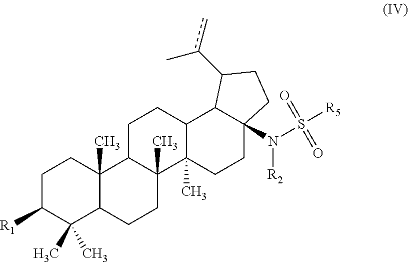

or a pharmaceutically acceptable salt thereof, wherein R₁, R₂, and R₅ are defined above.

In a further embodiment, the present invention relates to a compound of formula (V), (Va), and (Vb):

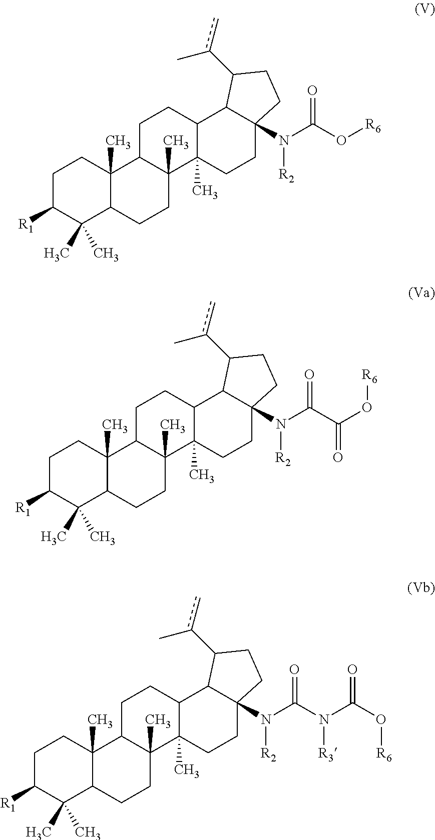

or a pharmaceutically acceptable salt thereof, wherein R₁, R₂, R₃′ and R₆ are defined above.

In a further embodiment, the present invention relates to a compound of formula (V):

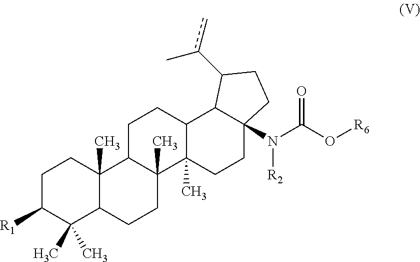

or a pharmaceutically acceptable salt thereof, wherein R₁, R₂, and R₆ are defined above.

In a further embodiment, the present invention relates to a compound of formula (VI) and (Via):

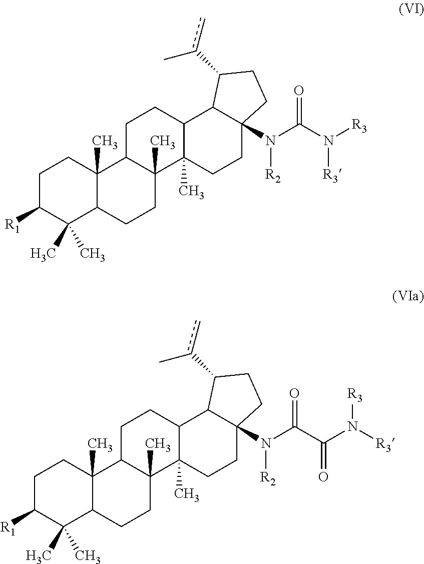

or a pharmaceutically acceptable salt thereof, wherein R₁, R₂, R₃, and R₃′ are defined above.

In a further embodiment, the present invention relates to a compound of formula (VI):

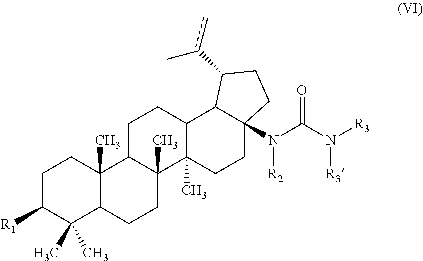

or a pharmaceutically acceptable salt thereof, wherein R₁, R₂, R₃, and R₃′ are defined above.

In a further embodiment, the present invention relates to a compound of formula (VII):

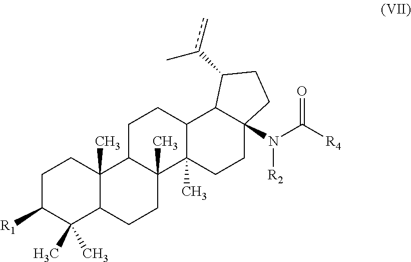

or a pharmaceutically acceptable salt thereof, wherein R₁, R₂, and R₄ are defined above.

In a further embodiment, the present invention relates to a compound of formula (VIII):

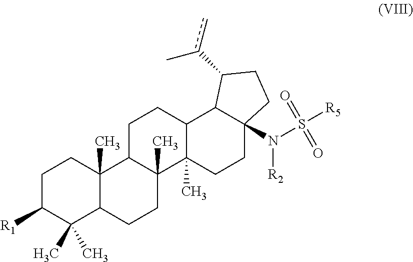

or a pharmaceutically acceptable salt thereof, wherein R₁, R₂, and R₅ are defined above.

In a further embodiment, the present invention relates to a compound of formula (IX), (IXa), and (IXb):

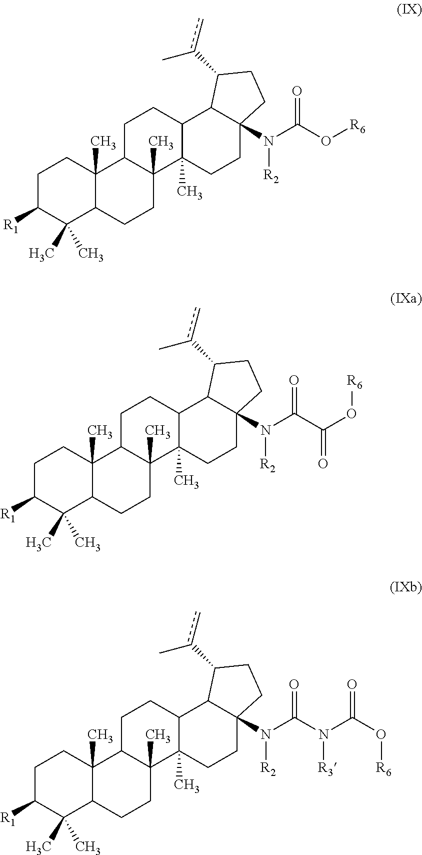

or a pharmaceutically acceptable salt thereof, wherein R₁, R₂, R₃′ and R₆ are defined above.

In a further embodiment, the present invention relates to a compound of formula (IX):

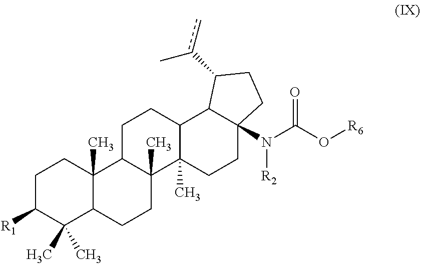

or a pharmaceutically acceptable salt thereof, wherein R₁, R₂, and R₆ are defined above.

In a further embodiment, the present invention relates to a compound of formula (X) and (Xa):

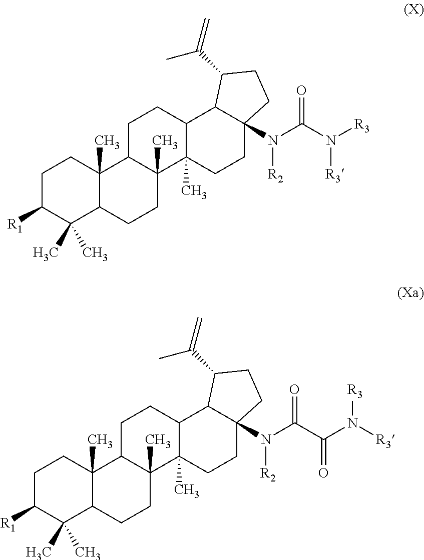

or a pharmaceutically acceptable salt thereof, wherein R₁, R₂, R₃, and R₃′ are defined above.

In a further embodiment, the present invention relates to a compound of formula (X):

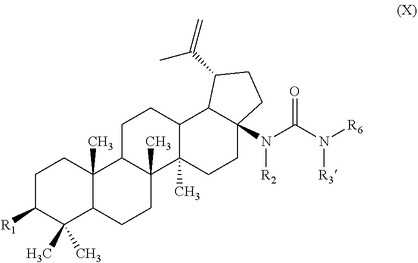

or a pharmaceutically acceptable salt thereof, wherein R₁, R₂, R₃, and R₃′ are defined above.

In a further embodiment, the present invention relates to a compound of formula (XI):

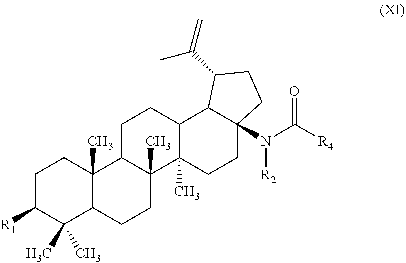

or a pharmaceutically acceptable salt thereof, wherein R₁, R₂, and R₄ are defined above.

In a further embodiment, the present invention relates to a compound of formula (XII):

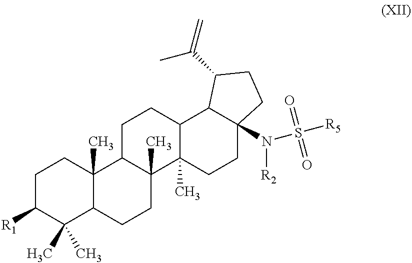

or a pharmaceutically acceptable salt thereof, wherein R₁, R₂, and R₅ are defined above.

In a further embodiment, the present invention relates to a compound of formula (XIII), (XIIIa), and (XIIIb):

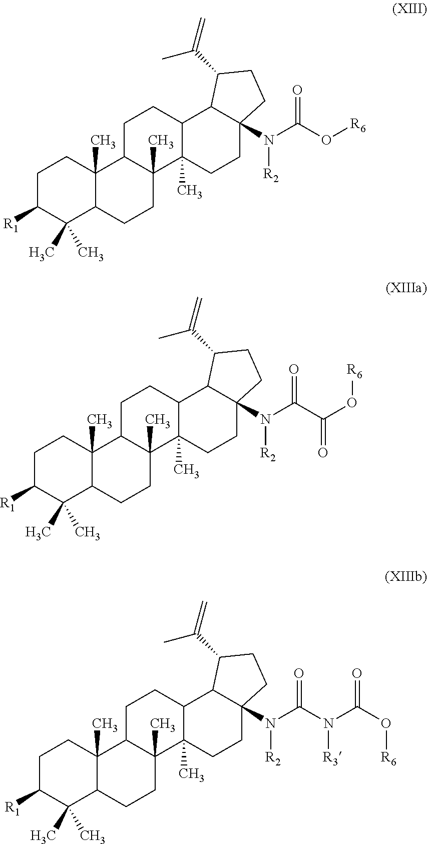

or a pharmaceutically acceptable salt thereof, wherein R₁, R₂, R₃′ and R₆ are defined above.

In a further embodiment, the present invention relates to a compound of formula (XIII):

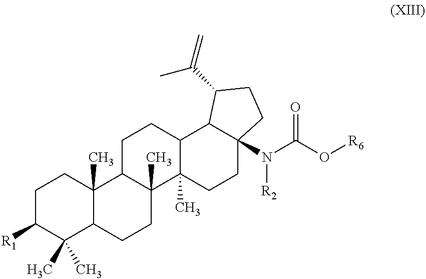

or a pharmaceutically acceptable salt thereof, wherein R₁, R₂, and R₆ are defined above.

In further a embodiment, the compounds of the invention are represented by formula (I) to (XIIIb) wherein:

R₁ is O-succinyl, O-glutaryl, O-3′-methylglutaryl, O-3′-methylsuccinyl, O-3′,3′-dimethylsuccinyl, O-3′,3′-dimethylglutaryl, O-2′,2′-dimethylmalonyl, O-2′,3′-dihydroxysuccinyl, O-2′,2′,3′,3′-tetramethylsuccinyl, or O-2′,2′-dimethylsuccinyl;

R₂ is H, methyl, ethyl, propyl, isopropyl, butyl, sec-butyl, tert-butyl, cyclopropyl, cyclobutyl, cyclopentyl, or cyclohexyl;

R₃′ is H, methyl, ethyl, propyl, isopropyl, butyl, sec-butyl, tert-butyl, cyclopropyl, cyclobutyl, cyclopentyl, or cyclohexyl;

R₃ and R₃′ can also be taken together to form a 5-6 member heteroaryl which is unsubstituted or substituted one or more times by R₁₁, or a 5-6 member heterocycle which is unsubstituted or substituted one or more times by R₁₂;

R₃, R₄, R₅ and R₆ are each independently C₁₋₆ alkyl which is unsubstituted or substituted one or more times by R¹⁰, phenyl which is unsubstituted or substituted one or more times by R¹¹, benzyl which is unsubstituted or substituted one or more times by R¹¹, 5-6 member heteroaryl which is unsubstituted or substituted one or more times by R¹¹, 7-8 member heteroaralkyl which is unsubstituted or substituted one or more times by R¹¹, 5-6 member heterocycle which is unsubstituted or substituted one or more times by R¹², or 7-8 member heterocycle-alkyl which is unsubstituted or substituted one or more times by R¹²;

R¹⁰ is halogen, oxo, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄ alkyl, —NHCOC₁₋₄ alkyl, carboxy, —C(O)OC₁₋₄ alkyl, hydroxyl, C₁₋₄ alkoxy, or cyano;

R¹¹ is halogen, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄ alkyl, —NHCOC₁₋₄ alkyl, carboxy, —C(O)OC₁₋₄ alkyl, hydroxyl, or C₁₋₆ alkoxy; and

R¹² is halogen, oxo, C₁₋₆ alkyl, halogenated C₁₋₆ alkyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄ alkyl, —NHCOC₁₋₄ alkyl, carboxy, —C(O)OC₁₋₄ alkyl, hydroxyl, or C₁₋₆ alkoxy.

In a further embodiment, the compounds of the invention are represented by formula (I) to (XIIIb) wherein:

R₁ is O-3′,3′-dimethylsuccinyl;

R₂ is H;

R₃′ is H or methyl;

R₃ and R₃′ can also be taken together to form a 5-6 member heteroaryl which is unsubstituted or substituted one or more times by R₁₁, or a 5-6 member heterocycle which is unsubstituted or substituted one or more times by R₁₂;

R₃, R₄, R₅ and R₆ are each independently C₁₋₆ alkyl which is unsubstituted or substituted one or more times by R¹⁰, phenyl which is unsubstituted or substituted one or more times by R¹¹, benzyl which is unsubstituted or substituted one or more times by R¹¹, 5-6 member heteroaryl which is unsubstituted or substituted one or more times by R¹¹, 7-8 member heteroaralkyl which is unsubstituted or substituted one or more times by R¹¹, 5-6 member heterocycle which is unsubstituted or substituted one or more times by R¹², or 7-8 member heterocycle-alkyl which is unsubstituted or substituted one or more times by R¹²;

R¹⁰ is halogen, oxo, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄ alkyl, —NHCOC₁₋₄ alkyl, carboxy, —C(O)OC₁₋₄ alkyl, hydroxyl, C₁₋₄ alkoxy, or cyano;

R¹¹ is halogen, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄ alkyl, —NHCOC₁₋₄ alkyl, carboxy, —C(O)OC₁₋₄ alkyl, hydroxyl, or C₁₋₆ alkoxy; and

R¹² is halogen, oxo, C₁₋₆ alkyl, halogenated C₁₋₆ alkyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄ alkyl, —NHCOC₁₋₄ alkyl, carboxy, —C(O)OC₁₋₄ alkyl, hydroxyl, or C₁₋₆ alkoxy.

In a further embodiment, the compounds of the invention are represented by formula (I) to (Ic) wherein:

R₁ is O-succinyl, O-glutaryl, O-3′-methylglutaryl, O-3′-methylsuccinyl, O-3′,3′-dimethylsuccinyl, O-3′,3′-dimethylglutaryl, O-2′,2′-dimethylmalonyl, O-2′,3′-dihydroxysuccinyl, O-2′,2′,3′,3′-tetramethylsuccinyl, or O-2′,2′-dimethylsuccinyl;

R₂ is H, methyl, ethyl, propyl, isopropyl, butyl, sec-butyl, tert-butyl, cyclopropyl, cyclobutyl, cyclopentyl, or cyclohexyl;

R₃ and R₃′ are each independently H, methyl, ethyl, propyl, isopropyl, butyl, sec-butyl, or tert-butyl or R₃ and R₃′ taken together form a piperidyl, a piperazinyl, or a morpholinyl which is unsubstituted or substituted one or more times by R₁₁;

R₄ is C₁₋₆ alkyl which is unsubstituted or substituted one or more times by R₁₀, phenyl which is unsubstituted or substituted one or more times by R₁₁, benzyl which is unsubstituted or substituted one or more times by R₁₁, 5-6 member heteroaryl which is unsubstituted or substituted one or more times by R₁₁, 7-8 member heteroaralkyl which is unsubstituted or substituted one or more times by R₁₁, 5-6 member heterocycle which is unsubstituted or substituted one or more times by R₁₂, or 7-8 member heterocycle-alkyl which is unsubstituted or substituted one or more times by R₁₂;

R₅ is methyl, ethyl, propyl, isopropyl, butyl, sec-butyl, tert-butyl, cyclopropyl, cyclobutyl, cyclopentyl, or cyclohexyl;

R₆ is methyl, ethyl, propyl, isopropyl, butyl, sec-butyl, tert-butyl, cyclopropyl, cyclobutyl, cyclopentyl, or cyclohexyl;

R₁₀ is halogen, oxo, —NH₂, —NH(C₁₋₄ alkyl), —N(C₁₋₄ alkyl)₂, —CONH₂, —CONH(C₁₋₄ alkyl), —CON(C₁₋₄alkyl)₂, —NHCOH, —N(C₁₋₄ alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —C(O)H, —C(O)C₁₋₄ alkyl, carboxy, —C(O)OC₁₋₄ alkyl, hydroxyl, or C₁₋₄ alkoxy;

R₁₁ is halogen, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —NHCOH, —N(C₁₋₄ alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —C(O)H, —C(O)C₁₋₄ alkyl, carboxy, —C(O)OC₁₋₄ alkyl, hydroxyl, or C₁₋₆ alkoxy; and

R₁₂ is halogen, oxo, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —NHCOH, —N(C₁₋₄alkyl)COH, —N(C₁₋₄ alkyl)COC₁₋₄ alkyl, —NHCOC₁₋₄ alkyl, —NHCOOC₁₋₄ alkyl, —NHCONHC₁₋₄ alkyl, —C(O)H, —C(O)C₁₋₄ alkyl, carboxy, —C(O)OC₁₋₄ alkyl, hydroxyl, or C₁₋₆ alkoxy.

In a further embodiment, the compounds of the invention are represented by formula (I) to (Ic) wherein:

R₁ is O-3′,3′-dimethylsuccinyl;

R₂ is H;

R₃′ is H;

R₃ is methyl, ethyl, propyl, isopropyl, butyl, sec-butyl, or tert-butyl or R₃ and

R₃′ taken together form a piperidyl, a piperazinyl, or a morpholinyl which is unsubstituted or substituted one or more times by R₁₁;

R₄ is benzyl or methyl;

R₅ is methyl, ethyl, propyl, isopropyl, butyl, sec-butyl, tert-butyl, cyclopropyl, cyclobutyl, cyclopentyl, or cyclohexyl; and

R₆ is methyl, ethyl, propyl, isopropyl, butyl, sec-butyl, tert-butyl, cyclopropyl, cyclobutyl, cyclopentyl, or cyclohexyl.

In a further embodiment, the compounds of the invention are represented by formula (I) to (Ic), (II), (IIa), (VI), (Via), (X) or (Xa) wherein:

R₁ is O-3′,3′-dimethylsuccinyl;

R₂ is H;

R₃′ is H or methyl;

R₃ is C₁₋₁₂ alkyl which is unsubstituted or substituted one or more times by R¹⁰, C₂₋₁₂ alkenyl which is unsubstituted or substituted one or more times by R¹⁰, C₂₋₁₂ alkynyl which is unsubstituted or substituted one or more times by R¹⁰, C₆₋₁₄ aryl which is unsubstituted or substituted one or more times by R¹¹, C₇₋₁₆ aralkyl which is unsubstituted or substituted one or more times by R¹¹, 5-12 member heteroaryl which is unsubstituted or substituted one or more times by R¹¹, 6-18 member heteroaralkyl which is unsubstituted or substituted one or more times by R¹¹, 3-12 member heterocycle which is unsubstituted or substituted one or more times by R¹², or 4-18 member heterocycle-alkyl which is unsubstituted or substituted one or more times by R¹²;
R¹⁰ is halogen, oxo, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂—N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄ alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino;
R¹¹ is halogen, C₁₋₆ alkyl, halogenated C₁₋₆ alkyl, C₂₋₆ alkenyl, C₂₋₆ alkynyl, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino; and
R¹² is halogen, oxo, C₁₋₆ alkyl, halogenated C₁₋₆ alkyl, C₂₋₆ alkenyl, C₂₋₆ alkynyl, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino.

In a further embodiment, the compounds of the invention are represented by formula (I) to (Ic), (II), (IIa), (VI), (Via), (X) or (Xa) wherein:

R₁ is O-3′,3′-dimethylsuccinyl;

R₂ is H;

R₃′ is H or methyl;

R₃ is methyl, ethyl, propyl, isopropyl, butyl, sec-butyl, tert-butyl, oxadiazole, benzyl, or R₃ and R₃′ taken together form a piperidyl, a piperazinyl, or a morpholinyl which is unsubstituted or substituted one or more times by R₁₁; and

R¹¹ is halogen, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄ alkyl, —NHCOC₁₋₄ alkyl, carboxy, —C(O)OC₁₋₄ alkyl, hydroxyl, or C₁₋₆ alkoxy.

In a further embodiment, the compounds of the invention are represented by formula (I) to (Ic), (III), (VII) or (XI) wherein:

R₁ is O-3′,3′-dimethylsuccinyl;

R₂ is H;

R₄ is C₁₋₁₂ alkyl which is unsubstituted or substituted one or more times by R₁₀, C₂₋₁₂ alkenyl which is unsubstituted or substituted one or more times by R₁₀, C₂₋₁₂ alkynyl which is unsubstituted or substituted one or more times by R₁₀, C₆₋₁₄ aryl which is unsubstituted or substituted one or more times by R₁₁, C₇₋₁₆ aralkyl which is unsubstituted or substituted one or more times by R₁₁, 5-12 member heteroaryl which is unsubstituted or substituted one or more times by R₁₁, 6-18 member heteroaralkyl which is unsubstituted or substituted one or more times by R₁₁, 3-12 member heterocycle which is unsubstituted or substituted one or more times by R₁₂, or 4-18 member heterocycle-alkyl which is unsubstituted or substituted one or more times by R₁₂;
R¹⁰ is halogen, oxo, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —SO(O)₀₋₃H, —S(O)₀₋₃C₁₋₄ alkyl, —SO₂NH₂, —SO₂NH(C₁₋₄ alkyl), —SO₂N(C₁₋₄ alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino;
R¹¹ is halogen, C₁₋₆ alkyl, halogenated C₁₋₆ alkyl, C₂₋₆ alkenyl, C₂₋₆ alkynyl, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino; and
R¹² is halogen, oxo, C₁₋₆ alkyl, halogenated C₁₋₆ alkyl, C₂₋₆ alkenyl, C₂₋₆ alkynyl, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino.

In a further embodiment, the compounds of the invention are represented by formula (I) to (Ic), (III), (VII) or (XI) wherein:

R₁ is O-3′,3′-dimethylsuccinyl;

R₂ is H;

R₄ is methyl, ethyl, propyl, isopropyl, butyl, sec-butyl, tert-butyl, cyclopropyl, cyclobutyl, cyclopentyl, cyclohexyl, pyrrolidinyl ethyl, piperidinyl methyl, oxadiazole, phenyl, benzyl, or pyridiyl which is unsubstituted or substituted one or more times by R₁₁;

R¹¹ is halogen, C₁₋₆ alkyl, halogenated C₁₋₆ alkyl, C₂₋₆ alkenyl, C₂₋₆ alkynyl, —NH₂, —NH(C₁₋₄ alkyl), —N(C₁₋₄ alkyl)₂, —CONH₂, —CONH(C₁₋₄ alkyl), —CON(C₁₋₄ alkyl)₂, —N(C₁₋₄ alkyl)COC₁₋₄ alkyl, —NHCOC₁₋₄ alkyl, carboxy, —C(O)OC₁₋₄ alkyl, hydroxyl, or C₁₋₆ alkoxy.

In a further embodiment, the compounds of the invention are represented by formula (I) to (Ic), (V) to (Vb), (IX) to (IXb), or (XIII) to (XIIIb) wherein:

R₁ is O-3′,3′-dimethylsuccinyl;

R₂ is H;

R₃′ is H or methyl;

R₆ is C₁₋₁₂ alkyl which is unsubstituted or substituted one or more times by R₁₀, C₂₋₁₂ alkenyl which is unsubstituted or substituted one or more times by R₁₀, C₂₋₁₂ alkynyl which is unsubstituted or substituted one or more times by R₁₀, C₆₋₁₄ aryl which is unsubstituted or substituted one or more times by R₁₁, C₇₋₁₆ aralkyl which is unsubstituted or substituted one or more times by R₁₁, 5-12 member heteroaryl which is unsubstituted or substituted one or more times by R₁₁, 6-18 member heteroaralkyl which is unsubstituted or substituted one or more times by R₁₁, 3-12 member heterocycle which is unsubstituted or substituted one or more times by R₁₂, or 4-18 member heterocycle-alkyl which is unsubstituted or substituted one or more times by R₁₂;
R¹⁰ is halogen, oxo, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄ alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino;
R¹¹ is halogen, C₁₋₆ alkyl, halogenated C₁₋₆ alkyl, C₂₋₆ alkenyl, C₂₋₆ alkynyl, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino; and
R¹² is halogen, oxo, C₁₋₆ alkyl, halogenated C₁₋₆ alkyl, C₂₋₆ alkenyl, C₂₋₆ alkynyl, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino.

In a further embodiment, the compounds of the invention are represented by formula (I) to (Ic), (V) to (Vb), (IX) to (IXb), or (XIII) to (XIIIb) wherein:

R₁ is O-3′,3′-dimethylsuccinyl;

R₂ is H;

R₃′ is H or methyl; and

R₆ is methyl, ethyl, propyl, isopropyl, butyl, sec-butyl, tert-butyl, cyclopropyl, cyclobutyl, cyclopentyl, or cyclohexyl.

In a further embodiment, Ther is provided an intermediate represented by formula (XIV):

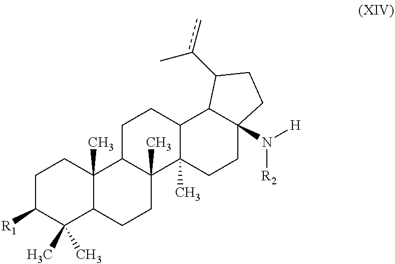

Wherein R₁ and R₂ are as defined above.

It will be appreciated by those skilled in the art that the compounds in accordance with the present invention can exists as stereoisomers, for example, optical (+ and −), geometrical (cis and trans) and conformational isomers (axial and equatorial). All such stereoisomers are included in the scope of the present invention.

It will be appreciated by those skilled in the art that the compounds in accordance with the present invention can contain a chiral center. The compounds of formula may thus exist in the form of two different optical isomers (i.e. (+) or (−) enantiomers). All such enantiomers and mixtures thereof including racemic mixtures are included within the scope of the invention. The single optical isomer or enantiomer can be obtained by methods well known in the art, such as chiral HPLC, enzymatic resolution and chiral auxiliary.

In one embodiment, the compounds of the present invention are provided in the form of a single enantiomer at least 95%, at least 97% and at least 99% free of the corresponding enantiomer.

In a further embodiment the compound of the present invention are in the form of the (+) enantiomer at least 95% free of the corresponding (−) enantiomer.

In a further embodiment the compound of the present invention are in the form of the (+) enantiomer at least 97% free of the corresponding (−) enantiomer.

In a further embodiment the compound of the present invention are in the form of the (+) enantiomer at least 99% free of the corresponding (−) enantiomer.

In a further embodiment, the compounds of the present invention are in the form of the (−) enantiomer at least 95% free of the corresponding (+) enantiomer.

In a further embodiment the compound of the present invention are in the form of the (−) enantiomer at least 97% free of the corresponding (+) enantiomer.

In a further embodiment the compound of the present invention are in the form of the (−) enantiomer at least 99% free of the corresponding (+) enantiomer.

There is also provided pharmaceutically acceptable salts of the compounds of the present invention. By the term pharmaceutically acceptable salts of compounds are meant those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toluene-p-sulphonic, tartaric, acetic, trifluoroacetic, citric, methanesulphonic, formic, benzoic, malonic, naphthalene-2-sulphonic and benzenesulphonic acids. Other acids such as oxalic, while not themselves pharmaceutically acceptable, may be useful as intermediates in obtaining the compounds of the invention and their pharmaceutically acceptable acid addition salts.

Salts derived from amino acids are also included (e.g. L-arginine, L-Lysine).

Salts derived from appropriate bases include alkali metals (e.g. sodium, lithium, potassium), alkaline earth metals (e.g. calcium, magnesium), ammonium, NR₄₊ (where R is C₁₋₄ alkyl) salts, choline, meglumine and tromethamine.

A reference hereinafter to a compound according to the invention includes that compound and its pharmaceutically acceptable salts.

In one embodiment of the invention, the pharmaceutically acceptable salt is a hydrochloride salt.

In one embodiment of the invention, the pharmaceutically acceptable salt is a sodium salt.

In one embodiment of the invention, the pharmaceutically acceptable salt is a lithium salt.

In one embodiment of the invention, the pharmaceutically acceptable salt is a potassium salt.

In one embodiment of the invention, the pharmaceutically acceptable salt is a tromethamine salt.

In one embodiment of the invention, the pharmaceutically acceptable salt is an L-arginine salt.

It will be appreciated by those skilled in the art that the compounds in accordance with the present invention can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

It will further be appreciated by those skilled in the art that the compounds in accordance with the present invention can exist in different solvate forms, for example hydrates. Solvates of the compounds of the invention may also form when solvent molecules are incorporated into the crystalline lattice structure of the compound molecule during the crystallization process.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

The term “alkyl” represents a linear, branched or cyclic hydrocarbon moiety. The terms “alkenyl” and “alkynyl” represent a linear, branched or cyclic hydrocarbon moiety which has one or more double bonds or triple bonds in the chain. Examples of alkyl, alkenyl, and alkynyl groups include but are not limited to methyl, ethyl, propyl, isopropyl, butyl, isobutyl, sec-butyl, tert-butyl, pentyl, isopentyl, neopentyl, tert-pentyl, hexyl, isohexyl, neohexyl, allyl, vinyl, acetylenyl, ethylenyl, propenyl, isopropenyl, butenyl, isobutenyl, butadienyl, pentenyl, pentadienyl, hexenyl, hexadienyl, hexatrienyl, heptenyl, heptadienyl, heptatrienyl, octenyl, octadienyl, octatrienyl, octatetraenyl, propynyl, butynyl, pentynyl, hexynyl, cyclopropyl, cyclobutyl, cyclopentyl, cyclohexenyl, cyclohexdienyl and cyclohexyl.

Where indicated the “alkyl,” “alkenyl,” and “alkynyl” can be optionally substituted such as in the case of haloalkyls in which one or more hydrogen atom is replaced by a halogen, e.g., an alkylhalide. Examples of haloalkyls include but are not limited to trifluoromethyl, difluoromethyl, fluoromethyl, trichloromethyl, dichloromethyl, chloromethyl, trifluoroethyl, difluoroethyl, fluoroethyl, trichloroethyl, dichloroethyl, chloroethyl, chlorofluoromethyl, chlorodifluoromethyl, dichlorofluoroethyl. Aside from halogens, where indicated, the alkyl, alkenyl or alkynyl groups can also be optionally substituted by, for example, oxo, —NR_{d}Rₑ, —CONR_{d}Rₑ, ═NO—Rₑ, —NR_{d}CORₑ, carboxy, —C(═NR_{d})NRₑR_{f}, azido, cyano, C₁₋₆ alkyloxy, C₂₋₆ alkenyloxy, C₂₋₆ alkynyloxy, —N(R_{d})C(═NRₑ)—NR_{f}R_{g}, hydroxyl, nitro, nitroso, —N(Rₕ)CONRᵢRⱼ, —S(O)₀₋₂Rₐ, —C(O)Rₐ, —C(O)ORₐ—, —SO₂NRₐR_{b}, —NRₐSO₂R_{b}, —NRₐSO₂NR_{b}R_{c}, —CRₐN═OR_{b}, and/or —NRₐCOOR_{b}, wherein Rₐ-Rⱼ are each independently H, C₁₋₄ alkyl, C₂₋₄ alkenyl, or C₂₋₄ alkynyl. The “alkyl,” “alkenyl,” and “alkynyl” can also be optionally substituted by —OCONRₑR_{f}.

The terms “cycloalkyl”, and “cycloalkenyl” represent a cyclic hydrocarbon alkyl or alkenyl, respectively, and are meant to include monocyclic (e.g., cyclohexyl), spiro (e.g., spiro[2.3]hexanyl), fused (e.g., bicyclo[4.4.0]decanyl), and bridged (e.g., bicyclo[2.2.1]heptanyl)hydrocarbon moieties. Where indicated, the “cycloalkyl”, and “cycloalkenyl” groups can also be optionally substituted as defined in “alkyl” and “alkenyl” definition.

The terms “alkoxy,” “alkenyloxy,” and “alkynyloxy” represent an alkyl, alkenyl or alkynyl moiety, respectively, which is covalently bonded to the adjacent atom through an oxygen atom. Examples include but are not limited to methoxy, ethoxy, propoxy, isopropoxy, butoxy, isobutoxy, sec-butoxy, tert-butoxy, pentyloxy, isopentyloxy, neopentyloxy, tert-pentyloxy, hexyloxy, isohexyloxy, trifluoromethoxy and neohexyloxy.

Like the alkyl, alkenyl and alkynyl groups, where indicated the alkoxy (—O-alkyl), alkenyloxy (—O-alkenyl), and alkynyloxy (—O-alkynyl) groups can also be optionally substituted. The alkoxy, alkenyloxy, and alkynyloxy groups can be optionally substituted by, for example, halogens, oxo, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, C₁₋₆ alkyl, C₂₋₆ alkenyl, C₂₋₆ alkynyl, —N(Rₕ)CONRᵢRⱼ, —S(O)₀₋₂Rₐ, —C(O)Rₐ, —C(O)ORₐ, ═NO—Rₑ, —SO₂NRₐR_{b}, —NRₐSO₂R_{b}, —NRₐSO₂NR_{b}R_{c}, —CRₐN═OR_{b}, and/or —NRₐCOOR_{b}, wherein Rₐ-Rⱼ are each independently H, C₁₋₄ alkyl, C₂₋₄ alkenyl, or C₂₋₄ alkynyl. The alkoxy (—O-alkyl), alkenyloxy (—O-alkenyl), and alkynyloxy (—O-alkynyl) groups can also be optionally substituted by —OCONRₑR_{f}.

The term “aryl” represents a carbocyclic moiety containing at least one benzenoid-type ring (i.e., may be monocyclic or polycyclic), and which where indicated may be optionally substituted with one or more substituents. Examples include but are not limited to phenyl, tolyl, dimethylphenyl, aminophenyl, anilinyl, naphthyl, anthryl, phenanthryl or biphenyl. The aryl groups can be optionally substituted by, for example, halogens, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₁₋₆ alkyloxy, C₂₋₆ alkenyloxy, C₂₋₆ alkynyloxy, —S(O)₀₋₂Rₐ, optionally substituted 5-12 member heteroaryl, optionally substituted 6-18 member heteroaralkyl, optionally substituted 3-12 member heterocycle, optionally substituted 4-18 member heterocycle-alkyl, —C(O)Rₐ, —C(O)ORₐ, —SO₂NRₐR_{b}, —NRₐSO₂R_{b}, —NRₐSO₂NR_{b}R_{c}, —CRₐN═OR_{b}, and/or —NRₐCOOR_{b}, wherein Rₐ-Rⱼ are each independently H, C₁₋₄ alkyl, C₂₋₄ alkenyl, or C₂₋₄ alkynyl. The aryl group can also be optionally substituted by —OCONRₑR_{f}.

The terms “aryloxy,” represent an aryl moiety substituted with an oxygen, wherein the point of attachement to the molecule it substitutes is on the oxygen. Where indicated the aryloxy group (—O-aryl) can also be optionally substituted by one or more substituents, for example, halogens, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, —N(Rₕ)CONRᵢRⱼ, C₁₋₆ alkyl, C₂₋₆ alkenyl, C₂₋₆ alkynyl, C₁₋₆ alkyloxy, C₂₋₆ alkenyloxy, C₂₋₆ alkynyloxy, S(O)₀₋₂Rₐ, optionally substituted 5-12 member heteroaryl, optionally substituted 6-18 member heteroaralkyl, optionally substituted 3-12 member heterocycle, optionally substituted 4-18 member heterocycle-alkyl, C(O)Rₐ, C(O)ORₐ, SO₂NRₐR_{b}, NRₐSO₂R_{b}, NRₐSO₂NR_{b}R_{c}, CRₐN═OR_{b}, —OCONRₑR_{f} or —NRₐCOOR_{b}, wherein Rₐ-Rⱼ, are each independently H, C₁₋₄ alkyl, C₂₋₄ alkenyl, or C₂₋₄ alkynyl.

The term “aralkyl” represents an aryl group attached to the adjacent atom by an alkyl, alkenyl, or alkynyl. Examples include but are not limited to benzyl, benzhydryl, trityl, phenethyl, 3-phenylpropyl, 2-phenylpropyl, 4-phenylbutyl and naphthylmethyl.

Like the aryl groups, where indicated the aralkyl groups can also be optionally substituted. Where indicated, the aralkyl groups can be optionally substituted by, for example, halogens, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₁₋₆ alkyloxy, C₂₋₆ alkenyloxy, C₂₋₆ alkynyloxy, S(O)₀₋₂Rₐ, optionally substituted 5-12 member heteroaryl, optionally substituted 6-18 member heteroaralkyl, optionally substituted 3-12 member heterocycle, optionally substituted 4-18 member heterocycle-alkyl, —C(O)Rₐ, —C(O)ORₐ, —SO₂NRₐR_{b}, —NRₐSO₂R_{b}, —NRₐSO₂NR_{b}R_{c}, —CRₐN═OR_{b}, and/or —NRₐCOOR_{b}, wherein Rₐ-Rⱼ are each independently H, C₁₋₄ alkyl, C₂₋₄ alkenyl, or C₂₋₄ alkynyl. The aralkyl groups can also be optionally substituted by —OCONRₑR_{f}.

The term “heterocycle” represents an optionally substituted, non aromatic, saturated or partially saturated wherein said cyclic moiety is interrupted by at least one heteroatom selected from oxygen (O), sulfur (S) or nitrogen (N). Heterocycles may be monocyclic or polycyclic rings. For example, a 3-12 member heterocycle is an optionally substituted, non aromatic, saturated or partially saturated cyclic moiety having 3-12 ring atoms wherein at least one ring atom is a heteroatom selected from oxygen (O), sulfur (S) or nitrogen (N). Examples include but are not limited to azetidinyl, dioxolanyl, morpholinyl, morpholino, oxetanyl, piperazinyl, piperidyl, piperidino, cyclopentapyrazolyl, cyclopentaoxazinyl, cyclopentafuranyl, tetrahydrofuranyl, tetrahydrothiofuranyl, tetrahydrothiofuranyl, tetrahydropyranyl, tetrahydrothiopyranyl, tetrahydrothiopyranyl dioxyde, thiazolinyl, oxazolinyl, pyranyl, thiopyranyl, aziridinyl, azepinyl, dioxazepinyl, diazepinyl, oxyranyl, oxazinyl, pyrrolidinyl, thiopyranyl, thiolane, pyrazolidinyl, dioxanyl, and imidazolidinyl. Where indicated, the heterocyclic groups can be optionally substituted by, for example, halogens, oxo, —NR_{d}Rₑ, —CONR_{d}Rₑ, ═NO—Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₇₋₁₂ aralkyl, C₆₋₁₂ aryl, C₁₋₆ alkyloxy, C₂₋₆ alkenyloxy, C₂₋₆ alkynyloxy, —S(O)₀₋₂Rₐ, C₆₋₁₀ aryl, C₆₋₁₀ aryloxy, C₇₋₁₀ arylalkyl, C₆₋₁₀ aryl-C₁₋₁₀ alkyloxy, —C(O)Rₐ, —C(O)ORₐ, —SO₂NRₐR_{b}, —NRₐSO₂R_{b}, —NRₐSO₂NR_{b}R_{c}, —CRₐN═OR_{b}, and/or —NRₐCOOR_{b}, wherein Rₐ-Rⱼ are each independently H, C₁₋₄ alkyl, C₂₋₄ alkenyl or C₂₋₄ alkynyl. The heterocyclic groups can also be optionally substituted by —OCONRₑR_{f}.

The term “heterocycle-alkyl” represents an optionally substituted heterocycle group attached to the adjacent atom by an alkyl, alkenyl, or alkynyl group. It is understood that in a 5-18 member heterocycle-alkyl moiety, the term “5-18 member” represents the total number of ring atoms present in the heterocycle moiety and carbon atoms present in the alkyl, alkenyl or alkynyl portion. For example, the following groups are encompassed by a 7 member heterocycle-alkyl (* represents the attachment point):

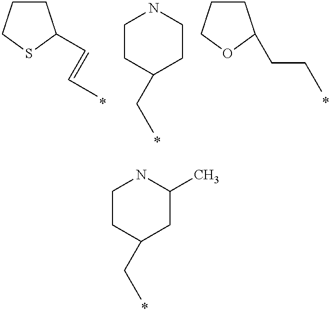

Where indicated the heterocycle-alkyl groups can be optionally substituted by, for example, halogens, oxo, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₁₋₆ alkyloxy, C₂₋₆ alkenyloxy, C₂₋₆ alkynyloxy, —S(O)₀₋₂Rₐ, C₆₋₁₀ aryl, C₆₋₁₀ aryloxy, C₇₋₁₀ arylalkyl, C₆₋₁₀-aryl-C₁₋₁₀ alkyloxy, —C(O)Rₐ, —C(O)ORₐ, ═NO—Rₑ, —SO₂NRₐR_{b}, —NRₐSO₂R_{b}, —NRₐSO₂NR_{b}R_{c}, —CRₐN═OR_{b}, and/or —NRₐCOOR_{b}, wherein Rₐ-Rⱼ are each independently H, C₁₋₄ alkyl, C₂₋₄ alkenyl or C₂₋₄ alkynyl. The heterocycle-alkyl groups can also be optionally substituted by —OCONRₑR_{f}.

The term “heteroaryl” represents an optionally substituted aromatic cyclic moiety wherein said cyclic moiety is interrupted by at least one heteroatom selected from oxygen (O), sulfur (S) or nitrogen (N). Heteroaryls may be monocyclic or polycyclic rings. For example, a 5-12 member heteroaryl is an optionally substituted, aromatic cyclic moiety having 5-12 ring atoms wherein at least one ring atom is a heteroatom selected from oxygen (O), sulfur (S) or nitrogen (N). Examples include but are not limited to—dithiadiazinyl, furanyl, isooxazolyl, isothiazolyl, imidazolyl, oxadiazolyl, dioxazole, oxatriazole, oxazolyl, pyrazinyl, pyridazinyl, pyrimidinyl, pyridyl, pyrazolyl, pyrrolyl, thiatriazolyl, tetrazolyl, thiadiazolyl, triazolyl, thiazolyl, thienyl, tetrazinyl, thiadiazinyl, triazinyl, thiazinyl, furoisoxazolyl, imidazothiazolyl, thienoisothiazolyl, thienothiazolyl, imidazopyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienopyridyl, benzothienyl, benzoxazinyl, benzothiazinyl, quinazolinyl, naphthyridinyl, quinolinyl, isoquinolinyl, benzopyranyl, pyridopyridazinyl and pyridopyrimidinyl. Where indicated the heteroaryl groups can be optionally substituted by, for example, halogens, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₁₋₆ alkyloxy, C₂₋₆ alkenyloxy, C₂₋₆ alkynyloxy, —S(O)₀₋₂Rₐ, C₆₋₁₀ aryl, C₆₋₁₀ aryloxy, C₇₋₁₀ arylalkyl, C₆₋₁₀ aryl-C₁₋₁₀ alkyloxy, —C(O)Rₐ, —C(O)ORₐ, —SO₂NRₐR_{b}, —NRₐSO₂R_{b}, N—RₐSO₂NR_{b}R_{c}, —CRₐN═OR_{b}, and/or —NRₐCOOR_{b}, wherein Rₐ-Rⱼ are each independently H, C₁₋₄ alkyl, C₂₋₄ alkenyl or C₂₋₄ alkynyl. The heteroaryl groups can also be optionally substituted by —OCONRₑR_{f}.

The term “heteroaralkyl” represents an optionally substituted heteroaryl group attached to the adjacent atom by an alkyl, alkenyl, or alkynyl group. Where indicated the heteroaralkyl groups can be optionally substituted by, for example, halogens, —NR_{d}Rₑ, —CONR_{d}Rₑ, —NR_{d}CORₑ, carboxy, —C(═NR_{d})NRₑR_{f}, azido, cyano, —N(R_{d})C(═NRₑ)NR_{f}R_{g}, hydroxyl, nitro, nitroso, —N(Rₕ)CONRᵢRⱼ, C₁₋₆ alkyl, C₂₋₆ alkenyl, C₂₋₆ alkynyl, C₁₋₆ alkyloxy, C₂₋₆ alkenyloxy, C₂₋₆ alkynyloxy, —S(O)₀₋₂Rₐ, C₆₋₁₀ aryl, C₆₋₁₀ aryloxy, C₇₋₁₀ arylalkyl, C₆₋₁₀ aryl-C₁₋₁₀ alkyloxy, —C(O)Rₐ, —C(O)ORₐ, —SO₂NRₐR_{b}, —NRₐSO₂R_{b}, —NRₐSO₂NR_{b}R_{c}, —CRₐN═OR_{b}, and/or —NRₐCOOR_{b}, wherein Rₐ-Rⱼ are each independently H, C₁₋₄ alkyl, C₂₋₄ alkenyl, or C₂₋₄ alkynyl. It is understood that in a 6-18 member heteroaralkyl moiety, the 6-18 member represents the atoms that are present in both the heterocycle moiety and the alkyl, alkenyl or alkynyl groups. It is understood that in a 6-18 member heteroaryl moiety, the term “6-18 member” represents the total number of ring atoms present in the heteroaryl moiety and carbon atoms present in the alkyl, alkenyl or alkynyl portion. For example, the following groups are encompassed by a 7 member heteroaralkyl (* represents the attachment point):

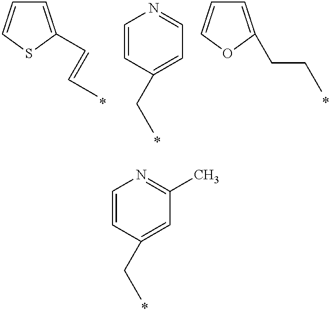

The heteroaralkyl groups can also be optionally substituted by —OCONRₑR_{f}.

“Halogen atom” is specifically a fluorine atom, chlorine atom, bromine atom or iodine atom.

The term “oxo” represents ═O.

A dash (“-”) that is not between two letters or symbols is used to indicate a point of attachement for a substitutent. For example, —CONR_{d}Rₑ is attached through the carbon of the amide.

A bond represented by a combination of a solid and dashed line, ie. [symbol] , may be either a single or double bond.

The term “amidino” represents —C(═NR_{d})NRₑR_{f} wherein R_{d}, Rₑ and R_{f} are each independently selected from H, C₁₋₁₀ alkyl, C₂₋₁₀ alkenyl, C₂₋₁₀ alkynyl, C₆₋₁₂ aryl, and C₇₋₁₂ aralkyl, or Rₑ and R_{f} are taken together with the nitrogen to which they are attached to form an optionally substituted 4 to 10 member heterocycle or an optionally substituted 5-12 member heteroaryl.

The term “guanidino” represents —N(R_{d})C(═NRₑ)NR_{f}R_{g} wherein R_{d}, Rₑ, R_{f} and R_{g} are each independently selected from H, C₁₋₁₀ alkyl, C₂₋₁₀ alkenyl, C₂₋₁₀ alkynyl, C₆₋₁₂ aryl, and C₇₋₁₂ aralkyl, or R_{f} and R_{g} are taken together with the nitrogen to which they are attached to form an optionally substituted 4 to 10 member heterocycle or an optionally substituted 5-12 member heteroaryl.

When there is a sulfur atom present, the sulfur atom can be at different oxidation levels, i.e., S, SO, or SO₂. All such oxidation levels are within the scope of the present invention.

The term “independently” means that a substituent can be the same or a different definition for each item.

The term “hydroxyl protecting group” is well known in the field of organic chemistry. Such protecting groups may be found in “Protective Groups in Organic Synthesis” second edition, Wiley-interscience publication, by T. W. Greene and P. G. M. Wuts. Examples of hydroxy protecting groups include but are not limited to benzyl, acetyl, benzoyl, pivaloyl and isopropyloxycarbonyl.

In still another aspect, there is provided a method for prevention or treatment of HIV infection in a subject in need of such treatment comprising administering to the subject a therapeutically effective amount of a compound of formula (I) or composition of the invention.

In still another aspect, there is provided a method for delaying the onset of AIDS or treating AIDS in a subject in need of such treatment comprising administering to the subject a therapeutically effective amount of a compound of formula (I) or composition of the invention.

In another embodiment, there is provided the use of a compound, composition or combination of the invention for the manufacture of a medicament for treating or preventing HIV infection in a subject in need of such treatment.

In another embodiment, there is provided the use of a compound, composition or combination of the invention for the manufacture of a medicament for blocking cellular entry of HIV in a subject.

In another embodiment, there is provided the use of a compound, composition or combination of the invention for the manufacture of a medicament for delaying the onset of AIDS or treating AIDS in a subject in need of such treatment.

In still another aspect, there is provided a method for blocking cellular entry of HIV in a subject or for the prevention or treatment of HIV infection in a subject in need of such treatment comprising administering to the subject a pharmaceutical combination comprising at least one compound of formula (I) and at least one further therapeutic agent.

In still another aspect, there is provided a method for delaying the onset of AIDS or treating AIDS in a subject in need of such treatment comprising administering to the subject a pharmaceutical combination comprising at least one compound of formula (I) and at least one further therapeutic agent.

In another embodiment, the pharmaceutical combination (e.g., a pharmaceutical composition) of this invention may contain at least one further therapeutic agent which is an antiviral agent.

In one embodiment, the pharmaceutical combination of this invention may contain at least one further antiviral agent which is chosen from nucleoside and nucleotide analog reverse transcriptase inhibitors, non-nucleoside reverse transcriptase inhibitors, protease inhibitors, attachment and fusion inhibitors, integrase inhibitors, and maturation inhibitors.

In one embodiment, the pharmaceutical combinations of this invention may contain at least one other antiviral agent which is a nucleoside and nucleotide analog reverse transcriptase inhibitors chosen from Atripla™ (tenofovir, efavienz, emtricitabine), 3TC (lamivudine, Epivir®), AZT (zidovudine, Retrovir®), Emtricitabine (Coviracil®, formerly FTC), d4T (2′,3′-dideoxy-2′,3′-didehydro-thymidine, stavudine and Zerit®), tenofovir (Viread®), 2′,3′-dideoxyinosine (ddI, didanosine, Videx®), 2′,3′-dideoxycytidine (ddC, zalcitabine, Hivid®), Combivir® (AZT/3TC or zidovudine/lamivudine combination), Trivizir® (AZT/3TC/abacavir or zidovudine/lamivudine/abacavir combination), abacavir (1592U89, Ziagen®), Epzicom® (abacavir and lamivudine), Truvada® (Tenofovir and emtricitabine), SPD-754 (apricitabine), Elvucitabine ACH-126,443 (Beta-L-Fd4C), Alovudine (MIV-310), DAPD (amdoxovir), Racivir, phosphazid, stampidine, CMX-157, PPI-801/802 (formerly MIV-410), MIV-210, fozivudine tidoxil, KP-1461, Fosalvudine (HDP 99.0003), 9-[(2-hydroxymethyl)-1,3-dioxolan-4-yl]guanine, and 2-amino-9-[(2-hydroxymethyl)-1,3-dioxolan-4-yl]adenine.

In another embodiment, the pharmaceutical combination of this invention may contain at least one other antiviral agent which is a non-nucleoside reverse transcriptase inhibitor chosen from Nevirapine (Viramune®, NVP, BI-RG-587), delavirdine (Rescriptor®, DLV), efavirenz (DMP 266, Sustiva®), (+)-Calanolide A, Capravirine (AG1549, formerly S-1153), DPC083, MIV-150, TMC120, Intelence (Etravirine®, TMC125), TMC-278 or BHAP (delavirdine), calanolides, GW695634, RDEA806, RDEA427, RDEA640, UK-453061, BILR355, VRX 840773 and L-697,661 (2-Pyridinone 3-benzoxazolMeNH derivative).

In another embodiment, the pharmaceutical combination of this invention may contain at least one other antiviral agent which is a protease inhibitor chosen from nelfinavir (Viracept®, NFV), amprenavir (141W94, Agenerase®), indinavir (MK-639, IDV, Crixivan®), saquinavir (Invirase®, Fortovase®, SQV), ritonavir (Norvir®, RTV), lopinavir (ABT-378, Kaletra®), Atazanavir (Reyataz®, BMS232632), mozenavir (DMP-450), fosamprenavir (GW433908), RO033-4649, Tipranavir (Aptivus®, PNU-140690), Darunavir (Prezista®, TMC114), SPI-256, Brecanavir (GW640385), P-1946, MK-8122 (formerly PPL-100) and VX-385.

In another embodiment, the pharmaceutical combination of this invention may contain at least one other antiviral agent which is an attachment and fusion inhibitor chosen from T-20 (enfuvirtide, Fuzeon®), T-1249, TRI-999, TRI-1144, Schering C (SCH-C), Vicriviroc (Schering D, SCH-D), FP21399, PRO-140, PRO 542, PRO 452, TNX-355, Aplaviroc (GW873140, AK602), TBR-220 (formerly TAK-220), TBR-652 (formerly TAK-652), PF-232798, Maraviroc (Selzentry®, UK-427,857) or soluble CD4, CD4 fragments, CD4-hybrid molecules, BMS-806, BMS-488043, AMD3100, AMD070, AMD887, INCB9471, INCB15050, KRH-2731, KRH-3140, SJ-3366, SP-01A, sifuvirtide and KRH-3955.

In another embodiment, the pharmaceutical combination of this invention may contain at least one other antiviral agent which is an integrase inhibitor chosen from S-1360, L-870,810, elvitegravir (GS9137, JKT 303), GS9137, L-870,812, raltegravir (Isentress®, MK-0518), MK-2048, GSK1349572, and C-2507.

In another embodiment, the pharmaceutical combination of this invention may contain at least one other antiviral agent which is a maturation inhibitor chosen from Vivecon (MPC-9055) and Bevirimat PA-457.

In another embodiment, the pharmaceutical combination of this invention may contain at least one other antiviral agent which is a zinc finger inhibitor and is azodicarbonamide (ADA).

In another embodiment, the pharmaceutical combination of this invention may contain at least one other antiviral agent which is an antisense drug and is HGTV43.

In another embodiment, the pharmaceutical combination of this invention may contain at least one other antiviral agent which is an immunomodulator, immune stimulator or cytokine chosen from interleukin-2 (IL-2, Aldesleukin, Proleukin), granulocyte macrophage colony stimulating factor (GM-CSF), erythropoietin, Multikine, Ampligen, thymomodulin, thymopentin, foscarnet, HE2000, Reticulose, Murabutide, Resveratrol, HRG214, HIV-1 Immunogen (Remune), WF10 and EP HIV-1090.

In another embodiment, the pharmaceutical combination of this invention may contain at least one other antiviral agent chosen from: 2′,3′-dideoxyadenosine, 3′-deoxythymidine, 2′,3′-dideoxy-2′,3′-didehydrocytidine and ribavirin; acyclic nucleosides such as acyclovir, and ganciclovir; interferons such as alpha-, beta-and gamma-interferon; glucuronation inhibitors such as probenecid; and TIBO drugs, HEPT, Pictovir® (VGX-410) and TSAO derivatives.

In another embodiment, the pharmaceutical combination of this invention may contain an inhibitor of the cytochrome P450.

In another embodiment, the pharmaceutical combination of this invention may contain an inhibitor of the cytochrome P450 chosen from atazanavir, clarithromycin, indinavir, itraconazole, ketoconazole, nefazodone, nelfinavir, ritonavir, saquinavir, telithromycin, amprenavir, erythromycin, fluconazole, fosamprenavir, grapefruit juice, fluvoxamine, fluoxetine, macrolide antibiotics, sertraline sulfaphenazole, Troleandomycin, cyclosporine, clomethiazole, atazanavir, mibefradil, vitamin E, bergamottin, dihydroxybergamottin or pharmaceutically acceptable salts thereof.

In another embodiment, the pharmaceutical combination of this invention may contain an inhibitor of the cytochrome P450 which is ritonavir or pharmaceutically acceptable salts thereof.

The combinations referred to above may conveniently be presented for use in the form of a pharmaceutical formulation and thus pharmaceutical formulations comprising a combination as defined above together with a pharmaceutically acceptable carrier thereof comprises a further aspect of the invention.

The individual components of such combinations may be administered either sequentially or simultaneously in separate or combined pharmaceutical formulations.

In a further embodiment, the compound of formula (I) and at least one further therapeutic agent are administered sequentially.

In a further embodiment, the compound of formula (I) and at least one further therapeutic agent are administered simultaneously.

Thus, a further embodiment of the invention is a kit for use in administering a combination, the kit comprising: a first containment means for storing a compound according to formula I in the form of a pharmaceutical formulation further comprising a pharmaceutically acceptable carrier; and a second containment means for storing at least one further therapeutic agent in the form of a pharmaceutical formulation further comprising a pharmaceutically acceptable carrier.

In one embodiment, the present invention further provides a pharmaceutical composition comprising at least one compound having the formula (I) or a pharmaceutically acceptable salt thereof, or a pharmaceutically acceptable hydrate thereof, or a pharmaceutically acceptable solvate thereof, and at least one pharmaceutically acceptable carrier or excipient.

The terms “host” or “patient” or “subject” means a human, male or female, for example, a child, an adolescent, or an adult.

It will be appreciated that the amount of a compound of the invention required for use in treatment will vary not only with the particular compound selected but also with the route of administration, the nature of the condition for which treatment is required and the age and condition of the patient and will be ultimately at the discretion of the attendant physician or veterinarian. In general, however, a suitable dose will be in the range of from about 0.1 to about 750 mg/kg of body weight per day, for example, in the range of 0.5 to 60 mg/kg/day, or, for example, in the range of 1 to 20 mg/kg/day.

The desired dose may conveniently be presented in a single dose or as divided dose administered at appropriate intervals, for example as two, three, four or more doses per day.

The compound is conveniently administered in unit dosage form; for example containing 10 to 1500 mg, conveniently 20 to 1000 mg, most conveniently 50 to 700 mg of active ingredient per unit dosage form.

Ideally the active ingredient should be administered to achieve peak plasma concentrations of the active compound of from about 1 to about 75 μM, about 2 to 50 μM, about 3 to about 30 μM. This may be achieved, for example, by the intravenous injection of a 0.1 to 5% solution of the active ingredient, optionally in saline, or orally administered as a bolus containing about 1 to about 500 mg of the active ingredient. Desirable blood levels may be maintained by a continuous infusion to provide about 0.01 to about 5.0 mg/kg/hour or by intermittent infusions containing about 0.4 to about 15 mg/kg of the active ingredient.

When the compounds of the present invention or a pharmaceutically acceptable salt thereof is used in combination with a second therapeutic agent active against the same virus the dose of each compound may be either the same as or differ from that when the compound is used alone. Appropriate doses will be readily appreciated by those skilled in the art.

While it is possible that, for use in therapy, a compound of the invention may be administered as the raw chemical it is preferable to present the active ingredient as a pharmaceutical composition. The invention thus further provides a pharmaceutical composition comprising compounds of the present invention or a pharmaceutically acceptable salts thereof together with one or more pharmaceutically acceptable carriers therefore and, optionally, other therapeutic and/or prophylactic ingredients. The carrier(s) must be “acceptable” in the sense of being compatible with the other ingredients of the formulation and not deleterious to the recipient thereof.

Pharmaceutical compositions include those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds according to the invention may also be formulated for parenteral administration (e.g. by injection, for example, bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

For topical administration to the epidermis, the compounds according to the invention may be formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Compositions suitable for topical administration in the mouth include lozenges comprising active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical compositions suitable for rectal administration wherein the carrier is a solid are, for example, presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

Compositions suitable for vaginal administration may be presented as pessaries, tampons, creams, gels, pastes, foams or sprays containing in addition to the active ingredient such carriers as are known in the art to be appropriate.

For intra-nasal administration the compounds of the invention may be used as a liquid spray or dispersible powder or in the form of drops. Drops may be formulated with an aqueous or non-aqueous base also comprising one more dispersing agents, solubilizing agents, or suspending agents. Liquid sprays are conveniently delivered from pressurized packs.

For administration by inhalation the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the compounds according to the invention may take the form of a dry powder composition, for example a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

When desired the above described formulations adapted to give sustained release of the active ingredient may be employed.

Compounds according to the present invention include:

| Cpd# | Name |
|---|---|
| 4 | 17β-tert-Butyloxycarbonylamino-3β-hydroxy-28-norlup-20(29)-ene; |
| 7 | 17β-Amino-3β-hydroxy-28-norlup-20(29)-ene; |
| 8-1 | 3β-O-(3′,3′-Dimethylsuccinyl)-17β-tert-butyloxycarbonylamino-28-norlup- |
| | 20(29)-ene; |
| 8-2 | 3β-O-(2′,2′-Dimethylsuccinyl)-17β-tert-butyloxycarbonylamino-28-norlup- |
| | 20(29)-ene; |
| 8-3 | 3β-O-[(1′S,3′R)-2′,2′,3′-Trimethyl-cyclopentane-3′-carboxylic acid-1′- |
| | carboxyl]-17β-tert-butyloxycarbonylamino-28-norlup-20(29)-ene; |
| 8-4 | 3β-O-(cis-Cyclohexane-3′-carboxylic acid-1′-carboxyl)-17β-[N-tert- |
| | butyloxycarbonyl-amino]-28-norlup-20(29)-ene; |
| 9-1 | 17β-Amino-3β-O-(3′,3′-dimethylsuccinyl)-28-norlup-20(29)-ene; |
| 9-2 | 3β-O-[(1′S,3′R)-2′,2′,3′-Trimethyl-cyclopentane-3′-carboxylic acid-1′- |
| | carboxyl]-17β-amino-28-norlup-20(29)-ene; |
| 9-3 | 3β-O-(cis-Cyclohexane-3′-carboxylic acid-1′-carboxyl)-17β-amino-28- |
| | norlup-20(29)-ene; |
| 10-1 | 17β-Methyl-amino-3β-O-(3′,3′-dimethylsuccinyl)-28-norlup-20(29)-ene; |
| 10-2 | 17β-(Cyclopropylmethyl-amino)-3β-O-(3′,3′-dimethylsuccinyl)-28-norlup- |
| | 20(29)-ene; |
| 10-3 | 3β-O-(3′,3′-Dimethylsuccinyl)-17β-dimethylamino-28-norlup-20(29)-ene; |
| 11-1 | 3β-O-(3′,3′-Dimethylsuccinyl)-17β-[N′-(methoxycarbonyl)ureido]-28- |
| | norlup-20(29)-ene; |
| 11-2 | 3β-O-(3′,3′-Dimethylsuccinyl)-17β-[N′-(tert-butyl)ureido]-28-norlup-20(29)- |
| | ene; |
| 11-3 | 3β-O-(3′,3′-Dimethylsuccinyl)-28-norlup-20(29)-ene-17β-yl-N-carbamoyl-L- |
| | valine methyl ester; |
| 11-4 | 3β-O-(3′,3′-Dimethylsuccinyl)-28-norlup-20(29)-ene-17β-yl-N-carbamoyl-L- |
| | valine; |
| 11-5 | 3β-O-(3′,3′-Dimethylglutaryl)-28-norlup-20(29)-ene-17β-yl-N-carbamoyl-L- |
| | valine; |
| 11-6 | 3β-O-(3′,3′-Dimethylsuccinyl)-17β-[N′-(benzyl)ureido]-28-norlup-20(29)- |
| | ene; |
| 11-7 | 3β-O-(3′,3′-Dimethylsuccinyl)-17β-[N′-(methyl)ureido]-28-norlup-20(29)- |
| | ene; |
| 11-8 | 3β-O-(3′,3′-Dimethylsuccinyl)-17β-[(morpholine-4-carbonyl)-amino]-28- |
| | norlup-20(29)-ene; |
| 11-9 | 3β-O-(3′,3′-Dimethylsuccinyl)-17β-[(4-methyl-piperazine-1-carbonyl)- |
| | amino]-28-norlup-20(29)-ene; |
| 11-10 | 3β-O-(3′,3′-Dimethylsuccinyl)-17β-ureido-28-norlup-20(29)-ene; |
| 11-11 | 3β-O-(3′,3′-Dimethylsuccinyl)-17β-[(piperidine-1-carbonyl)-amino]-28- |
| | norlup-20(29)-ene; |
| 11-12 | 3β-O-(3′,3′-Dimethylsuccinyl)-17β-(N′-phenyl-ureido)-28-norlup-20(29)-ene; |
| 11-13 | 3β-O-(3′,3′-Dimethylsuccinyl)-17β-(N′,N′-dimethyl-ureido)-28-norlup- |
| | 20(29)-ene; |
| 11-14 | 3β-O-(3′,3′-Dimethylsuccinyl)-17β-[N′-(1-methyl-piperidin-4-ylmethyl)- |
| | ureido]-28-norlup-20(29)-ene; |
| 11-15 | 3β-O-(3′,3′-Dimethylsuccinyl)-17β-[3-(5-methyl-[1,3,4]oxadiazol-2-yl)- |
| | ureido]-28-norlup-20(29)-ene; |
| 11-16 | 3β-O-(3′,3′-Dimethylsuccinyl)-17β-(N′-isopropyl-ureido)-28-norlup-20(29)- |
| | ene; |
| 11-17 | 3β-O-(3′,3′-Dimethylsuccinyl)-17β-(N′-4-fluorophenyl-ureido)-28-norlup- |
| | 20(29)-ene; |
| 11-18 | 3β-O-(3′,3′-Dimethylsuccinyl)-17β-(N′-4-fluorophenylmethyl-ureido)-28- |
| | norlup-20(29)-ene; |
| 11-19 | 3β-O-(3′,3′-Dimethylsuccinyl)-17β-(N′-thiazol-2-yl-ureido)-28-norlup- |
| | 20(29)-ene; |
| 11-20 | 3β-O-(3′,3′-Dimethylsuccinyl)-17β-(N′-cyclohexylmethyl-ureido)-28-norlup- |
| | 20(29)-ene; |
| 11-21 | 3β-O-(3′,3′-Dimethylsuccinyl)-17β-(N′-tetrahydropyran-4-ylmethyl-ureido)- |
| | 28-norlup-20(29)-ene; |
| 11-22 | 3β-O-(3′,3′-Dimethylsuccinyl)-17β-(N′-cyclohexyl-ureido)-28-norlup-20(29)- |
| | ene; |
| 11-23 | 3β-O-(3′,3′-Dimethylsuccinyl)-17β-(N′-(S)-1-phenyl-ethyl-ureido)-28-norlup- |
| | 20(29)-ene; |
| 11-24 | 3β-O-(3′,3′-Dimethylsuccinyl)-17β-(N′-isobutylureido)-28-norlup-20(29)- |
| | ene; |
| 11-25 | 3β-O-(3′,3′-Dimethylsuccinyl)-17β-(N′-4,4-difluorocyclohexyl-ureido)-28- |
| | norlup-20(29)-ene; |
| 11-26 | 3β-O-(3′,3′-Dimethylsuccinyl)-17β-(N′-pyridin-4-yl-ureido)-28-norlup- |
| | 20(29)-ene; |
| 11-27 | 3β-O-(3′,3′-Dimethylsuccinyl)-17β-(N′-(R)-1-phenyl-ethyl-ureido)-28-norlup- |
| | 20(29)-ene; |
| 11-28 | 3β-O-(3′,3′-Dimethylsuccinyl)-17β-[N′-(1-methyl-1-phenylethyl)-ureido]-28- |
| | norlup-20(29)-ene; |
| 11-29 | 3β-O-(3′,3′-Dimethylsuccinyl)-17β-[(pyrrolidine-1-carbonyl)-amino]-28- |
| | norlup-20(29)-ene; |
| 12-1 | 3β-O-(3′,3′-Dimethylsuccinyl)-17β-methylsulfonylamino-28-norlup-20(29)- |
| | ene; |
| 13-1 | 17β-Acetylamino-3β-O-(3′,3′-dimethylsuccinyl)-28-norlup-20(29)-ene; |
| 13-2 | 3β-O-(3′,3′-Dimethylsuccinyl)-17β-methoxyoxalyl-amino-28-norlup-20(29)- |
| | ene; |
| 13-3 | 17β-Dimethylaminooxalyl-amino-3β-O-(3′,3′-dimethylsuccinyl)-28-norlup- |
| | 20(29)-ene; |
| 13-4 | 3β-O,17β-N-bis(3′,3′-dimethylsuccinyl)-28-norlup-20(29)-ene; |
| 13-5 | 3β-O,17β-N-bis(3′,3′-dimethylglutaryl)-28-norlup-20(29)-ene; |
| 13-6 | 3β-O-(3′,3′-Dimethylsuccinyl)-17β-phenylacetylamino-28-norlup-20(29)- |
| | ene; |
| 13-7 | 17β-Benzoylamino-3β-O-(3′,3′-dimethylsuccinyl)-28-norlup-20(29)-ene; |
| 13-8 | 3β-O-(3′,3′-Dimethylsuccinyl)-17β-(pyridin-4-ylcarbonyl)-amino-28-norlup- |
| | 20(29)-ene; |
| 13-9 | 17β-(Cyclopropanecarbonyl-amino)-3β-O-(3′,3′-dimethylsuccinyl)-28- |
| | norlup-20(29)-ene; |
| 13-10 | 3β-O-(3′,3′-Dimethylsuccinyl)-17β-isobutyrylamino-28-norlup-20(29)-ene; |
| 13-11 | 3β-O-(3′,3′-Dimethylsuccinyl)-17β-(3-pyrrolidin-1-yl-propionylamino)-28- |
| | norlup-20(29)-ene; |
| 13-12 | 3β-O-(3′,3′-Dimethylsuccinyl)-17β-[(5-methyl-[1,3,4]oxadiazole-2- |
| | carbonyl)-amino]-28-norlup-20(29)-ene; |
| 13-13 | 3β-O-(3′,3′-Dimethylsuccinyl)-17β-[(thiazol-4-ylcarbonyl)-amino]-28-norlup- |
| | 20(29)-ene; |
| 13-14 | 3β-O-(3′,3′-Dimethylsuccinyl)-17β-[(1-methyl-1H-pyrazol-4-ylcarbonyl)- |
| | amino]-28-norlup-20(29)-ene; |
| 13-15 | 3β-O-(cis-Cyclohexane-3′-carboxylic acid-1′-carboxyl)-17β-(pyridin-4- |
| | ylcarbonyl)-amino-28-norlup-20(29)-ene; |
| 14-1 | 3β-O-(3′,3′-Dimethylsuccinyl)-17β-methoxycarbonylamino-28-norlup- |
| | 20(29)-ene; |
| 14-2 | 3β-O-(3′,3′-Dimethylsuccinyl)-17β-benzyloxycarbonylamino-28-norlup- |
| | 20(29)-ene; |
| 18-1 | 17β-Amino-3β-O-(3′,3′-dimethylsuccinyl)-28-norlupane; |
| 22-1 | 3β-O-(3′,3′-Dimethylsuccinyl)-17β-acetylamino-28-norlupane; |
| 23-1 | 3β-O-(3′,3′-Dimethylsuccinyl)-17β-methoxycabonylamino-28-norlupane; |

and pharmaceutically acceptable salts thereof.

### EXAMPLES

The following general schemes and examples are provided to illustrate various embodiments of the present invention and shall not be considered as limiting in scope. It will be appreciated by those of skill in the art that other compounds of the present invention can be obtained by substituting the generically or specifically described reactants and/or operating conditions used in the following examples.

In the foregoing and in the following examples, all temperatures are set forth uncorrected in degrees Celsius; and, unless otherwise indicated, all parts and percentages are by weight.

Analytical HPLC is carried out under standard conditions using a Phenomenex Gemini C18 column, 250×4.6 mm, 3 μm, 110 Å for the methods A, B, C, D, E and F and a Varian Pursuit XRs C18 column, 50×4.6 mm, 3 μm, for the methods G, H and I. Elution is performed using a linear gradient with a flow rate of 1 mL/min. as described in the following table (solvent A is 0.01% TFA in water; solvent B is 0.01% TFA in MeCN):

| Methods | Solvent B |
|---|---|
| A | 60 to 100% over 40 min |
| B | 50 to 90% over 40 min |
| C | 20 to 60% over 40 min |
| D | 30 to 70% over 40 min |
| E | 70 to 100% over 40 min |
| F | 40 to 80% over 40 min |
| G | 50 to 95% over 15 min |
| H | 75 to 95% over 20 min |
| I | 30 to 75% over 15 min |

The following abbreviations may be used as follows:
Ac₂O Acetic anhydride
BOC tert-butyl carbamate
BOC₂O Di-tert-butyl dicarbonate
br broad
DABCO 1,4-diazabicyclo[2.2.2]octane
DCM dichloromethane
DIPEA Diisopropylethylamine
DMAP 4-Dimethylaminopyridine
DMF N,N-dimethyl formamide
DPPA Diphenylphosphoryl azide
Et₂O Diethyl ether
Hal halogen
PCC Pyridinium chlorochromate
Sept. Septuplet
TEA Triethylamine
TFA Trifluoroacetic acid
THF Tetrahydrofuran

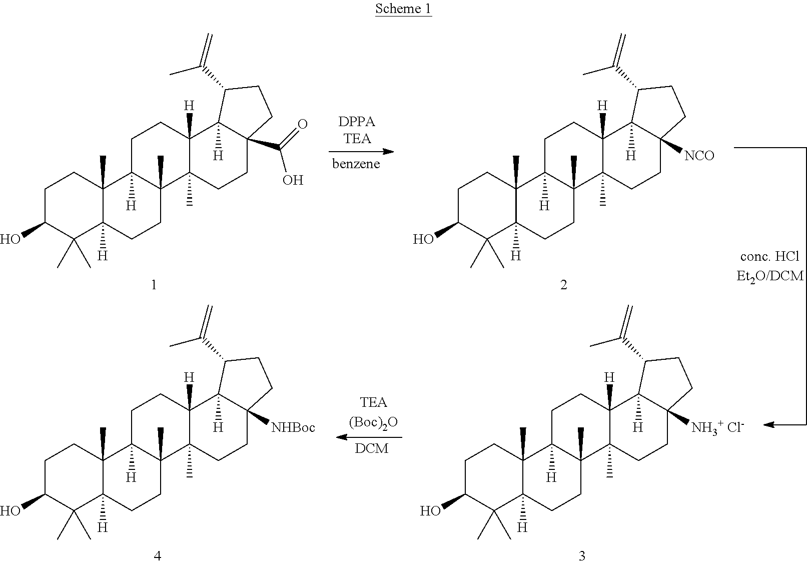

### 3β-Hydroxy-28-norlup-20(29)-ene-17β-isocyanate 2

To a stirring suspension of Betulinic acid 1 (10.15 g, 22.2 mmol) in benzene (180 mL) is added TEA (3.72 mL, 26.7 mmol) and DPPA (7.34 g, 26.7 mmol). The mixture is stirred for 48 hours at room temperature and concentrated to dryness. The residue is purified by flash chromatography on silica gel (ethyl acetate/hexanes 0% to 10%) to afford the title compound 2 (3.6 g, 35%) as a white solid.

IR (v, cm⁻¹): 2259 (NCO) (See ref.: O. B. Flekhter, et al. Russ. J. Bioorg. Chem. 2003, 29, 594-600).

### 17β-Amino-3β-hydroxy-28-norlup-20(29)-ene hydrochloride 3

To a stirring solution of compound 2 (749 mg, 1.75 mmol) in DCM (22 mL) and diethyl ether (18 mL) is added concentrated HCl (5 mL). The biphasic mixture is stirred overnight at room temperature, and then concentrated to dryness. The solid residue is triturated in DCM and collected by filtration to give the title compound 3 (158 mg, 19%). The filtrate is purified by flash chromatography on silica gel (methanol/DCM 0% to 6%) to give the title compound 3 (416 mg, 56%) as a white solid.

¹H NMR (400 MHz, CDCl₃): δ [ppm] 7.55 (s, 3H), 4.71 (d, 1H), 4.62 (d, 1H), 4.28 (br s, 1H), 2.96 (t, 1H), 2.56 (m, 1H), 2.02 (m, 1H), 1.82 (m, 2H), 1.75-0.60 (m, 21H), 1.65 (s, 3H), 1.01 (s, 3H), 0.92 (s, 3H), 0.86 (s, 3H), 0.77 (s, 3H), 0.64 (s, 3H). (See ref.: M. Evers, et al. J. Med. Chem. 1996, 39, 1056-1068).

17β-tert-Butyloxycarbonylamino-3β-hydroxy-28-norlup-20(29)-ene 4

To a stirring solution of compound 3 (535 mg, 1.25 mmol) in DCM (20 mL) is added successively TEA (0.175 mL, 1.25 mmol) and (Boc)₂O (357 mg, 1.64 mmol). The solution is stirred overnight at room temperature then diluted with DCM and washed with 5% citric acid and water, dried over Na₂SO₄ and concentrated to dryness. The residue is purified by flash chromatography on silica gel (ethyl acetate/hexanes 0% to 25%) to afford the title compound 4 as a foam (537 mg, 81%).

¹H NMR (400 MHz, CDCl₃): δ [ppm] 4.70 (d, 1H), 4.59 (m, 1H), 4.32 (s, 1H), 3.18 (d×d, 1H), 2.53 (m, 1H), 2.43 (m, 1H), 2.36 (m, 1H), 1.97 (m, 1H), 1.70-0.60 (m, 21H), 1.67 (s, 3H), 1.43 (s, 9H), 1.0 (s, 3H), 0.96 (s, 3H), 0.95 (s, 3H), 0.82 (s, 3H), 0.75 (s, 3H), 0.67 (d, 1H).

Compound 4 can also be prepared in four steps as described in Scheme 2.

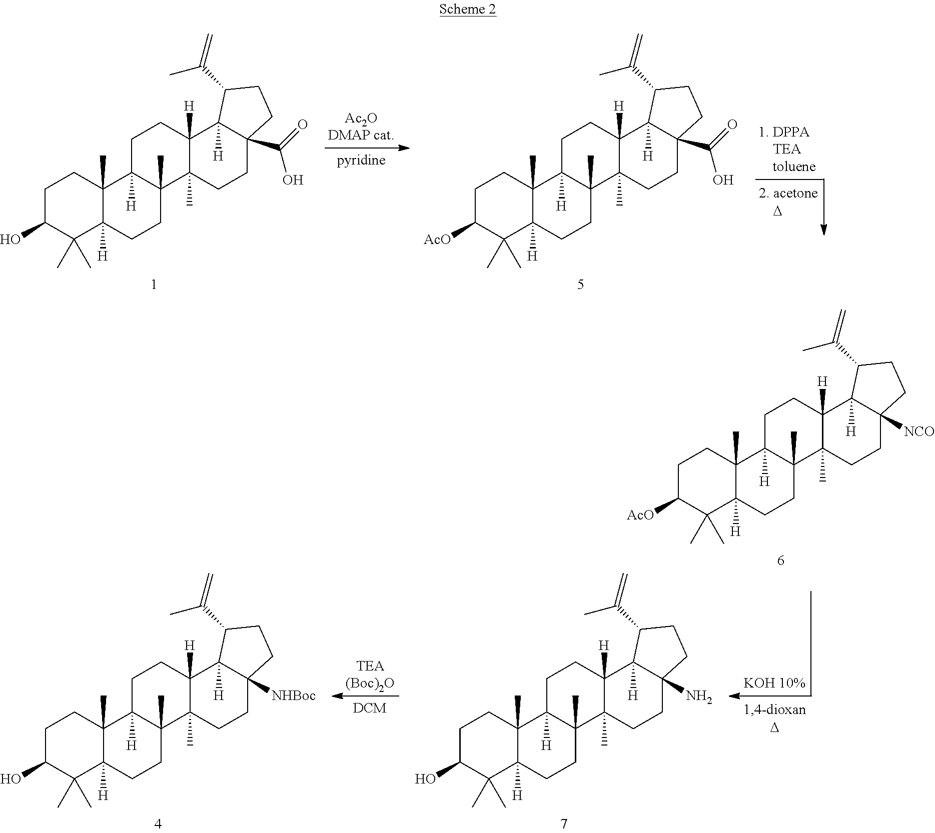

### 3β-O-Acetyl-betulinic acid 5

To a stirring solution of Betulinic acid 1 (25.5 g, 55.8 mmol) in pyridine is added DMAP (682 mg, 5.6 mmol) and acetic anhydride (17 mL, 179.8 mmol). The solution is stirred 1.5 hours at room temperature and then concentrated to dryness. The residue is diluted in ethyl acetate, washed twice with HCl 1N, water and brine, diluted with DCM, dried over sodium sulfate and concentrated to dryness. The solid residue is triturated in ethyl acetate and collected by filtration to give the title compound 5 as a white solid (14.68 g). The filtrate is recovered and purified by flash column chromatography on silica gel (ethyl acetate/hexanes 0% to 15%) to yield more of the title compound 5 as a white solid (5.57 g, 72% total yield).

¹H NMR (400 MHz, CDCl₃): δ [ppm] 4.72 (d, 1H), 4.59 (d×d, 1H), 4.45 (d×d, 1H), 2.99 (t×d, 1H), 2.24 (d×t, 1H), 2.16 (t×d, 1H), 2.02 (s, 3H), 1.97 (m, 2H), 1.70-0.85 (m, 19H), 1.68 (s, 3H), 0.95 (s, 3H), 0.91 (s, 3H), 0.83 (s, 3H), 0.82 (s, 3H), 0.81 (s, 3H), 0.78 (d, 1H).

3β-O-Acetyl-28-norlup-20(29)-ene-17β-isocyanate 6

To a stirring solution of compound 5 (10.12 g, 20.30 mmol) and TEA (3.40 mL, 24.36 mmol) in toluene (100 mL) is added over 1 hour DPPA (4.88 g, 22.33 mmol). The mixture is stirred for 20 hours at room temperature and concentrated to dryness. The residue is purified by flash chromatography on silica gel (100% toluene) to afford a mixture of carbonyl azide and isocyanate as a white solid. This solid is suspended in acetone and refluxed overnight. After cooling, the solid is collected by filtration to give the title compound 6 (9.082 g, 90%) as a white solid.

IR (v, cm⁻¹): 2261 (NCO).

¹H NMR (400 MHz, CDCl₃): δ [ppm] 4.72 (d, 1H), 4.61 (t, 1H), 4.45 (m, 1H), 2.52 (t×d, 1H), 2.09 (m, 1H), 2.03 (s, 3H), 1.87-1.73 (m, 4H), 1.70-0.90 (m, 18H), 1.66 (s, 3H), 1.03 (s, 3H), 0.91 (s, 3H), 0.85 (s, 3H), 0.83 (s, 3H), 0.82 (s, 3H), 0.76 (d, 1H).

17β-Amino-3β-hydroxy-28-norlup-20(29)-ene 7

To a stirring suspension of compound 6 (9.57 g, 19.30 mmol) in 1,4-dioxan (120 mL) is added a solution of potassium hydroxide (7.4 g) in water (70 mL). The mixture is refluxed 6 hours, cooled down to room temperature, diluted with diethyl ether and washed with water. The aqueous layer is back extracted with ether. The combined organic extracts are washed with brine, dried over sodium sulfate and concentrated to dryness to give the title compound 7 as a foam (9.69 g).

¹H NMR (400 MHz, DMSO-d₆): δ [ppm] 4.63 (d, 1H), 4.50 (d×d, 1H), 4.24 (d, 1H), 2.92 (m, 1H), 2.51 (m, 1H), 1.94 (m, 1H), 1.69 (m, 2H), 1.64-0.58 (m, 21H), 1.59 (s, 3H), 0.95 (s, 3H), 0.86 (s, 3H), 0.84 (s, 3H), 0.73 (s, 3H), 0.62 (s, 3H).

17β-tert-Butyloxycarbonylamino-3β-hydroxy-28-norlup-20(29)-ene 4

Compound 4 is prepared from compound 7 using the same condition described in the third reaction of scheme 1.

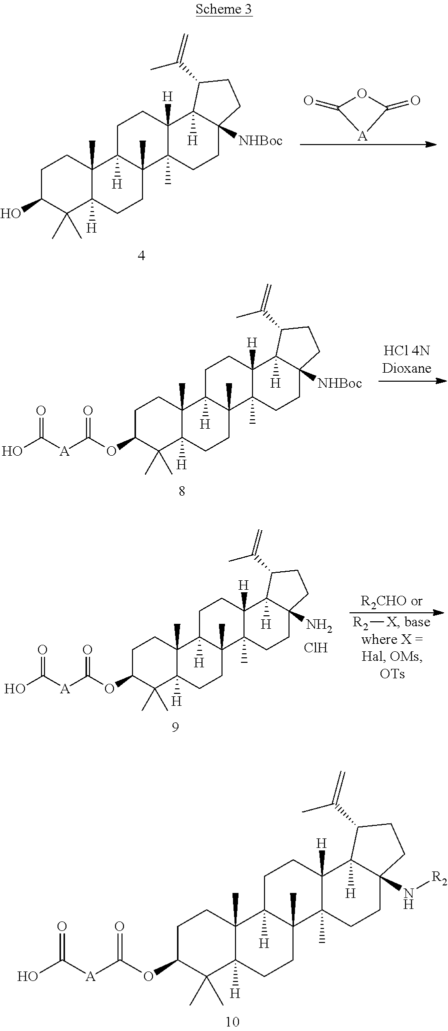

### General Procedure for the Preparation of Compounds 9 or 10:

Step 1: The compound 4 is treated with a base such as DMAP, TEA, DABCO or DIPEA and the appropriate cyclic anhydride (3 to 10 equivalents) in solvents such as pyridine, TEA or toluene (0.2-1.0 M) at temperature between 90 to 130° C. for 4 to 24 hours. The mixture is concentrated, washed with aqueous acid and purified by flash column chromatography on silica gel to yield the compound 8.

Step 2: The protecting group of the compound 8 is then removed with anhydrous HCl to afford the compound 9 as the hydrochloride salt.

Step 3: An alkyl substituent R₂ is introduced by conventional reductive amination with an aldehyde or a ketone (see A. F. Abdel-Magid, et al. J. Org. Chem. (1996), 61, 3849-3862) or by alkylation with an alkyl halide (R₂X) in presence of a base such as TEA, DIPEA or sodium hydride in a solvent such as THF or DMF to give compound 10.

### 3β-O-(3′,3′-Dimethylsuccinyl)-17β-tert-butyloxycarbonylamino-28-norlup-20(29)-ene 8-1

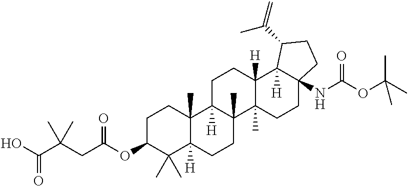

A stirring solution of compound 4 (529 mg, 1 mmol), DMAP (147 mg, 1.2 mmol) and 2,2-dimethylsuccinic anhydride (385 mg, 3 mmol) in dry pyridine (10 mL) is heated for 4 hours at 120° C. Another 3 mmol of 2,2-dimethylsuccinic anhydride is added and heating at 120° C. is continued overnight. The mixture is cooled down to room temperature and concentrated to dryness. The residue is diluted in ethyl acetate, washed twice with HCl 1N, water and brine, dried over sodium sulfate and concentrated to dryness. The residue is purified by flash column chromatography on silica gel (ethyl acetate/hexanes 0% to 30%) to yield the title compound 8-1 as a white solid (631 mg, 94%) and the minor isomer 8-2 (50 mg).

¹H NMR (400 MHz, CDCl₃): δ [ppm] 4.70 (d, 1H), 4.59 (t, 1H), 4.48 (d×d, 1H), 4.35 (br s, 1H), 2.66 (d, 1H), 2.55 (d, 1H), 2.55 (m, 1H), 2.45 (m, 1H), 2.35 (m, 1H), 1.70-0.90 (m, 21H), 1.67 (s, 3H), 1.43 (s, 9H), 1.29 (s, 3H), 1.28 (s, 3H), 1.0 (s, 3H), 0.94 (s, 3H), 0.83 (s, 3H), 0.82 (s, 3H), 0.80 (s, 3H).

LC/MS: m/z=641.74 (M+H⁺).

HPLC (Method A): t_{R}=41.62 min.

### 17β-Amino-3β-O-(3′,3′-dimethylsuccinyl)-28-norlup-20(29)-ene hydrochloride salt 9-1

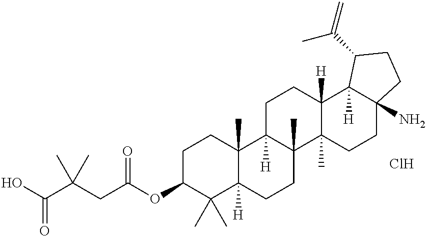

A solution of compound 8-1 (467 mg, 0.712 mmol) in 4 M HCl in 1,4-dioxane is stirred at room temperature overnight. The solvent is evaporated under reduce pressure. The residue is dissolved in ethyl acetate and hexanes is added while stirring to get a white precipitate which is collected by filtration to give the title compound 9-1 (382 mg, 90%) as a white solid.

¹H NMR (400 MHz, DMSO-d₆): δ [ppm] 7.53 (br s, 3H), 4.71 (d, 1H), 4.62 (s, 1H), 4.35 (d×d, 1H), 2.60 (m, 1H), 2.54 (m, 1H), 2.48 (m, 1H), 2.03 (m, 1H), 1.83 (m, 1H), 1.75 (m, 1H), 1.70-0.80 (m, 21H), 1.65 (s, 3H), 1.50 (s, 3H), 1.14 (s, 3H), 1.01 (s, 3H), 0.93 (s, 3H), 0.80 (s, 3H), 0.76 (s, 3H), 0.77 (s, 3H).

LC/MS: m/z=556.61 (M+H⁺).

HPLC (Method A): t_{R}=3.07 min.

### 17β-Methyl-amino-3β-O-(3′,3′-dimethylsuccinyl)-28-norlup-20(29)-ene 10-1

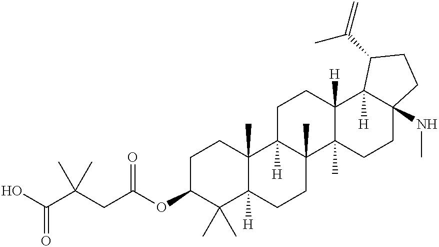

To a solution of compound 9-1 (41 mg, 0.070 mmol) in 1,2-dichloroethane (2 mL) is added TEA (0.0097 mL, 0.070 mmol) followed by a solution of paraformaldehyde (32 mg, 1.05 mmol) in 1,2-dichloroethane (0.3 mL). The reaction is stirred at room temperature for 0.5 hour and then sodium triacetoxyborohydride (18 mg, 0.087 mmol) is added and the reaction stirred overnight at room temperature. The solvent is evaporated under reduced pressure. The residue is purified by flash column chromatography on silica gel (methanol/DCM 0 to 10%) to yield the title compound 10-1 as a white solid (9 mg, 23%).

¹H NMR (400 MHz, CDCl₃): δ [ppm] 4.72 (d, 1H), 4.58 (s, 1H), 4.46 (d×d, 1H), 3.47 (s, 3H), 2.64 (d×d, 2H), 2.56 (m, 1H), 2.05 (m, 1H), 2.0-0.80 (m, 23H), 1.69 (s, 3H), 1.29 (s, 3H), 1.27 (s, 3H), 0.97 (s, 3H), 0.93 (s, 3H), 0.84 (s, 3H), 0.82 (s, 3H), 0.79 (s, 3H). LC/MS: m/z=570.84 (M+H⁺).

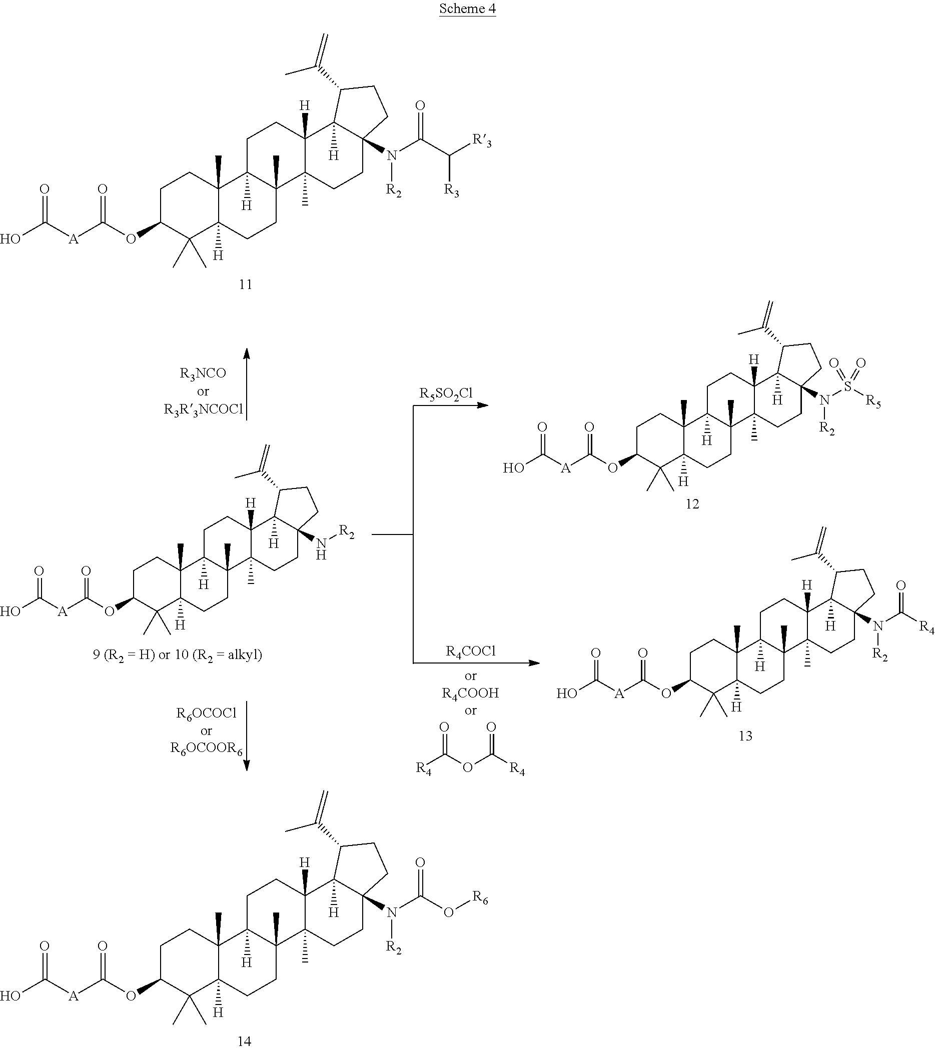

### General Procedures:

Ureas 11 are made by treatment of compound 9 or 10 with an isocyanate, a carbamoyl chloride or phosgene or triphosgene followed by an amine in a solvent such as toluene or THF.

Sulfonamides 12 are obtained by coupling 9 or 10 with the appropriate sulfonyl chloride in solvents such as THF or DCM and in the presence of a base such as TEA or DIPEA.

Amides 13 are prepared by coupling compound 9 or 10 with the appropriate acyl chloride or mixed anhydride or symmetric anhydride or pre-activated carboxylic acid in solvents such as THF or DCM and in the presence of a base such as TEA or DIPEA.

Carbamates 14 are obtained by reacting compound 9 or 10 with the appropriate chloroformate or symmetric carbonate in solvents such as THF or DCM and in the presence of a base such as TEA or DIPEA.

### 3β-O-(3′,3′-Dimethylsuccinyl)-17β-[N′-(methoxycarbonyl)ureido]-28-norlup-20(29)-ene 11-1

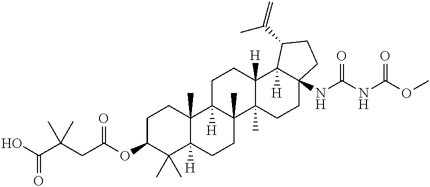

To a stirring solution of compound 9-1 (27 mg, 0.046 mmol) in dry toluene (1.5 mL) is added TEA (0.008 mL, 0.055 mmol) and methyl isocyanatoformate (0.012 mL, 0.130 mmol). The mixture is stirred at room temperature for 3 hours and concentrated to dryness. The residue is purified by flash column chromatography on silica gel (methanol/DCM 0% to 5%) to yield the title compound 11-1 (24 mg, 80%) as a white solid.

¹H NMR (400 MHz, CDCl₃): δ [ppm] 10.28 (s, 1H), 8.13 (s, 1H), 4.73 (s, 1H), 4.60 (s, 1H), 4.46 (d×d, 1H), 3.78 (s, 3H), 2.98 (d, 1H), 2.64 (m, 1H), 2.47 (t×d, 1H), 2.34 (d, 1H), 2.29 (m, 1H), 1.96 (m, 1H), 1.80-0.80 (m, 21H), 1.67 (s, 3H), 1.26 (s, 3H), 1.20 (s, 3H), 1.04 (s, 3H), 0.95 (s, 3H), 0.79 (s, 3H), 0.77 (s, 3H), 0.73 (s, 3H).

LC/MS: m/z=657.67 (M+H⁺).

### 3β-O-(3′,3′-Dimethylsuccinyl)-17β-methylsulfonylamino-28-norlup-20(29)-ene 12-1

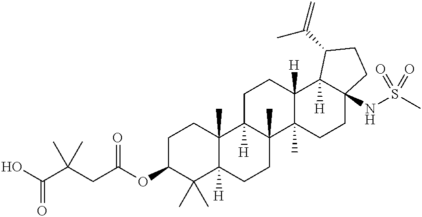

To a stirring solution of compound 9-1 (63 mg, 0.107 mmol) in dry THF (1 mL) is added TEA (0.03 mL, 0.214 mmol) and methanesulfonyl chloride (0.01 mL, 0.128 mmol). The mixture is stirred at room temperature for 2 hours. More methanesulfonyl chloride is added (0.01 mL, 0.128 mmol) and the mixture is stirred overnight at room temperature. The mixture is then diluted with ethyl acetate, washed twice with water and brine, dried over sodium sulfate. The residue is purified by flash column chromatography on silica gel (methanol/DCM 0% to 10%) to yield the title compound 12-1 (4 mg, 6%) as a white solid.

¹H NMR (400 MHz, CDCl₃): δ [ppm] 4.70 (s, 1H), 4.62 (s, 1H), 4.47 (d×d, 1H), 4.05 (s, 1H), 3.01 (s, 3H), 2.66 (d, 1H), 2.55 (d, 1H), 2.48 (m, 1H), 2.42 (m, 1H), 2.35 (m, 1H), 2.04 (m, 1H), 1.81 (m, 1H), 1.70-0.75 (m, 20H), 1.68 (s, 3H), 1.30 (s, 3H), 1.28 (s, 3H), 1.02 (s, 3H), 0.95 (s, 3H), 0.83 (s, 6H), 0.80 (s, 3H).

LC/MS: m/z=539.64 (M+H⁺).

HPLC (Method A): t_{R}=25.07 min.

### 17β-Acetylamino-3β-O-(3′,3′-dimethylsuccinyl)-28-norlup-20(29)-ene 13-1

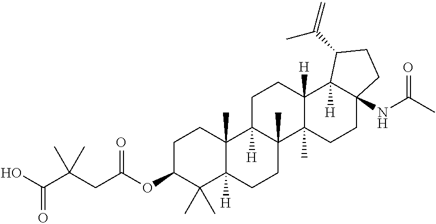

To a stirring solution of compound 9-1 (64 mg, 0.108 mmol) in dry THF (1 mL) is added TEA (0.03 mL, 0.216 mmol) and acetyl chloride (0.01 mL, 0.130 mmol). The mixture is stirred at room temperature for 2 hours, diluted with ethyl acetate, washed twice with water and brine, dried over sodium sulfate. The residue is purified by flash column chromatography on silica gel (methanol/DCM 0% to 3%) to yield the title compound 13-1 (31 mg, 48%) as a white solid.

¹H NMR (400 MHz, CDCl₃): δ [ppm] 5.11 (s, 1H), 4.70 (d, 1H), 4.61 (t, 1H), 4.46 (d×d, 1H), 2.72 (d, 1H), 2.68 (m, 1H), 2.52 (d, 1H), 2.44 (d×d, 1H), 2.38 (m, 1H), 2.04 (s, 3H), 1.95 (m, 1H), 1.67-0.90 (m, 21H), 1.67 (s, 3H), 1.28 (s, 3H), 1.26 (s, 3H), 1.0 (s, 3H), 0.95 (s, 3H), 0.83 (s, 3H), 0.81 (s, 3H), 0.80 (s, 3H).

LC/MS: m/z=597.89 (M+H⁺).

HPLC (Method A): t_{R}=23.88 min.

### 3β-O-(3′,3′-Dimethylsuccinyl)-17β-methoxyoxalyl-amino-28-norlup-20(29)-ene 13-2

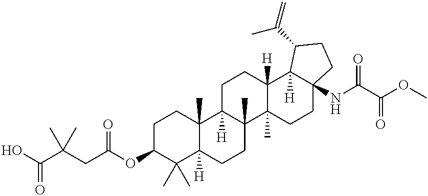

To a stirring solution of compound 9-1 (53 mg, 0.090 mmol) in dry THF (1.5 mL) is added TEA (0.025 mL, 0.180 mmol) and methyl chlorooxoacetate (0.17 mL, 0.180 mmol). The mixture is stirred at room temperature for 2 hours, diluted with ethyl acetate, washed twice with water and brine, dried over sodium sulfate. The residue is purified by flash chromatography on silica gel (methanol/DCM 0% to 4%) to yield the title compound 13-2 (42 mg, 73%) as a foam.

¹H NMR (400 MHz, CDCl₃): δ [ppm] 6.99 (s, 1H), 4.73 (d, 1H), 4.63 (t, 1H), 4.47 (d×d, 1H), 3.90 (s, 3H), 2.66 (d, 1H), 2.60 (m, 1H), 2.54 (d, 1H), 2.48 (m, 1H), 2.45 (m, 1H), 1.87 (m, 1H), 1.72-0.74 (m, 21H), 1.68 (s, 3H), 1.29 (s, 3H), 1.27 (s, 3H), 0.97 (s, 3H), 0.96 (s, 3H), 0.83 (s, 3H), 0.82 (s, 3H), 0.79 (s, 3H).

LC/MS: m/z=642.72 (M+H⁺).

HPLC (Method A): t_{R}=27.67 min.

### 17β-Dimethylaminooxalyl-amino-3β-O-(3′,3′-dimethylsuccinyl)-28-norlup-20(29)-ene 13-3

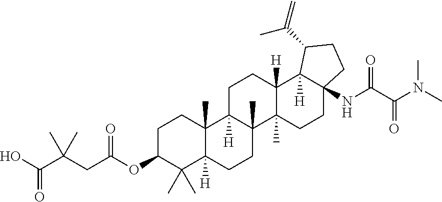

The compound 13-2 (28 mg, 0.044 mmol) is dissolved in a 2.0 M solution of dimethylamine in THF (2.0 mL) and heated for 3 hours at 90° C. in a sealed tube. After concentration, the residue is purified by flash chromatography on silica gel (methanol/DCM 0% to 8%) to yield the title compound 13-3 (18 mg, 64%) as a white solid.

¹H NMR (400 MHz, CDCl₃): δ [ppm] 7.29 (s, 1H), 4.70 (d, 1H), 4.60 (t, 1H), 4.47 (d×d, 1H), 3.41 (s, 3H), 3.02 (s, 3H), 2.65 (d, 1H), 2.60 (m, 1H), 2.54 (d, 1H), 2.48 (m, 1H), 2.44 (m, 1H), 1.89 (m, 1H), 1.70-0.70 (m, 21H), 1.66 (s, 3H), 1.29 (s, 3H), 1.27 (s, 3H), 1.01 (s, 3H), 0.94 (s, 3H), 0.81 (s, 6H), 0.79 (s, 3H).

LC/MS: m/z=655.58 (M+H⁺).

HPLC (Method B): t_{R}=32.69 min.

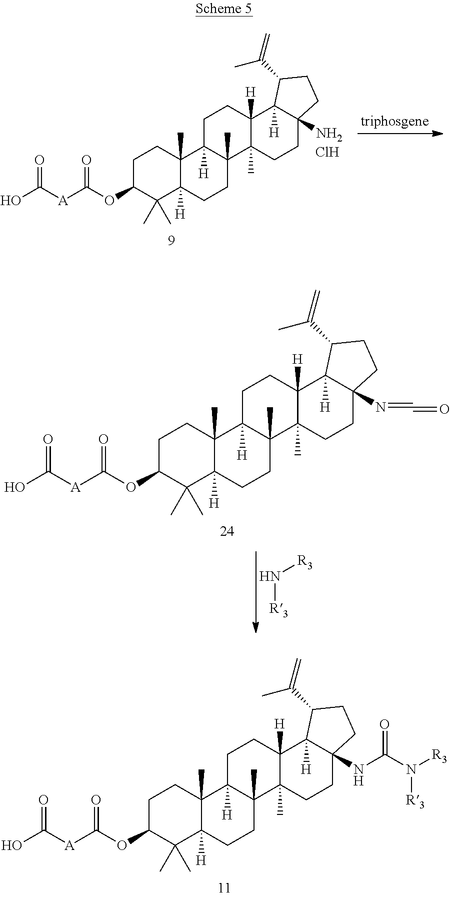

### General Procedure:

Ureas 11 are made by treatment of compound 9 with a phosgene or triphosgene followed by an amine in a solvent such as toluene or THF in the presence of a base such as TEA or DIPEA.

### 3β-O-(3′,3′-Dimethylsuccinyl)-17β-[(morpholine-4-carbonyl)-amino]-28-norlup-20(29)-ene 11-8

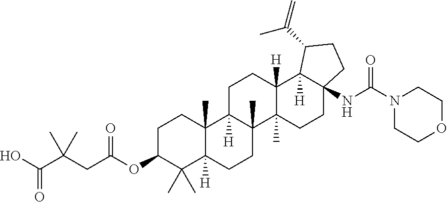

Step 1: To an ice-cold stirring solution of compound 9-1 (353 mg, 0.596 mmol) in dry THF (6 mL) is added DIPEA (0.26 mL, 1.49 mmol) and a solution of triphosgene (354 mg, 1.192 mmol) in THF (3 mL). The mixture is stirred at room temperature for 2.5 hours. HCl 1N (3 mL) is added drop wise, then the mixture is diluted with ethyl acetate, washed with water and brine, dried over sodium sulfate. The residue is purified by flash column chromatography on silica gel (methanol/DCM 0% to 5%) to yield 3β-O-(3′,3′-dimethylsuccinyl)-28-norlup-20(29)-ene-17β-isocyanate (192 mg) as a white solid.

¹H NMR (400 MHz, CDCl₃): δ [ppm] 4.72 (d, 1H), 4.61 (t, 1H), 4.46 (d×d, 1H), 2.66 (d, 1H), 2.54 (d, 1H), 2.52 (m, 1H), 2.09 (m, 1H), 1.86-1.72 (m, 3H), 1.70-0.70 (m, 20H), 1.66 (s, 3H), 1.29 (s, 3H), 1.27 (s, 3H), 1.03 (s, 3H), 0.91 (s, 3H), 0.84 (s, 3H), 0.82 (s, 3H), 0.79 (s, 3H).

IR (v, cm⁻¹): 2260 (NCO)

Step 2: To a stirring solution of 3β-O-(3′,3′-dimethylsuccinyl)-28-norlup-20(29)-ene-17β-isocyanate (56 mg, 0.096 mmol) in toluene (1 mL) is added morpholine (0.042 mL, 0.481 mmol). The mixture is stirred for 1.5 hours at 80° C., cooled down and concentrated to dryness. The residue is purified by flash column chromatography on silica gel (methanol/DCM 0% to 10%) to yield the title compound 11-8 (48 mg) as a white solid.

¹H NMR (400 MHz, CDCl₃): δ [ppm] 4.69 (s, 1H), 4.60 (s, 1H), 4.46 (d×d, 1H), 4.19 (s, 1H), 3.69 (m, 4H), 3.35 (m, 4H), 2.64 (d, 1H), 2.62 (m, 1H), 2.54 (d, 1H), 2.50 (m, 1H), 2.34 (t×d, 1H), 1.95 (m, 1H), 1.70-0.70 (m, 21H), 1.67 (s, 3H), 1.28 (s, 3H), 1.26 (s, 3H), 0.96 (s, 3H), 0.95 (s, 3H), 0.81 (s, 6H), 0.79 (s, 3H).

LC/MS: m/z=667.74 (M+H⁺).

HPLC (Method B) t_{R}=34.077 min.

Table 1 of compounds illustrates some of the compounds of the present invention which are synthesized using the procedures described in schemes 3, 4, and 5.

Retention time (t_{R}) for each compound are measured using the standard analytical HPLC methods described above.

TABLE 1

| | | | t_{R}(min) | |
|---|---|---|---|---|
| Cpd # | Structure | Name | (Method) | M + H⁺ |
| 8-2 | 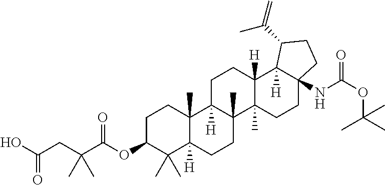 | 3β-O-(2′,2′- Dimethylsuccinyl)- 17β-tert- butyloxycarbonylamino- 28-norlup-20(29)- ene | 41.72 (A) | 641.67 |
| 8-3 | 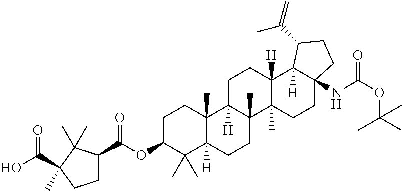 | 3β-O- [(1′S,3′R)- 2′,2′,3′-Trimethyl- cyclopentane-3′- carboxylic acid-1′- carboxyl]-17β-tert- butyloxycarbonylamino- 28-norlup-20(29)- ene | — | 711.09 |
| 8-4 | 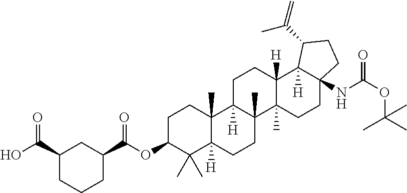 | 3β-O-(cis- Cyclohexane-3′- carboxylic acid-1′- carboxyl)-17β-[N- tert- butyloxycarbonyl- amino]-28-norlup- 20(29)-ene | ¹H NMR (400 MHz, CDCl₃): δ [ppm] 4.70 (s, 1H), 4.60 (s, 1H), 4.44 (m, 1H), 4.37 (s, 1H), 2.70-2.18 (m, 6H), 1.98 (m, 4H), 1.80-0.72 (m, 51H). | |
| 9-2 | 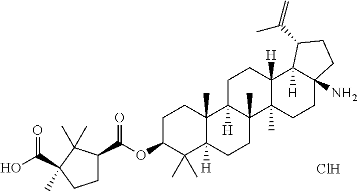 | 3β-O-[(1′S,3′R)- 2′,2′,3′-Trimethyl- cyclopentane-3′- carboxylic acid-1′- carboxyl]-17β-amino- 28-norlup-20(29)-ene hydrochloride | 23.68 (D) | 610.59 |
| 9-3 | 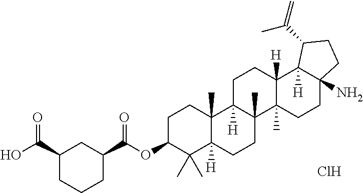 | 3β-O-(cis- Cyclohexane-3′- carboxylic acid-1′- carboxyl)-17β- amino-28-norlup- 20(29)-ene hydrochloride | ¹H NMR (400 MHz, DMSO-d₆): δ [ppm] 7.58 (s, 3H), 4.69 (m, 1H), 4.59 (m, 1H), 4.34 (m, 1H), 3.64 (m, 1H), 3.43 (m, 1H), 2.57 (m, 1H), 2.37 (m, 1H), 2.02 (m, 1H), 1.90-0.68 (m, 48H). | |
| 10-2 | 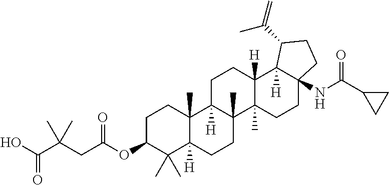 | 17β- (Cyclopropylmethyl- amino)-3β-O-(3′,3′- dimethylsuccinyl)- 28- norlup-20(29)-ene | | 610.52 |
| 10-3 | 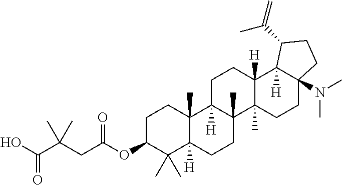 | 3β-0-(3′,3′- Dimethylsuccinyl)- 17β-dimethylamino- 28- norlup-20(29)-ene | | 584.40 |
| 11-9 | 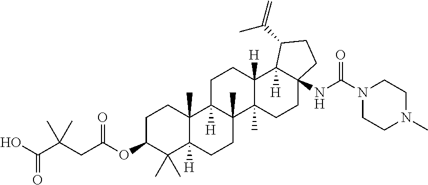 | 3β-O-(3′,3′- Dimethylsuccinyl)- 17β-[(4-methyl- piperazine-1- carbonyl)-amino]-28- norlup-20(29)-ene | 13.07 (F) | 682.85 |
| 11-10 | 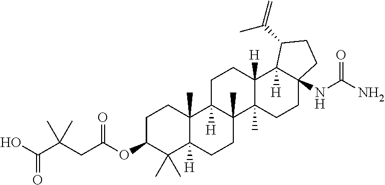 | 3β-O-(3′,3′- Dimethylsuccinyl)- 17β-ureido-28- norlup-20(29)-ene | | 598.51 |
| 11-11 | 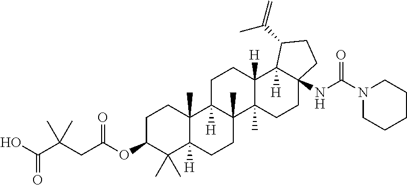 | 3β-O-(3′,3′- Dimethylsuccinyl)- 17β-[(piperidine-1- carbonyl)-amino]-28- norlup-20(29)-ene | 29.37 (E) | 667.76 |
| 11-12 | 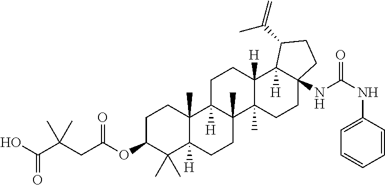 | 3β-O-(3′,3′- Dimethylsuccinyl)- 17β-(N′-phenyl- ureido)-28- norlup-20(29)-ene | 25.21 (E) | 675.75 |
| 11-13 | 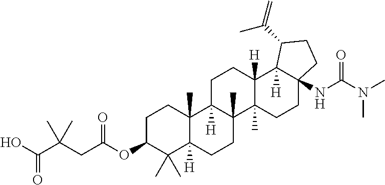 | 3β-O-(3′,3′- Dimethylsuccinyl)- 17β -(N′,N′-dimethyl- ureido)-28- norlup-20(29)-ene | 37.04 (B) | 627.56 |
| 11-14 | 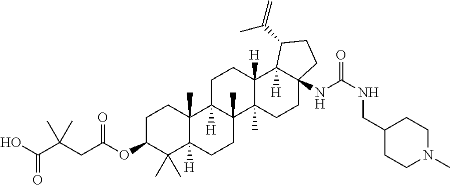 | 3β-O-(3′,3′- Dimethylsuccinyl)- 17β-[N′-(1-methyl- piperidin-4- ylmethyl)-ureido]- 28- norlup-20(29)-ene | 22.82 (D) | 710.94 |
| 11-15 | 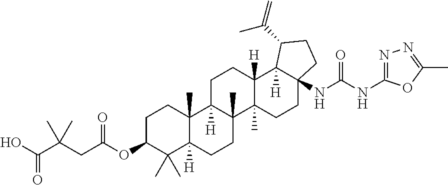 | 3β-O-(3′,3′- Dimethylsuccinyl)- 17β-[3-(5-methyl- [1,3,4]oxadiazol-2- yl)-ureido]-28- norlup-20(29)-ene | 30.40 (B) | 681.99 |
| 11-16 | 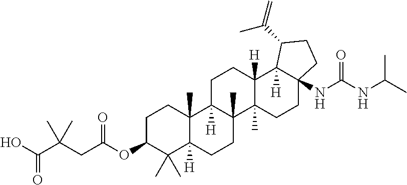 | 3β-O-(3′,3′- Dimethylsuccinyl)- 17β-(N′-isopropyl- ureido)-28- norlup-20(29)-ene | 15.79 (G) | 641.79 |
| 11-17 | 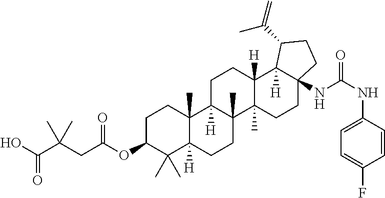 | 3β-O-(3′,3′- Dimethylsuccinyl)- 17β-(N′-4- fluorophenyl-ureido)- 28- norlup-20(29)-ene | 14.27 (G) | 694.06 |
| 11-18 | 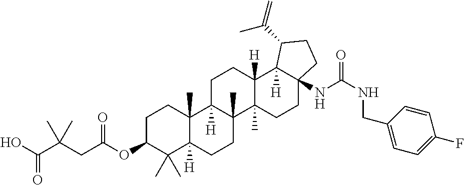 | 3β-O-(3′,3′- Dimethylsuccinyl)- 17β-(N′-4- fluorophenylmethyl- ureido)-28- norlup-20(29)-ene | 5.39 (H) | 708.13 |
| 11-19 | 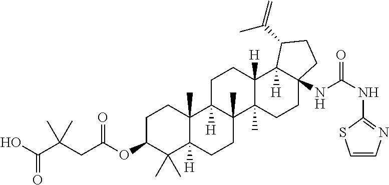 | 3β-O-(3′,3′- Dimethylsuccinyl)- 17β-(N′-thiazol-2-yl- ureido)-28- norlup-20(29)-ene | 12.16 (G) | 682.95 |
| 11-20 | 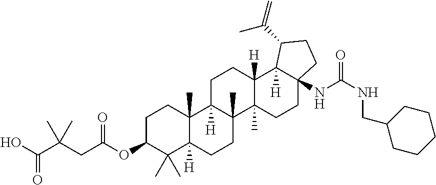 | 3β-O-(3′,3′- Dimethylsuccinyl)- 17β-(N - cyclohexylmethyl- ureido)-28- norlup-20(29)-ene | 15.07 (G) | 696.06 |
| 11-21 | 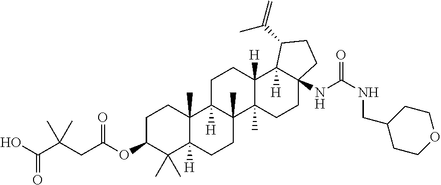 | 3β-O-(3′,3′- Dimethylsuccinyl)- 17β-(N - tetrahydropyran-4- ylmethyl-ureido)-28- norlup-20(29)-ene | 10.64 (G) | 698.10 |
| 11-22 | 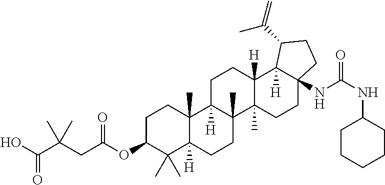 | 3β-O-(3′,3′- Dimethylsuccinyl)- 17β-(N′-cyclohexyl- ureido)-28- norlup-20(29)-ene | 14.40 (G) | 681.96 |
| 11-23 | 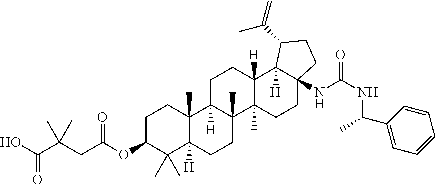 | 3β-O-(3′,3′- Dimethylsuccinyl)- 17β-(N′-(S)-1-phenyl- ethyl-ureido)-28- norlup-20(29)-ene | 15.07 (G) | 704.16 |
| 11-24 | 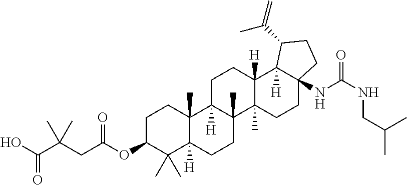 | 3β-O-(3′,3′- Dimethylsuccinyl)- 17β-(N′- isobutylureido)-28- norlup-20(29)-ene | 5.20 (H) | 655.75 |
| 11-25 | 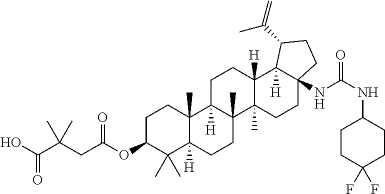 | 3β-O-(3′,3′- Dimethylsuccinyl)- 17β-(N′-4,4- difluorocyclohexyl- ureido)-28- norlup-20(29)-ene | 4.88 (H) | 718.16 |
| 11-26 | 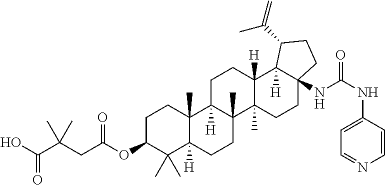 | 3β-O-(3′,3′- Dimethylsuccinyl)- 17β-(N′-pyridin-4-yl- ureido)-28 norlup-20(29)-ene | 11.28 (I) | 676.87 |
| 11-27 | 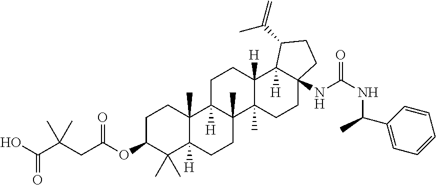 | 3β-O-(3′,3′- Dimethylsuccinyl)- 17β-(N′-(R)-1-phenyl- ethyl-ureido)-28- norlup-20(29)-ene | 13.31 (G) | 704.19 |
| 11-28 | 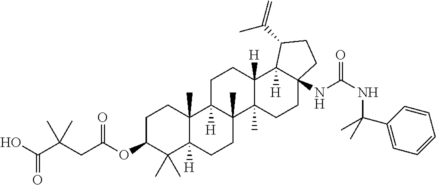 | 3β-O-(3′,3′- Dimethylsuccinyl)- 17β-[N′-(1-methyl-1- phenylethyl)-ureido]- 28- norlup-20(29)-ene | 7.87 (H) | 718.20 |
| 11-29 | 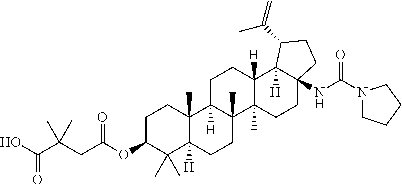 | 3β-O-(3′,3′- Dimethylsuccinyl)- 17β-[(pyrrolidine-1- carbonyl)-amino]-28- norlup-20(29)-ene | 6.11 (H) | 653.74 |
| 13-4 | 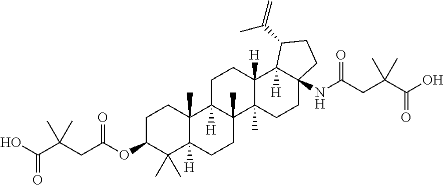 | 3β-O,17β-N-bis(3′,3′- dimethylsuccinyl)- 28-norlup-20(29)-ene | 22.84 (A) | 684.79 |
| 13-5 | 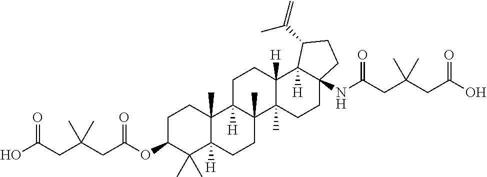 | 3β-O,17β-N-bis(3′,3′- dimethylglutaryl)-28- norlup-20(29)-ene | 29.87 (A) | 712.84 |
| 13-6 | 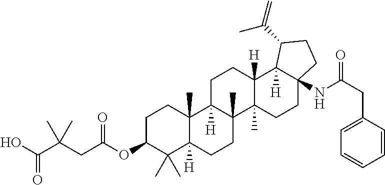 | 3β-O-(3′,3′- Dimethylsuccinyl)- 17β- phenylacetylamino- 28- norlup-20(29)-ene | 31.68 (A) | 674.75 |
| 13-9 | 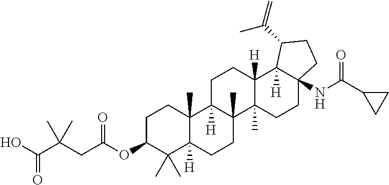 | 17β- (Cyclopropanecarbonyl- amino)-3β-O- (3′,3′- dimethylsuccinyl)- 28-norlup-20(29)-ene | | 624.63 |
| 13-10 | 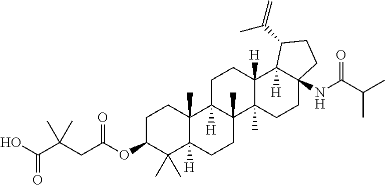 | 3β-O-(3′,3′- Dimethylsuccinyl)- 17β-isobutyrylamino- 28-norlup-20(29)-ene | | 626.66 |
| 13-11 | 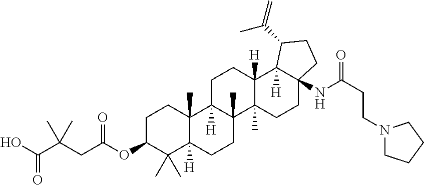 | 3β-O-(3′,3′- Dimethylsuccinyl)- 17β-(3-pyrrolidin-1- yl-propionylamino)- 28- norlup-20(29)-ene | | 681.65 |
| 13-12 | 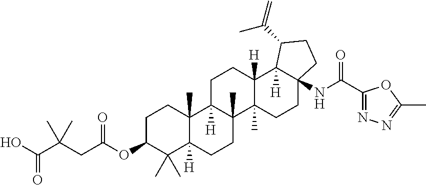 | 3β-O-(3′,3′- Dimethylsuccinyl)- 17β-[(5-methyl- [1,3,4]oxadiazole-2- carbonyl)-amino]-28- norlup-20(29)-ene | 26.49 (A) | 666.89 |
| 13-13 | 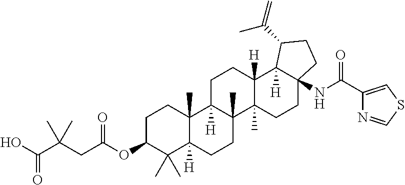 | 3β-O-(3′,3′- Dimethylsuccinyl)- 17β-[(thiazol-4- ylcarbonyl)-amino]- 28-norlup-20(29)-ene | 33.93 (A) | 667.80 |
| 13-14 | 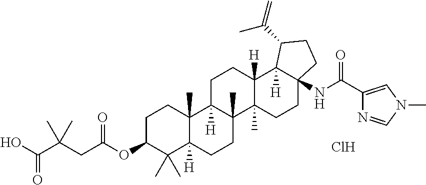 | 3β-O-(3′,3′- Dimethylsuccinyl)- 17β-[(1-methyl-1H- pyrazol-4- ylcarbonyl)-amino]- 28-norlup-20(29)-ene hydrochloride | 10.53 (G) | 664.72 |

| | | | |
|---|---|---|---|
| 13-15 | 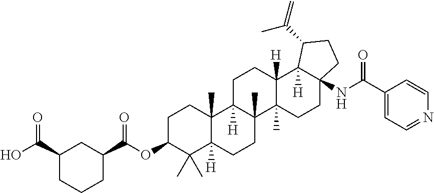 | 3β-O-(cis- Cyclohexane-3′- carboxylic acid-1′- carboxyl)-17β- (pyridin-4- ylcarbonyl)-amino- 28-norlup-20(29)-ene | ¹H NMR (400 MHz, CDCl₃): δ [ppm] 8.70 (d, 2H), 7.50 (d, 2H), 5.80 (s, 1H), 4.66 (s, 1H), 4.59 (s, 1H), 4.40 (m, 1H), 2.72 (m, 1H), 2.58 (m, 1H), 2.38 (m, 1H), 2.20 (m, 2H), 2.00- 1.70 (m, 48H). |

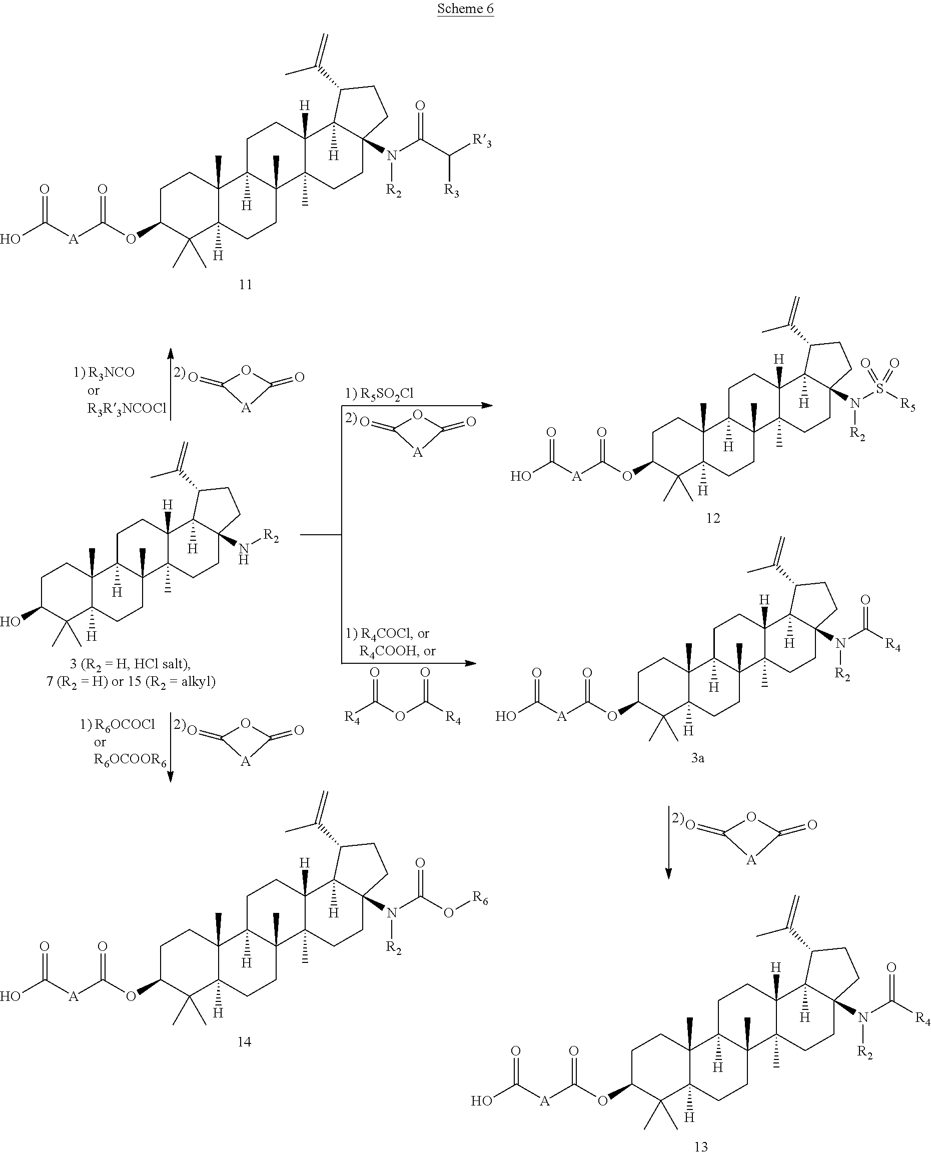

### General Procedures:

Starting from the compound 3 or 7, an alkyl substituent R₂ is introduced by conventional reductive amination with an aldehyde or a ketone (see A. F. Abdel-Magid, et al. J. Org. Chem. (1996), 61, 3849-3862) or by alkylation with an alkyl halide (R₂X) in presence of a base such as TEA, DIPEA or sodium hydride in a solvent such as THF or DMF to give compound 15.

Ureas 11 are made by treatment of compound 3, 7 or 15 with an isocyanate, a carbamoyl chloride or phosgene or triphosgene followed by an amine in a solvent such as toluene or THF. The intermediate is then treated with a base such as DMAP, TEA, DABCO or DIPEA and the appropriate anhydride (2 to 10 equivalents) in solvents such as pyridine, TEA or toluene (0.2-1.0 M) at temperature between 90 to 130° C. for 4 to 24 hours. The mixture is concentrated, washed with aqueous acid and purified by flash column chromatography on silica gel to yield the compound 11.

Sulfonamides 12 are obtained by coupling 3, 7 or 15 with the appropriate sulfonyl chloride in solvents such as THF or DCM and in the presence of a base such as TEA or DIPEA. The intermediate is then treated with a base such as DMAP, TEA, DABCO or DIPEA and the appropriate anhydride (2 to 10 equivalents) in solvents such as pyridine, TEA or toluene (0.2-1.0 M) at temperature between 90 to 130° C. for 4 to 24 hours. The mixture is concentrated, washed with aqueous acid and purified by flash column chromatography on silica gel to yield the compound 12.

Amides 13 are prepared by coupling compound 3, 7 or 15 with the appropriate acyl chloride or mixed anhydride or symmetric anhydride or pre-activated carboxylic acid in solvents such as THF or DCM and in the presence of a base such as TEA or DIPEA. The intermediate is then treated with a base such as DMAP, TEA, DABCO or DIPEA and the appropriate anhydride (2 to 10 equivalents) in solvents such as pyridine, TEA or toluene (0.2-1.0 M) at temperature between 90 to 130° C. for 4 to 24 hours. The mixture is concentrated, washed with aqueous acid and purified by flash column chromatography on silica gel to yield the compound 13.

Carbamates 14 are obtained by reacting compound 3, 7 or 15 with the appropriate chloroformate or symmetric carbonate in solvents such as THF or DCM and in the presence of a base such as TEA or DIPEA. The intermediate is then treated with a base such as DMAP, TEA, DABCO or DIPEA and the appropriate anhydride (2 to 10 equivalents) in solvents such as pyridine, TEA or toluene (0.2-1.0 M) at temperature between 90 to 130° C. for 4 to 24 hours. The mixture is concentrated, washed with aqueous acid and purified by flash column chromatography on silica gel to yield the compound 14.

### 17β-Benzoylamino-3β-O-(3′,3′-dimethylsuccinyl)-28-norlup-20(29)-ene 13-7

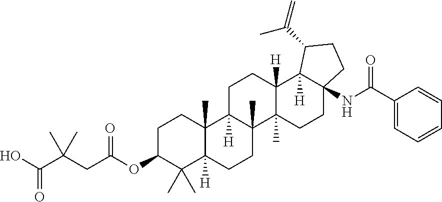

Step 1: To a stirring solution of compound 3 (130 mg, 0.304 mmol) in dry THF (3 mL) is added TEA (0.09 mL, 0.609 mmol) and benzoyl chloride (0.04 mL, 0.335 mmol). The mixture is stirred at room temperature for 1.5 hour, diluted with ethyl acetate, washed twice with water and brine, dried over sodium sulfate. The residue is purified by flash column chromatography on silica gel (ethyl acetate/hexanes 0% to 30%) to yield 17β-benzoylamino-3β-hydroxy-28-norlup-20(29)-ene (154 mg, 95%) as a foam.

¹H NMR (400 MHz, CDCl₃): δ [ppm] 7.71 (m, 2H), 7.48-7.40 (m, 3H), 5.82 (s, 1H), 4.71 (s, 1H), 4.61 (s, 1H), 3.16 (d×d, 1H), 2.80 (d×t, 1H), 2.64 (d×d, 1H), 2.44 (m, 1H), 1.94 (m, 1H), 1.78-0.65 (m, 21H), 1.68 (s, 3H), 1.00 (s, 3H), 0.98 (s, 3H), 0.93 (s, 3H), 0.81 (s, 3H), 0.72 (s, 3H).

Step 2: A stirring solution of 17β-benzoylamino-3β-hydroxy-28-norlup-20(29)-ene (144 mg, 0.270 mmol), DMAP (40 mg, 0.324 mmol) and 2,2-dimethylsuccinic anhydride (208 mg, 1.62 mmol) in dry pyridine (3 mL) is heated overnight at 120° C. Another 6 equivalents of anhydride is added and heating is continued for 7 hours. The mixture is concentrated to dryness and the residue is diluted in ethyl acetate, washed twice with HCl 1N, water and brine, dried over sodium sulfate and concentrated to dryness. The residue is purified by flash column chromatography on silica gel (ethyl acetate/hexanes 10% to 70%) followed by crystallization in ethyl acetate/hexanes (1:3) to yield the title compound 13-7 as a white solid (30 mg, 16%).

¹H NMR (400 MHz, CDCl₃): δ [ppm] 7.72 (m, 2H), 7.51-7.41 (m, 3H), 5.81 (s, 1H), 4.71 (d, 1H), 4.62 (t, 1H), 4.47 (d×d, 1H), 2.82 (d×t, 1H), 2.65 (m, 1H), 2.64 (d, 1H), 2.54 (d, 1H), 2.44 (m, 1H), 1.95 (m, 1H), 1.75-0.75 (m, 21H), 1.69 (s, 3H), 1.29 (s, 3H), 1.27 (s, 3H), 1.01 (s, 3H), 0.99 (s, 3H), 0.83 (s, 3H), 0.82 (s, 3H), 0.78 (s, 3H).

LC/MS: m/z=660.71 (M+H⁺).

HPLC (Method A): t_{R}=34.04 min.

Table 2 of compounds illustrates some of the compounds of the present invention which are synthesized using the procedures described in scheme 6.

Retention time (t_{R}) for each compound are measured using the standard analytical HPLC methods described above.

TABLE 2

| | | | t_{R}(min) | |
|---|---|---|---|---|
| Cpd # | Structure | Name | (Method) | M + H⁺ |
| 13-8 | 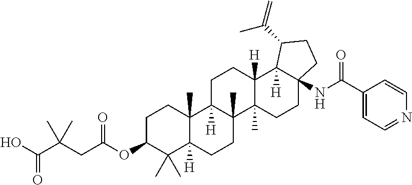 | 3β-O-(3′,3′- Dimethylsuccinyl)-17β- (pyridin-4-ylcarbonyl)- amino-28-norlup- 20(29)-ene | 17.80 (A) | 661.57 |

Ureas 11 and carbamates 14 can also be prepared from the isocyanate 2 as described in scheme 7.

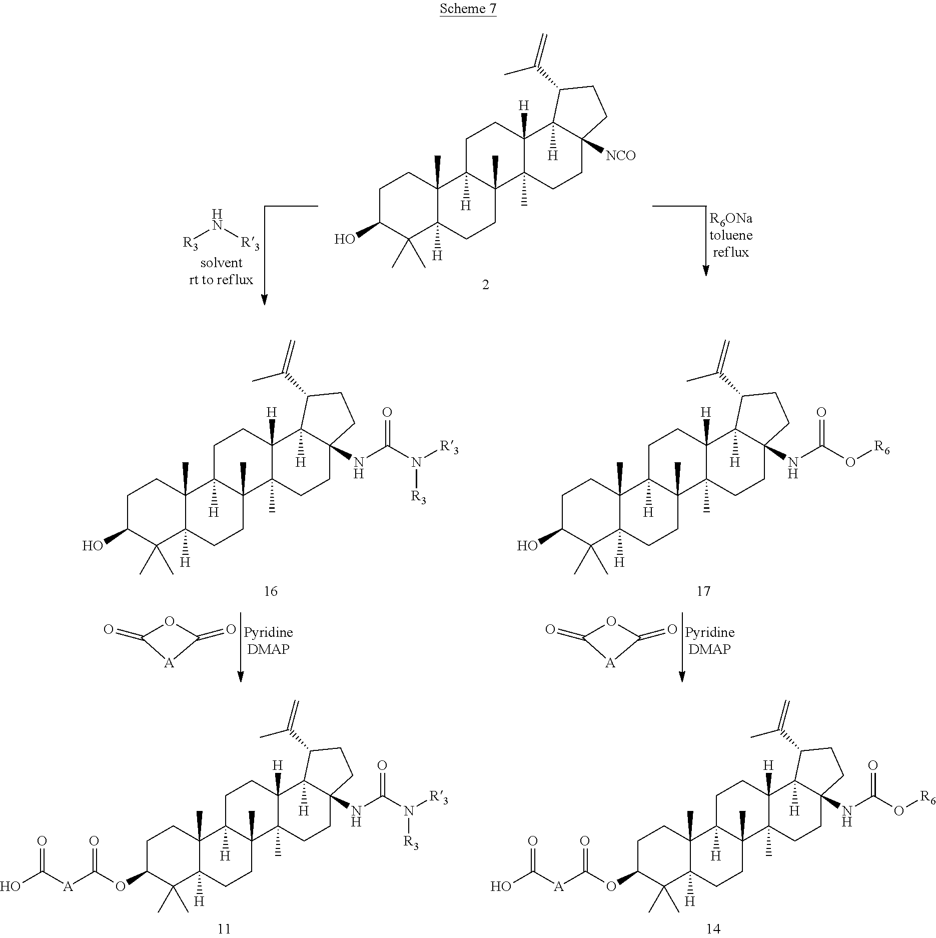

### General Procedure for the Synthesis of Ureas 11

Step 1: A solution of isocyanate 2 and the desired amine in solvents such as benzene, toluene or chloroform is stirred for 4 to 20 hours at room temperature or under reflux. The residue obtained is purified by flash chromatography on silica gel to afford the desired urea 16.

Step 2: A solution of urea 16, a base such as DMAP, TEA, DABCO or DIPEA and the appropriate anhydride (2 to 10 equivalents) in solvents such as pyridine, TEA or toluene (0.2-1.0 M) is heated from 90 to 130° C. for 4 to 24 hours. The mixture is concentrated, washed with aqueous acid and purified by flash chromatography on silica gel to yield compound 11.

### General Procedure for the Synthesis of Carbamates 14

Step 1: To a stirring solution of isocyanate 2 in solvents such as toluene or benzene is added the desired sodium alcoholate (1 to 5 equivalents). The resulting mixture is stirred for 2 to 4 hours under reflux. After standard acidic workup, the residue obtained is purified by flash chromatography on silica gel to afford the desired carbamate 17.

Step 2: A stirring solution of carbamate 17, a base such as DMAP, TEA, DABCO or DIPEA and the appropriate anhydride (2 to 10 equivalents) in solvents such as pyridine, TEA or toluene (0.2-1.0 M) is heated from 90 to 130° C. for 4 to 24 hours. The mixture is concentrated, washed with aqueous acid and purified by flash chromatography on silica gel to yield compound 14.

Table 3 illustrates some intermediates which are synthesized using the procedures described in Scheme 6 and 7.

| Cpd # | Structure | Name | NMR |
|---|---|---|---|
| 3a-1 | 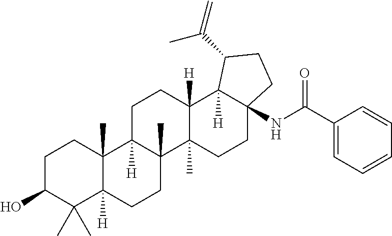 | 17β-benzoylamino- 3β-hydroxy-28- norlup-20(29)-ene | ¹H NMR (400 MHz, CDCl₃): δ [ppm] 7.71 (m, 2H), 7.48-7.40 (m, 3H), 5.82 (s, 1H), 4.71 (s, 1H), 4.61 (s, 1H), 3.16 (d x d, 1H), 2.80 (d x t, 1H), 2.64 (d x d, 1H), 2.44 (m, 1H), 1.94 (m, 1H ), 1.78-0.65 (m, 21H), 1.68 (s, 3H), 1.00 (s, 3H), 0.98 (s, 3H), 0.93 (s, 3H), 0.81 (s, 3H), 0.72 (s, 3H). |
| 3a-2 | 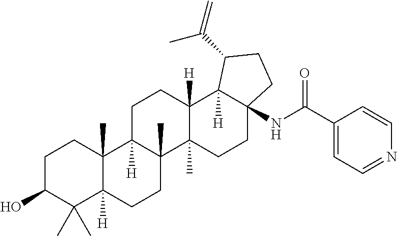 | 3β-hydroxy-17β- (pyridin-4- ylcarbonyl)-amino- 28-norlup-20(29)-ene | ¹H NMR (400 MHz, DMSO-d₆): δ [ppm] 8.67 (d x d, 2H), 7.59 (d x d, 2H), 7.32 (s, 1H), 4.66 (d, 1H), 4.55 (t, 1H), 4.24 (d, 1H), 2.90 (m, 2H), 2.68 (m, 1H), 2.40 (m, 1H), 2.09 (m, 1H), 1.76 (m, 1H), 1.68-0.60 (m, 20 H), 1.63 (s, 3H), 0.98 (s, 3H), 0.91 (s, 3H), 0.84 (s, 3H), 0.76 (s, 3H), 0.62 (s, 3H). |
| 16-1 | 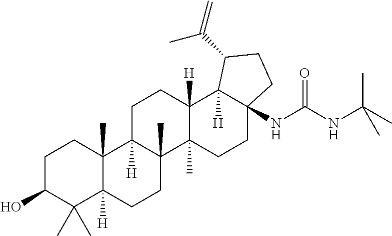 | 3β-hydroxy-17β-[N′- (tert-butyl)ureido]- 28-norlup-20(29)-ene | ¹H NMR (400 MHz, DMSO-d₆): δ [ppm] 5.70 (s, 1H), 5.22 (s, 1H), 4.66 (s, 1H), 4.56 (s, 1H), 4.26 (d, 1H), 2.96 (m, 1H), 2.25 (m, 1H), 1.75 (m, 2H), 1.65-0.80 (m, 22H), 1.63 (s, 3H), 1.18 (s, 9H), 0.96 (s, 3H), 0.88 (s, 3H), 0.86 (s, 3H), 0.77 (s, 3H), 0.64 (s, 3H). |
| 16-2 | 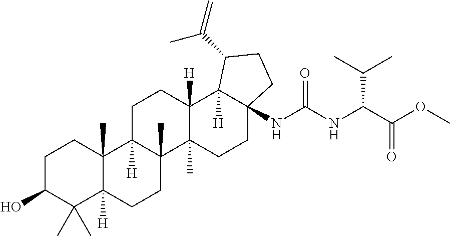 | 3β-hydroxy-28- norlup-20(29)-ene- 17β-yl-N-carbamoyl- L-valine | ¹H NMR (400 MHz, DMSO-d₆): δ [ppm] 6.20 (d, 1H), 5.66 (s, 1H), 4.68 (d, 1H), 4.57 (s, 1H), 4.26 (d, 1H), 4.03 (m, 1H), 3.60 (s, 3H), 2.96 (m, 1H), 2.55 (m, 1H), 2.17 (m, 1H), 1.94 (m, 1H), 1.76 (m, 2H), 1.64-0.61 (m, 21H), 1.63 (s, 3H), 0.99 (s, 3H), 0.89 (s, 3H), 0.85 (d, 3H), 0.82 (d, 3H), 0.81 (s, 3H), 0.78 (s, 3H), 0.64 (s, 3H). |
| 16-3 | 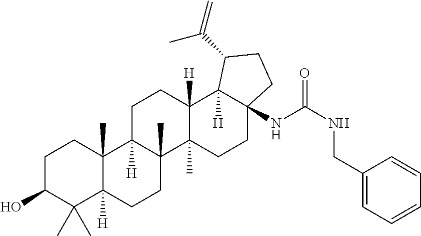 | 3β-hydroxy-17β-[N′- (benzyl)ureido]-28- norlup-20(29)-ene | ¹H NMR (400 MHz, DMSO-d₆): δ [ppm] 7.30 (m, 2H), 7.20 (m, 3H), 6.30 (t, 1H), 5.50 (s, 1H), 4.66 (d, 1H), 4.57 (m, 1H), 4.26 (d, 1H), 4.16 (d x d, 2H), 2.95 (m, 1H), 2.53 (m, 2H), 2.26 (m, 1H), 1.75 (m, 2H), 1.65-0.60 (m, 20H), 1.64 (s, 3H), 0.98 (s, 3H), 0.90 (s, 3H), 0.86 (s, 3H), 0.77 (s, 3H), 0.64 (s, 3H). |
| 16-4 | 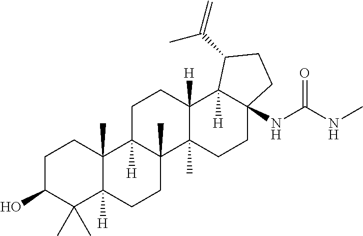 | 3β-hydroxy-17β-[N′- (methyl)ureido]-28- norlup-20(29)-ene | ¹H NMR (400 MHz, CDCl₃): δ [ppm] 4.69 (d, 1H), 4.60 (d x d, 1H), 4.24 (br s, 1H), 3.18 (d x d, 1H), 2.78 (s, 3H), 2.54 (m, 1H), 2.43 (m, 2H), 1.95 (m, 1H), 1.68-0.64 (m, 21H), 1.67 (s, 3H), 1.00 (s, 3H), 0.96 (s, 6H), 0.82 (s, 3H), 0.75 (s, 3H). |
| 17-1 | 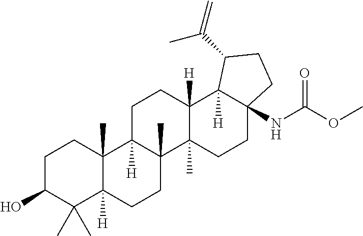 | 3β-hydroxy-17β- methoxycarbonylamino- 28-norlup-20(29)- ene | ¹H NMR (400 MHz, CDCl₃): δ [ppm] 4.69 (d, 1H), 4.59 (d x d, 1H), 4.49 (s, 1H), 3.62 (s, 3H), 3.17 (d x d, 1H), 2.45 (m, 3H), 1.94 (m, 1H), 1.66 (s, 3H), 1.66-0.63 (m, 21H), 0.99 (s, 3H), 0.95 (s, 3H), 0.94 (s, 3H), 0.81 (s, 3H), 0.75 (s, 3H). |
| 17-2 | 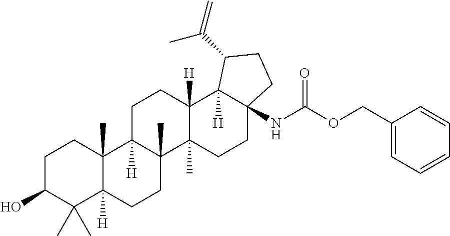 | 3β-hydroxy-17β- benzyloxycarbonylamino- 28-norlup-20(29)- ene | ¹H NMR (400 MHz, CDCl₃): δ [ppm] 7.32 (m, 5H), 5.06 (s, 2H), 4.69 (m, 2H), 4.59 (m, 2H), 3.17 (d x d, 1H), 2.56 (m, 1H), 2.40 (m, 2H), 1.94 (m, 1H), 1.67 (s, 3H), 1.67-0.60 (m, 21H), 0.98 (s, 3H), 0.96 (s, 3H), 0.95 (s, 3H), 0.81 (s, 3H), 0.75 (s, 3H). |

### 3β-O-(3′,3′-Dimethylsuccinyl)-17β-[N′-(tert-butyl)ureido]-28-norlup-20(29)-ene 11-2

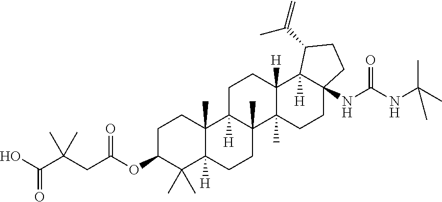

Step 1: To a stirring solution of compound 2 (473 mg, 1.04 mmol) in dry benzene (10 mL) is added tert-butylamine (0.44 mL, 4.16 mmol). The resulting mixture is stirred for 20 hours under reflux, and concentrated to dryness. The residue obtained is purified by flash chromatography on silica gel (ethyl acetate/hexanes 0% to 40%, followed by ethyl acetate 100%) to afford 3β-hydroxy-17β-[N′-(tert-butyl)ureido]-28-norlup-20(29)-ene 16-1 (478 mg, 75%) as a white solid.

Step 2: A stirring solution of compound 16-1 (203 mg, 0.385 mmol), DMAP (56 mg, 0.462 mmol) and 2,2-dimethylsuccinic anhydride (150 mg, 1.1 mmol) in dry pyridine (4 mL) is heated for 4 hours at 120° C. Another 150 mg, 1.1 mmol of anhydride is added and heating is continued overnight. The mixture is concentrated to dryness and the residue is diluted in ethyl acetate, washed twice with HCl 1N, water and brine, dried over sodium sulfate and concentrated to dryness. The residue is purified by flash chromatography on silica gel (ethyl acetate/hexanes 10% to 60%) to yield the title compound 11-2 as a white solid (33 mg, 13%).

¹H NMR (400 MHz, CDCl₃): δ [ppm] 7.00 (br s, 1H), 4.71 (s, 1H), 4.62 (s, 1H), 4.46 (d×d, 1H), 4.17 (br s, 1H), 2.88 (d, 1H), 2.78 (m, 1H), 2.43 (d, 1H), 2.35 (m, 2H), 1.90 (m, 1H), 1.75-0.90 (m, 21H), 1.69 (s, 3H), 1.38 (s, 9H), 1.24 (s, 3H), 1.19 (s, 3H), 1.06 (s, 3H), 0.97 (s, 3H), 0.84 (s, 3H), 0.83 (s, 3H), 0.79 (s, 3H).

LC/MS: m/z=655.78 (M+H⁺).

HPLC (Method A): t_{R}=30.81 min.

### 3β-O-(3′,3′-Dimethylsuccinyl)-28-norlup-20(29)-ene-17β-yl-N-carbamoyl-L-valine methyl ester 11-3

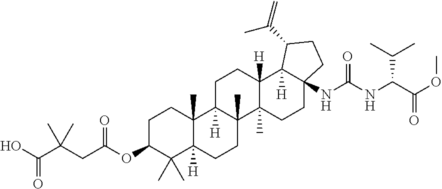

Step 1: To a stirring solution of L-valine methyl ester hydrochloride (144 mg, 0.86 mmol) in dry chloroform (3.3 mL) is added TEA (0.15 mL, 1.06 mmol). A solution of isocyanate 2 (300 mg, 0.66 mmol) in dry chloroform (3.3 mL) is added. The resulting mixture is stirred overnight under reflux. L-valine methyl ester hydrochloride (53 mg, 0.319 mmol) and TEA (0.74 mL, 0.53 mmol) in dry chloroform are mixed together and added to the reaction mixture. The resulting mixture is reflux for 24 hours, diluted with DCM, washed twice with HCl 1N, water and brine, dried over sodium sulfate and concentrated to dryness. The residue is purified by flash chromatography on silica gel (ethyl acetate/hexanes 0% to 30%) to afford 3β-hydroxy-28-norlup-20(29)-ene-17β-yl-N-carbamoyl-L-valine methyl ester 16-2 (349 mg, 90%) as a white solid.

Step 2: A stirring solution of compound 16-2 (121 mg, 0.207 mmol), DMAP (30 mg, 0.248 mmol) and 2,2-dimethylsuccinic anhydride (80 mg, 0.620 mmol) in dry pyridine (2 mL) is heated for 6 hours at 120° C. Another 80 mg (0.62 mmol) of anhydride is added and heating is continued overnight. The mixture is concentrated to dryness and the residue is diluted in ethyl acetate, washed twice with HCl 1N, water and brine, dried over sodium sulfate and concentrated to dryness. The residue is purified by flash chromatography on silica gel (ethyl acetate/hexanes 20% to 60%) to give the title compound 11-3 as a glass (127 mg, 86%).

¹H NMR (400 MHz, CDCl₃): δ [ppm] 4.70 (d, 1H), 4.60 (s, 1H), 4.46 (m, 2H), 3.75 (s, 3H), 2.80 (m, 1H), 2.63 (m, 1H), 2.45 (m, 2H), 2.30 (d×d, 1H), 2.03 (m, 1H), 1.85 (m, 1H), 1.70-0.80 (m, 20H), 1.67 (s, 3H), 1.26 (s, 3H), 1.23 (s, 3H), 1.03 (s, 3H), 0.99 (s, 3H), 0.98 (s, 3H), 0.81 (s, 3H), 0.79 (s, 6H).

LC/MS: m/z=713.77 (M+H⁺).

### 3β-O-(3′,3′-Dimethylsuccinyl)-28-norlup-20(29)-ene-17β-yl-N-carbamoyl-L-valine 11-4

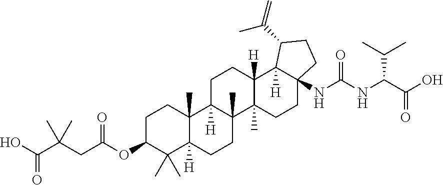

To a stirring solution of compound 11-3 (126 mg, 0.177 mmol) in THF/methanol (1:1 mixture, 5 mL) is added aqueous KOH 10% (1 mL). The resulting mixture is heated at 50° C. for 2 hours, cooled down and concentrated. The slightly pink solid is suspended in water (3 mL) with stirring and acidified to pH 3 by slow addition of HCl 6N. A white solid precipitates and is collected by filtration to give the title compound 11-4 (87 mg, 71%) as a white solid.

¹H NMR (400 MHz, DMSO-d₆): δ [ppm] 12.25 (br s, 2H), 6.12 (d, 1H), 5.67 (s, 1H), 4.68 (d, 1H), 4.57 (s, 1H), 4.35 (d×d, 1H), 3.98 (d×d, 1H), 2.58 (m, 1H), 2.54-2.45 (m, 2H), 2.19 (m, 1H), 1.95 (m, 1H), 1.80 (m, 1H), 1.75 (m, 1H), 1.60-0.90 (m, 20H), 1.64 (s, 3H), 1.15 (s, 3H), 1.14 (s, 3H), 0.99 (s, 3H), 0.90 (s, 3H), 0.85 (s, 3H), 0.82 (s, 3H), 0.81 (s, 3H), 0.77 (s, 3H).

LC/MS: m/z=699.77 (M+H⁺).

HPLC (Method A): t_{R}=20.85 min.

### 3β-O-(3′,3′-Dimethylsuccinyl)-17β-methoxycarbonylamino-28-norlup-20(29)-ene 14-1

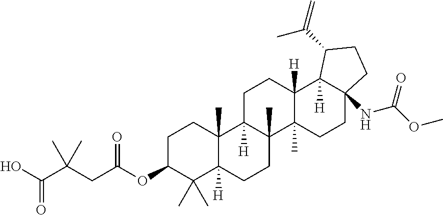

Step 1: To a stirring solution of isocyanate 2 (142 mg, 0.313 mmol) in dry toluene (6 mL) is added sodium methoxide in methanol (0.215 mL, 0.939 mmol). The resulting mixture is stirred for 2.5 hours under reflux, cooled down and diluted with ethyl acetate, washed with HCl 1N, water and brine, dried over sodium sulfate and concentrated to dryness. The residue obtained is purified by flash chromatography on silica gel (ethyl acetate/hexanes 5% to 30%) to afford 3β-hydroxy-17β-methoxycarbonylamino-28-norlup-20(29)-ene 17-1 (118 mg, 81%) as a foam.

Step 2: A stirring solution of compound 17-1 (112 mg, 0.232 mmol), DMAP (28 mg, 0.232 mmol) and 2,2-dimethylsuccinic anhydride (89 mg, 0.695 mmol) in dry pyridine (4 mL) is heated for 4 hours at 120° C. Another 90 mg (0.7 mmol) of anhydride is added and heating is continued overnight. The mixture is concentrated to dryness and the residue is diluted in ethyl acetate, washed twice with HCl 1N, water and brine, dried over sodium sulfate and concentrated to dryness. The residue is purified by flash chromatography on silica gel (ethyl acetate/hexanes 10% to 30%) to yield the title compound 14-1 as a white solid (87 mg, 61%).

¹H NMR (400 MHz, DMSO-d₆): δ [ppm] 12.16 (s, 1H), 6.33 (s, 1H), 4.64 (d, 1H), 4.54 (s, 1H), 4.35 (d×d, 1H), 3.47 (s, 3H), 2.73 (m, 1H), 2.53 (d, 1H), 2.45 (d, 1H), 2.42 (m, 1H), 2.15 (m, 1H), 1.92 (m, 1H), 1.75 (m, 1H), 1.70-0.90 (m, 20H), 1.62 (s, 3H), 1.15 (s, 3H), 1.14 (s, 3H), 0.95 (s, 3H), 0.91 (s, 3H), 0.89 (s, 3H), 0.84 (s, 3H), 0.80 (s, 3H).

LC/MS: m/z=614.72 (M+H⁺).

HPLC (Method A): t_{R}=31.62 min.

Table 4 of compounds illustrates some of the compounds of the present invention which are synthesized using the procedures described in scheme 7.

Retention time (t_{R}) for each compound are measured using the standard analytical HPLC methods described above.

TABLE 4

| | | | t_{R}(min) | |
|---|---|---|---|---|
| Cpd # | Structure | Name | (Method) | M + H⁺ |
| 11-5 | 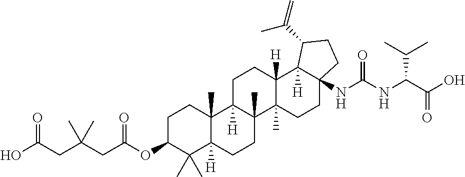 | 3β-O-(3′,3′- Dimethylglutaryl)-28- norlup-20(29)-ene- 17β-yl-N- carbamoyl-L-valine | 23.60 (A) | 713.79 |
| 11-6 | 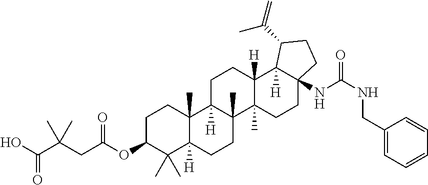 | 3β-O- (3′,3′- Dimethylsuccinyl)- 17β-[N′- (benzyl)ureido]-28- norlup-20(29)-ene | 29.21 (A) | 689.79 |
| 11-7 | 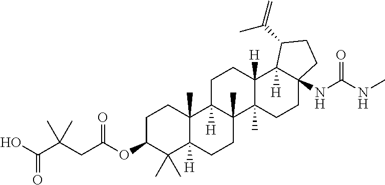 | 3β-O- (3′,3′- Dimethylsuccinyl)- 17β-[N′- (methyl)ureido]-28- norlup-20(29)-ene | 21.02 (A) | 613.72 |
| 14-2 | 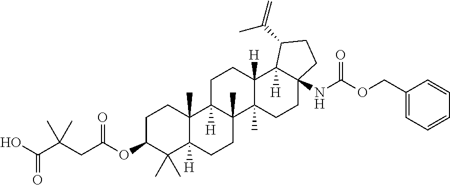 | 3β-O- (3′,3′- Dimethylsuccinyl)- 17β- benzyloxycarbonylamino- 28-norlup-20(29)-ene | 37.67 (A) | 690.74 |

The double bond at C20(29) can be reduced at any stage by standard hydrogenation conditions when the substituents are stable to such conditions.

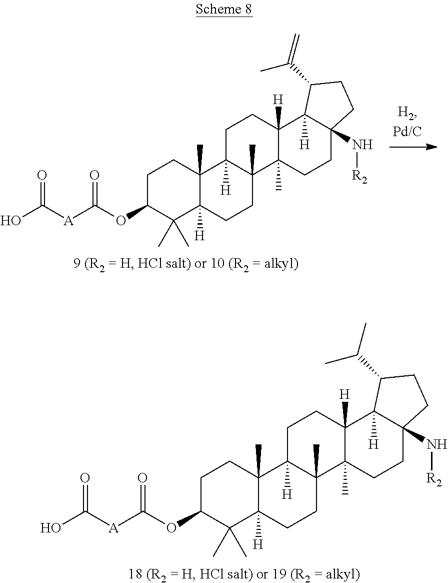

### General Procedure:

A solution of compound 9 or 10 and Pd/C 10% in solvent such as methanol is stirred for 1 to 24 hours under hydrogen atmosphere at room temperature. The reaction mixture is filtered through Celite and concentrated to dryness. The residue obtained is purified by flash chromatography on silica gel to give the compound 18 or 19, respectively.

### 17β-Amino-3β-O-(3′,3′-dimethylsuccinyl)-28-norlupane hydrochloride 18-1

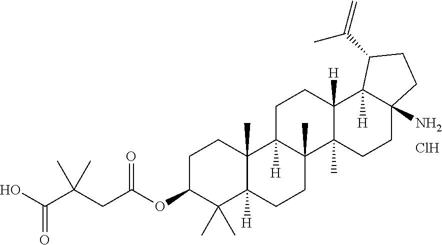

A solution of compound 9-1 (113 mg, 0.191 mmol) and Pd/C 10% (20 mg) in methanol (3 mL) is stirred for 11 hours under hydrogen atmosphere at room temperature. The reaction mixture is filtered through Celite and concentrated to dryness. The residue obtained is purified by flash chromatography on silica gel (methanol/DCM 0% to 15%) to give the title compound 18-1 (53 mg, 50%) as a white solid.

¹H NMR (400 MHz, CD₃OD): δ [ppm] 4.46 (d×d, 1H), 2.62 (d, 1H), 2.53 (d, 1H), 2.0-1.25 (m, 25H), 1.24 (s, 3H), 1.23 (s, 3H), 1.11 (s, 3H), 1.03 (m, 1H), 1.02 (s, 3H), 0.91 (s, 3H), 0.90 (d, 3H), 0.86 (s, 6H), 0.81 (d, 3H).

LC/MS: m/z=558.67 (M+H⁺).

HPLC (Method C) t_{R}=31.32 min.

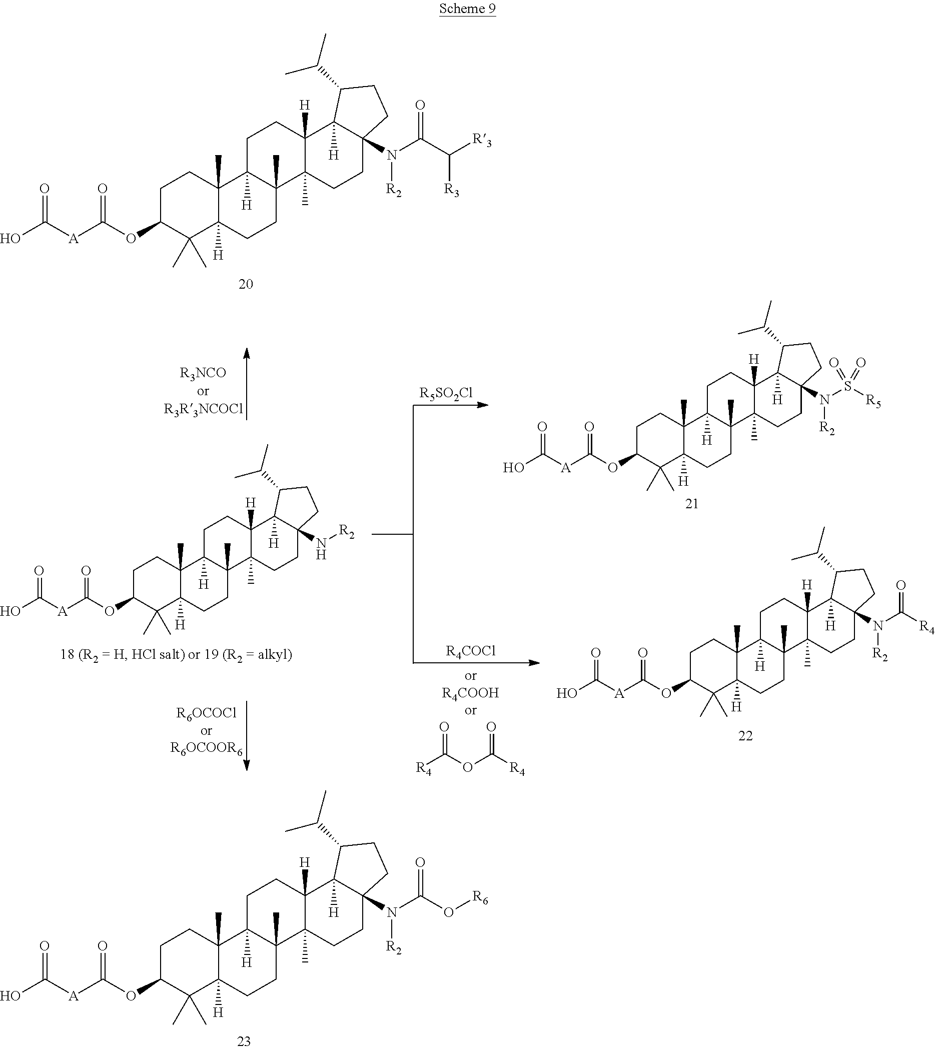

### General Procedure:

Ureas 20 are made by treatment of compound 18 or 19 with an isocyanate, a carbamoyl chloride or phosgene or triphosgene followed by an amine in a solvent such as toluene or THF.

Sulfonamides 21 are obtained by coupling 18 or 19 with the appropriate sulfonyl chloride in solvents such as THF or DCM and in the presence of a base such as TEA or DIPEA.

Amides 22 are prepared by coupling compound 18 or 19 with the appropriate acyl chloride or mixed anhydride or symmetric anhydride or pre-activated carboxylic acid in solvents such as THF or DCM and in the presence of a base such as TEA or DIPEA.

Carbamates 23 are obtained by reacting compound 18 or 19 with the appropriate chloroformate or symmetric carbonate in solvents such as THF or DCM and in the presence of a base such as TEA or DIPEA.

### 3β-O-(3′,3′-Dimethylsuccinyl)-17β-methoxycabonylamino-28-norlupane 23-1

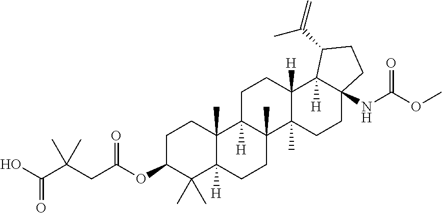

To a stirring solution of compound 18-1 (22 mg, 0.037 mmol) in dry THF (1 mL) is added TEA (0.01 mL, 0.073 mmol) and methyl chloroformate (0.04 mL, 0.055 mmol). The mixture is stirred at room temperature overnight, diluted with ethyl acetate, washed twice with water and brine, dried over sodium sulfate. The residue is purified by flash column chromatography on silica gel (ethyl acetate/hexanes 0% to 20%) to yield the title compound 23-1 (10 mg, 46%) as a white solid.

¹H NMR (400 MHz, CDCl₃): δ [ppm] 4.48 (d×d, 1H), 4.44 (br s, 1H), 3.60 (s, 3H), 2.62 (d, 1H), 2.57 (d, 1H), 2.50 (m, 1H), 2.27 (m, 1H), 1.83 (m, 1H), 1.71-0.80 (m, 23H), 1.31 (s, 3H), 1.31 (s, 3H), 0.98 (s, 3H), 0.91 (s, 3H), 0.85 (d, 3H), 0.84 (s, 3H), 0.82 (s, 3H), 0.81 (s, 3H), 0.74 (d, 3H).

LC/MS: m/z=615.5 (M+H⁺).

Table 5 of compounds illustrates some of the compounds of the present invention which are synthesized using the procedures described in scheme 9.

Retention time (t_{R}) for each compound are measured using the standard analytical HPLC methods described above.

TABLE 5

| | | | t_{R}(min) | |
|---|---|---|---|---|
| Cpd # | Structure | Name | (Method) | M + H⁺ |
| 22-1 | 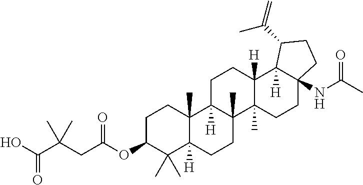 | 3β-O-(3′,3′- Dimethylsuccinyl)- 17β-acetylamino-28- norlupane | 36.59 (B) | 600.66 |

### HIV Replication Activity

HIV-1 Replication in MT2 cell line with and without 30% human serum: The cells are infected at a Multiciplicity of Infection (MOI) of 0.5 for 3 h and then washed twice with complete media to remove residual virus. Cells are then resuspended at 0.5×10⁶/ml in complete medium (RPMI, 10% FBS, 1% sodium pyruvate), and seeded into 96-well plates (6.25×10⁴/well). The cells are cultured in the presence or absence of various concentrations of test compounds in serial dilutions for 3 days at 37° C. The test compounds are serially diluted in complete medium supplemented or not with 30% human serum. After 3 days, 100 μL of cultured medium with cells are replaced with 120 μL of freshly diluted test compounds in complete medium containing or not 30% Human serum. The level of HIV-1 replication is determined at days 5 after infection by the presence of viral RT activity in harvested supernatant fluid. The IC₅₀ values for the virus replication are determined by using GRAPHPAD PRISM software.

PBMCs are separated from healthy donors' blood by standard density gradient centrifugation, resuspended at a cell density of 1.5×10⁶ cells/ml in culture medium containing 2 μg/mL of phytohaemagglutinin (PHA), and thereafter incubated for 3 days at 37° C. in a humidified 5% CO₂ atmosphere. The PHA-stimulated PBMCs are adjusted at a concentration of 5×10⁶/mL and then infected with HIV-1_{IIIB} at a MOI of 5.0 for 3 hours at 37° C. in a humidified 5% CO₂ atmosphere and then washed to remove any residual virus. Thereafter, cells are resuspended in culture medium supplemented with interleukin-2 (IL-2) at a concentration of 50 units/mL (2×) and seeded at a density of 0.2×10⁶ cells/well into 96-well plates in the absence or presence of various concentrations of the test compound. Then, infected-cells are cultured for 4 days at 37° C. in a humidified 5% CO₂ atmosphere in the absence or presence of 30% human serum after which an aliquot of cultured medium supernatant is replaced with fresh medium supplemented with human serum (when necessary) containing the serially diluted test compound. The IC₅₀ values for the virus replication are determined at day 6 post-infection by measuring the reverse transcriptase activity in the harvested supernatant by using GRAPHPAD PRISM software.

The IC50 of the compounds tested in accordance with the HIV replication activity assay MT2 (HIV_{IIIB}) with or without human serum are represented in Table 6.

TABLE 6

| | | MT2 (HIV_{IIIB}) with |
|---|---|---|
| | MT2 (HIV_{IIIB}) | human serum |
| Cpd# | IC50 range | IC50 range |
| 8-1 | +++ | |
| 8-2 | | + |
| 8-3 | | + |
| 9-1 | +++ | +++ |
| 9-2 | | +++ |
| 9-3 | | +++ |
| 10-2 | | +++ |
| 10-3 | | +++ |
| 11-1 | +++ | +++ |
| 11-2 | +++ | +++ |
| 11-4 | +++ | |
| 11-5 | +++ | |
| 11-6 | +++ | +++ |
| 11-7 | +++ | +++ |
| 11-8 | +++ | +++ |
| 11-9 | +++ | +++ |
| 11-10 | +++ | +++ |
| 11-11 | | +++ |
| 11-12 | | +++ |
| 11-13 | | +++ |
| 11-14 | | +++ |
| 11-15 | | +++ |
| 11-16 | | ++ |
| 11-17 | | +++ |
| 11-18 | | +++ |
| 11-19 | | +++ |
| 11-20 | | +++ |
| 11-21 | | +++ |
| 11-22 | | +++ |
| 11-23 | | +++ |
| 11-24 | | +++ |
| 11-25 | | +++ |
| 11-26 | | +++ |
| 11-27 | | +++ |
| 11-28 | | +++ |
| 11-29 | | +++ |
| 12-1 | +++ | +++ |
| 13-1 | +++ | +++ |
| 13-2 | +++ | |
| 13-3 | +++ | +++ |
| 13-4 | +++ | |
| 13-5 | +++ | |
| 13-6 | +++ | +++ |
| 13-7 | +++ | |
| 13-8 | +++ | ++ |
| 13-9 | +++ | +++ |
| 13-10 | +++ | +++ |
| 13-11 | +++ | +++ |
| 13-12 | | +++ |
| 13-13 | | +++ |
| 13-14 | +++ | |
| 13-15 | | + |
| 14-1 | +++ | +++ |
| 14-2 | +++ | |
| 18-1 | +++ | |
| 22-1 | +++ | +++ |
| 23-1 | +++ | |

When the compounds are tested more than once, the average IC50 is provided.
MT2 (HIV_{IIIB}) IC50 with or without human serum
   +>1000 nM
   ++200-999 nM
   +++<199 nM

The preceding examples could be repeated with similar success by substituting the generically or specifically described reactants and/or operating conditions of this invention for those used in the preceding examples.

While the invention has been illustrated with respect to the production and of particular compounds, it is apparent that variations and modifications of the invention can be made without departing from the spirit or scope of the invention.

1. A compound of formula (I): 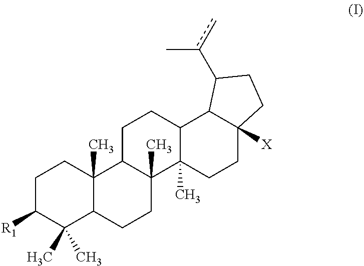
wherein
R¹ is 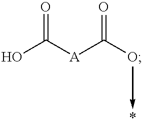
A is C₁₋₈ alkyl, C₂₋₈ alkenyl, or —(CH₂)₁₋₂O(CH)₁₋₂—;
X is 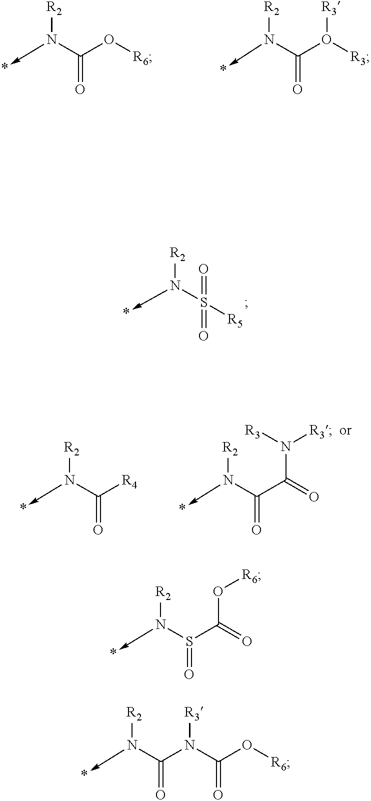
R₂ is H, C₁₋₁₂ alkyl which is unsubstituted or substituted one or more times by R₁₀, C₂₋₁₂ alkenyl which is unsubstituted or substituted one or more times by R₁₀, or C₂₋₁₂ alkynyl which is unsubstituted or substituted one or more times by R₁₀;
R₃ and R₃′ are each independently H, C₁₋₁₂ alkyl which is unsubstituted or substituted one or more times by R₁₀, C₂₋₁₂ alkenyl which is unsubstituted or substituted one or more times by R₁₀, C₂₋₁₂ alkynyl which is unsubstituted or substituted one or more times by R₁₀, C₆₋₁₄ aryl which is unsubstituted or substituted one or more times by R₁₁, C₇₋₁₆ aralkyl which is unsubstituted or substituted one or more times by R₁₁, 5-12 member heteroaryl which is unsubstituted or substituted one or more times by R₁₁, 6-18 member heteroaralkyl which is unsubstituted or substituted one or more times by R₁₁, 3-12 member heterocycle which is unsubstituted or substituted one or more times by R₁₂, or 4-18 member heterocycle-alkyl which is unsubstituted or substituted one or more times by R₁₂;
R₃ and R₃′ can also be taken together to form 5-12 member heteroaryl which is unsubstituted or substituted one or more times by R₁₁, or a 3-12 member heterocycle which is unsubstituted or substituted one or more times by R₁₂;
R₄ is C₁₋₁₂ alkyl which is unsubstituted or substituted one or more times by R₁₀, C₂₋₁₂ alkenyl which is unsubstituted or substituted one or more times by R₁₀, C₂₋₁₂ alkynyl which is unsubstituted or substituted one or more times by R₁₀, C₆₋₁₄ aryl which is unsubstituted or substituted one or more times by R₁₁, C₇₋₁₆ aralkyl which is unsubstituted or substituted one or more times by R₁₁, 5-12 member heteroaryl which is unsubstituted or substituted one or more times by R₁₁, 6-18 member heteroaralkyl which is unsubstituted or substituted one or more times by R₁₁, 3-12 member heterocycle which is unsubstituted or substituted one or more times by R₁₂, or 4-18 member heterocycle-alkyl which is unsubstituted or substituted one or more times by R¹²;
R₅ and R₆ are each independently C₁₋₁₂ alkyl which is unsubstituted or substituted one or more times by R₁₀, C₂₋₁₂ alkenyl which is unsubstituted or substituted one or more times by R₁₀, C₂₋₁₂ alkynyl which is unsubstituted or substituted one or more times by R₁₀, C₆₋₁₄ aryl which is unsubstituted or substituted one or more times by R₁₁, C₇₋₁₆ aralkyl which is unsubstituted or substituted one or more times by R₁₁, 5-12 member heteroaryl which is unsubstituted or substituted one or more times by R₁₁, 6-18 member heteroaralkyl which is unsubstituted or substituted one or more times by R₁₁, 3-12 member heterocycle which is unsubstituted or substituted one or more times by R₁₂, or 4-18 member heterocycle-alkyl which is unsubstituted or substituted one or more times by R₁₂;
R¹⁰ is halogen, oxo, alkoxy, —NH₂, —NH(C₁₋₄ alkyl), —N(C₁₋₄alkyl)₂, —C(O)NH₂, —C(O)NH(C₁₋₄ alkyl), —C(O)N(C₁₋₄ alkyl)₂, —NHC(O)H, alkyl)C(O)H, alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄ alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino;
R¹¹ is halogen, C₁₋₆ alkyl, halogenated C₁₋₆ alkyl, C₂₋₆ alkenyl, C₂₋₆ alkynyl, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄ alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino; and
R¹² is halogen, oxo, C₁₋₆ alkyl, halogenated C₁₋₆ alkyl, C₂₋₆ alkenyl, C₂₋₆ alkynyl, C₁₋₆ alkoxy, —NH₂, —NH(C₁₋₄ alkyl), —N(C₁₋₄ alkyl)₂, —C(O)NH₂, —C(O)NH(C₁₋₄ alkyl), —C(O)N(C₁₋₄ alkyl)₂, —NHC(O)H, —N(C₁₋₄ alkyl)C(O)H, —N(C₁₋₄ alkyl)C(O)C₁₋₄ alkyl, —NHC(O)C₁₋₄ alkyl, —NHC(O)OC₁₋₄ alkyl, —N(C₁₋₄ alkyl)C(O)OC₁₋₄ alkyl, —NHC(O)NH₂, —N(C₁₋₄ alkyl)C(O)NH₂, —NHC(O)NHC₁₋₄ alkyl, —N(C₁₋₄ alkyl)C(O)NHC₁₋₄ alkyl, —N(C₁₋₄ alkyl)C(O)N(C₁₋₄ alkyl)₂, —NHC(O)N(C₁₋₄ alkyl)₂, —C(O)H, —C(O)C₁₋₄ alkyl, C(O)OH, —C(O)OC₁₋₄ alkyl, —OC(O)C₁₋₄ alkyl, —OC(O)NH(C₁₋₄ alkyl), —OC(O)N(C₁₋₄ alkyl)₂, —C(NOH)C₁₋₄ alkyl, —C(NOH)H, —C(NOC₁₋₄ alkyl)C₁₋₄ alkyl, —C(NOC₁₋₄ alkyl)H, hydroxyl, nitro, azido, cyano, —S(O)₀₋₃H, —S(O)₀₋₃C₁₋₄ alkyl, —SO₂NH₂, —SO₂NH(C₁₋₄ alkyl), —SO₂N(C₁₋₄ alkyl)₂, —N(C₁₋₄ alkyl)SO₂C₁₋₄ alkyl, —NHSO₂C₁₋₄ alkyl, —P(O)(OH)₂, —P(O)(OC₁₋₄alkyl)OH, —P(O)(OC₁₋₄alkyl)₂, amidino, or guanidino;

2. (canceled)

3. A compound according to claim 1, wherein said compound is defined by formula (Ic):

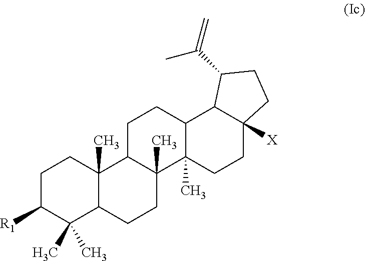

4. (canceled)

5. A compound according to claim 3, wherein R¹ is O-3′,3′-dimethylsuccinyl.

6-10. (canceled)

11. A compound according to claim 5, wherein R₂ is H.

12. (canceled)

13. A compound according to claim 1, wherein X is:

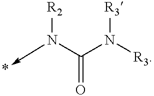

14. A compound according to claim 1, wherein X is:

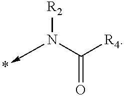

15-23. (canceled)

24. A compound according to claim 13, wherein R₃′ is H, methyl, ethyl, propyl, isopropyl, butyl, sec-butyl, tert-butyl, cyclopropyl, cyclobutyl, cyclopentyl, or cyclohexyl.

25. (canceled)

26. A compound according to claim 13, wherein R₃′ is H.

27-35. (canceled)

36. A compound according to claim 26, wherein R₃ is 5-6 member heteroaryl which is unsubstituted or substituted one or more times by R¹¹.

37. A compound according to claim 36, wherein R₃ is oxadiazole which is unsubstituted or substituted one or more times by R¹¹.

38. A compound according to claim 37, wherein R₃ is oxadiazole which is unsubstituted or substituted by one methyl.

39. A compound according to claim 24, wherein R₃ is methyl, ethyl, propyl, isopropyl, butyl, sec-butyl, or tert-butyl.

40. A compound according to claim 39, wherein R₃ is methyl.

41. A compound according to claim 26, wherein R₃ is benzyl which is unsubstituted or substituted one or more times by R¹¹.

42. A compound according to claim 41, wherein R₃ is benzyl.

43. (canceled)

44. A compound according to claim 14, wherein R⁴ is C₁₋₆ alkyl which is unsubstituted or substituted one or more times by R₁₀, phenyl which is unsubstituted or substituted one or more times by R₁₁, benzyl which is unsubstituted or substituted one or more times by R₁₁, 5-6 member heteroaryl which is unsubstituted or substituted one or more times by R₁₁, 7-8 member heteroaralkyl which is unsubstituted or substituted one or more times by R₁₁, 5-6 member heterocycle which is unsubstituted or substituted one or more times by R₁₂, or 7-8 member heterocycle-alkyl which is unsubstituted or substituted one or more times by R₁₂.

45. (canceled)

46. A compound according to claim 44, wherein R₄ is heterocycle-alkyl which is pyrrolidinyl ethyl.

47. (canceled)

48. (canceled)

49. (canceled)

50. (canceled)

51. A compound according to claim 44, wherein R₄ is benzyl which is unsubstituted or substituted one or more times by R₁₁.

52. A compound according to claim 44, wherein R₄ is benzyl.

53-64. (canceled)

65. A compound according to claim 1, wherein
R¹⁰ is halogen, hydroxyl, or C₁₋₃ alkoxy.
R¹¹ is halogen, C₁₋₃ alkyl, halogenated C₁₋₃ alkyl, hydroxyl, or C₁₋₃ alkoxy.
R¹² is halogen, oxo, C₁₋₃ alkyl, halogenated C₁₋₃ alkyl, hydroxyl, or C₁₋₃ alkoxy.

66. A compound according to claim 1 selected from:
3β-O-(3′,3′-Dimethylsuccinyl)-17β-tert-butyloxycarbonylamino-28-norlup-20(29)-ene;
3β-O-(2′,2′-Dimethylsuccinyl)-17β-tert-butyloxycarbonylamino-28-norlup-20(29)-ene;
3β-O-[(1′S,3′R)-2′,2′,3′-Trimethyl-cyclopentane-3′-carboxylic acid-1′-carboxyl]-17β-tert-butyloxycarbonylamino-28-norlup-20(29)-ene;
3β-O-(cis-Cyclohexane-3′-carboxylic acid-1′-carboxyl)-17β-[N tert-butyloxycarbonyl-amino]-28-norlup-20(29)-ene;
3β-O-(3′,3′-Dimethylsuccinyl)-17β-[N′-(methoxycarbonyl)ureido]-28-norlup-20(29)-ene;
3β-O-(3′,3′-Dimethylsuccinyl)-17β-[N′-(tert-butyl)ureido]-28-norlup-20(29)-ene;
3β-O-(3′,3′-Dimethylsuccinyl)-28-norlup-20(29)-ene-17β-yl-N-carbamoyl-L-valine methyl ester;
3β-O-(3′,3′-Dimethylsuccinyl)-28-norlup-20(29)-ene-17β-yl-N-carbamoyl-L-valine;
3β-O-(3′,3′-Dimethylglutaryl)-28-norlup-20(29)-ene-17β-yl-N-carbamoyl-L-valine;
3β-O-(3′,3′-Dimethylsuccinyl)-17β-[N′-(benzyl)ureido]-28-norlup-20(29)-ene;
3β-O-(3′,3′-Dimethylsuccinyl)-17β-[N′-(methyl)ureido]-28-norlup-20(29)-ene;
3β-O-(3′,3′-Dimethylsuccinyl)-17β-[(morpholine-4-carbonyl)-amino]-28-norlup-20(29)-ene;
3β-O-(3′,3′-Dimethylsuccinyl)-17β-[(4-methyl-piperazine-1-carbonyl)-amino]-28-norlup-20(29)-ene;
3β-O-(3′,3′-Dimethylsuccinyl)-17β-ureido-28-norlup-20(29)-ene;
3β-O-(3′,3′-Dimethylsuccinyl)-17β-[(piperidine-1-carbonyl)-amino]-28-norlup-20(29)-ene;
3β-O-(3′,3′-Dimethylsuccinyl)-17β-(N′-phenyl-ureido)-28-norlup-20(29)-ene;
3β-O-(3′,3′-Dimethylsuccinyl)-17β-(N′,N′-dimethyl-ureido)-28-norlup-20(29)-ene;
3β-O-(3′,3′-Dimethylsuccinyl)-17β-[N′-(1-methyl-piperidin-4-ylmethyl)-ureido]-28-norlup-20(29)-ene;
3β-O-(3′,3′-Dimethylsuccinyl)-17β-[3-(5-methyl-[1,3,4]oxadiazol-2-yl)-ureido]-28-norlup-20(29)-ene;
3β-O-(3′,3′-Dimethylsuccinyl)-17β-(N′-isopropyl-ureido)-28-norlup-20(29)-ene;
3β-O-(3′,3′-Dimethylsuccinyl)-17β-(N′-4-fluorophenyl-ureido)-28-norlup-20(29)-ene;
3β-O-(3′,3′-Dimethylsuccinyl)-17β-(N′-4-fluorophenylmethyl-ureido)-28-norlup-20(29)-ene;
3β-O-(3′,3′-Dimethylsuccinyl)-17β-(N′-thiazol-2-yl-ureido)-28-norlup-20(29)-ene;
3β-O-(3′,3′-Dimethylsuccinyl)-17β-(N′-cyclohexylmethyl-ureido)-28-norlup-20(29)-ene;
3β-O-(3′,3′-Dimethylsuccinyl)-17β-(N′-tetrahydropyran-4-ylmethyl-ureido)-28-norlup-20(29)-ene;
3β-O-(3′,3′-Dimethylsuccinyl)-17β-(N′-cyclohexyl-ureido)-28-norlup-20(29)-ene;
3β-O-(3′,3′-Dimethylsuccinyl)-17β-(N′-(S)-1-phenyl-ethyl-ureido)-28-norlup-20(29)-ene;
3β-O-(3′,3′-Dimethylsuccinyl)-17β-(N′-isobutylureido)-28-norlup-20(29)-ene;
3β-O-(3′,3′-Dimethylsuccinyl)-17β-(N′-4,4-difluorocyclohexyl-ureido)-28-norlup-20(29)-ene;
3β-O-(3′,3′-Dimethylsuccinyl)-17β-(N′-pyridin-4-yl-ureido)-28-norlup-20(29)-ene;
3β-O-(3′,3′-Dimethylsuccinyl)-17β-(N′-(R)-1-phenyl-ethyl-ureido)-28-norlup-20(29)-ene;
3β-O-(3′,3′-Dimethylsuccinyl)-17β-[N′-(1-methyl-1-phenylethyl)-ureido]-28-norlup-20(29)-ene;
3β-O-(3′,3′-Dimethylsuccinyl)-17β-[(pyrrolidine-1-carbonyl)-amino]-28-norlup-20(29)-ene;
3β-O-(3′,3′-Dimethylsuccinyl)-17β-methylsulfonylamino-28-norlup-20(29)-ene;
17β-Acetylamino-3β-O-(3′,3′-dimethylsuccinyl)-28-norlup-20(29)-ene;
3β-O-(3′,3′-Dimethylsuccinyl)-17β-methoxyoxalyl-amino-28-norlup-20(29)-ene;
17β-Dimethylaminooxalyl-amino-3β-O-(3′,3′-dimethylsuccinyl)-28-norlup-20(29)-ene;
3β-O,17β-N-bis(3′,3′-dimethylsuccinyl)-28-norlup-20(29)-ene;
3β-O,17β-N-bis(3′,3′-dimethylglutaryl)-28-norlup-20(29)-ene;
3β-O-(3′,3′-Dimethylsuccinyl)-17β-phenylacetylamino-28-norlup-20(29)-ene;
17β-Benzoylamino-3β-O-(3′,3′-dimethylsuccinyl)-28-norlup-20(29)-ene;
3β-O-(3′,3′-Dimethylsuccinyl)-17β-(pyridin-4-ylcarbonyl)-amino-28-norlup-20(29)-ene;
17β-(Cyclopropanecarbonyl-amino)-3β-O-(3′,3′-dimethylsuccinyl)-28-norlup-20(29)-ene;
3β-O-(3′,3′-Dimethylsuccinyl)-17β-isobutyrylamino-28-norlup-20(29)-ene;
3β-O-(3′,3′-Dimethylsuccinyl)-17β-(3-pyrrolidin-1-yl-propionylamino)-28-norlup-20(29)-ene;
3β-O-(3′,3′-Dimethylsuccinyl)-17β-[(5-methyl-[1,3,4]oxadiazole-2-carbonyl)-amino]-28-norlup-20(29)-ene;
3β-O-(3′,3′-Dimethylsuccinyl)-17β-[(thiazol-4-ylcarbonyl)-amino]-28-norlup-20(29)-ene;
3β-O-(3′,3′-Dimethylsuccinyl)-17β-[(1-methyl-1H-pyrazol-4-ylcarbonyl)-amino]-28-norlup-20(29)-ene;
3β-O-(cis-Cyclohexane-3′-carboxylic acid-1′-carboxyl)-17β-(pyridin-4-ylcarbonyl)-amino-28-norlup-20(29)-ene;
3β-O-(3′,3′-Dimethylsuccinyl)-17β-methoxycarbonylamino-28-norlup-20(29)-ene;
3β-O-(3′,3′-Dimethylsuccinyl)-17β-benzyloxycarbonylamino-28-norlup-20(29)-ene;
17β-Amino-3β-O-(3′,3′-dimethylsuccinyl)-28-norlupane;
3β-O-(3′,3′-Dimethylsuccinyl)-17β-acetylamino-28-norlupane;
3β-O-(3′,3′-Dimethylsuccinyl)-17β-methoxycabonylamino-28-norlupane;
and pharmaceutically acceptable salts thereof.

67. A compound according to claim 1 selected from
3β-O-(3′,3′-Dimethylsuccinyl)-17β-tert-butyloxycarbonylamino-28-norlup-20(29)-ene;
3β-O-(3′,3′-Dimethylsuccinyl)-17β-[N′-(benzyl)ureido]-28-norlup-20(29)-ene;
3β-O-(3′,3′-Dimethylsuccinyl)-17β-[(morpholine-4-carbonyl)-amino]-28-norlup-20(29)-ene;
3β-O-(3′,3′-Dimethylsuccinyl)-17β-[(4-methyl-piperazine-1-carbonyl)-amino]-28-norlup-20(29)-ene;
3β-O-(3′,3′-Dimethylsuccinyl)-17β-[(piperidine-1-carbonyl)-amino]-28-norlup-20(29)-ene;
3β-O-(3′,3′-Dimethylsuccinyl)-17β-(N′-phenyl-ureido)-28-norlup-20(29)-ene;
3β-O-(3′,3′-Dimethylsuccinyl)-17β-(N′,N′-dimethyl-ureido)-28-norlup-20(29)-ene;
3β-O-(3′,3′-Dimethylsuccinyl)-17β-[N′-(1-methyl-piperidin-4-ylmethyl)-ureido]-28-norlup-20(29)-ene;
3β-O-(3′,3′-Dimethylsuccinyl)-17β-[3-(5-methyl-[1,3,4]oxadiazol-2-yl)-ureido]-28-norlup-20(29)-ene;
3β-O-(3′,3′-Dimethylsuccinyl)-17β-phenylacetylamino-28-norlup-20(29)-ene;
17β-Benzoylamino-3β-O-(3′,3′-dimethylsuccinyl)-28-norlup-20(29)-ene;
3β-O-(3′,3′-Dimethylsuccinyl)-17β-(pyridin-4-ylcarbonyl)-amino-28-norlup-20(29)-ene;
3β-O-(3′,3′-Dimethylsuccinyl)-17β-(3-pyrrolidin-1-yl-propionylamino)-28-norlup-20(29)-ene;
3β-O-(3′,3′-Dimethylsuccinyl)-17β-[(5-methyl-[1,3,4]oxadiazole-2-carbonyl)-amino]-28-norlup-20(29)-ene;
3β-O-(3′,3′-Dimethylsuccinyl)-17β-methoxycarbonylamino-28-norlup-20(29)-ene;
3β-O-(3′,3′-Dimethylsuccinyl)-17β-benzyloxycarbonylamino-28-norlup-20(29)-ene;
and pharmaceutically acceptable salts thereof.

68. A compound according to claim 1 selected from
17β-tert-Butyloxycarbonylamino-3β-hydroxy-28-norlup-20(29)-ene;
17β-Amino-3β-hydroxy-28-norlup-20(29)-ene;
17β-Amino-3β-O-(3′,3′-dimethylsuccinyl)-28-norlup-20(29)-ene;
3β-O-[(1′S,3′R)-2′,2′,3′-Trimethyl-cyclopentane-3′-carboxylic acid-1′-carboxyl]-17β-amino-28-norlup-20(29)-ene;
3β-O-(cis-Cyclohexane-3′-carboxylic acid-1′-carboxyl)-17β-amino-28-norlup-20(29)-ene;
17β-Methyl-amino-3β-O-(3′,3′-dimethylsuccinyl)-28-norlup-20(29)-ene;
17β-(Cyclopropylmethyl-amino)-3β-O-(3′,3′-dimethylsuccinyl)-28-norlup-20(29)-ene;
3β-O-(3′,3′-Dimethylsuccinyl)-17β-dimethylamino-28-norlup-20(29)-ene;
and pharmaceutically acceptable salts thereof.

69. A compound according to claim 1 selected from
17β-benzoylamino-3β-hydroxy-28-norlup-20(29)-ene;
3β-hydroxy-17β-(pyridin-4-ylcarbonyl)-amino-28-norlup-20(29)-ene;
3β-hydroxy-17β-[N′-(tert-butyl)ureido]-28-norlup-20(29)-ene;
3β-hydroxy-28-norlup-20(29)-ene-17β-yl-N-carbamoyl-L-valine;
3β-hydroxy-17β-[N′-(benzyl)ureido]-28-norlup-20(29)-ene;
3β-hydroxy-17β-[N′-(methyl)ureido]-28-norlup-20(29)-ene;
3β-hydroxy-17β-methoxycarbonylamino-28-norlup-20(29)-ene;
3β-hydroxy-17β-benzyloxycarbonylamino-28-norlup-20(29)-ene;
and pharmaceutically acceptable salts thereof.

70-88. (canceled)

89. A pharmaceutical composition comprising a compound according to claim 1 together with one or more pharmaceutically acceptable carriers or excipients.

90. A method for prevention or treatment of HIV infection in a subject in need of such treatment comprising administering to the subject a therapeutically effective amount of a compound according to claim 1.

91. (canceled)
